(19) United States
(12) Patent Application Publication
(10) Pub. No.: US 2023/0106107 A1
(43) Pub. Date: Apr. 6, 2023

(54) METHODS AND SYSTEMS FOR DETERMINING PIGMENTATION PHENOTYPES

(71) Applicant: Embark Veterinary, Inc., Boston, MA (US)

(72) Inventors: Adam Boyko, Ithaca, NY (US); Aaron Sams, Ithaca, NY (US); Andrea Slavney, Ithaca, NY (US); Meghan Jensen, Maine, NY (US); Petra Deane, Weybridge, VT (US); Takeshi Kawakami, Ithaca, NY (US)

(21) Appl. No.: 17/956,446

(22) Filed: Sep. 29, 2022

Related U.S. Application Data
(63) Continuation of application No. PCT/US2021/025433, filed on Apr. 1, 2021.
(60) Provisional application No. 63/004,204, filed on Apr. 2, 2020.

(51) Int. Cl. G16B 20/20; G16B 40/20

(52) U.S. Cl. CPC G16B 20/20; G16B 40/20

(57) ABSTRACT

The present disclosure provides methods, systems, and media for determining a pigmentation phenotype of a canine subject. In an aspect, the present disclosure provides a computer-implemented method for determining a pigmentation phenotype of a canine subject. The method may comprise (a) receiving genotype data for the canine subject, wherein the genotype data comprises quantitative values of each of a plurality of genetic markers, wherein the plurality of genetic markers comprises genetic variants; (b) applying a trained machine learning classifier to the genotype data to determine a predicted pigmentation phenotype based at least in part on the quantitative values of the plurality of genetic variants; and (c) identifying the canine subject as having the predicted pigmentation phenotype with an accuracy of at least about 70%.

### CROSS-REFERENCE

This application is a continuation of International Application No. PCT/US2021/025433, filed Apr. 1, 2021, which claims the benefit of U.S. Provisional Application No. 63/004,204, filed Apr. 2, 2020, all of which are incorporated by reference herein in their entirety.

### BACKGROUND

Consumer genomics may enable genetic discovery on an unprecedented scale by linking very large databases of personal genomic data with phenotype information voluntarily submitted via web-based surveys. These databases may have a transformative effect on human genomics research, yielding insights on increasingly complex traits, behaviors, and disease by including many thousands of individuals in genome-wide association studies (GWAS). The promise of consumer genomic data may not be limited to human research, however. Genomic tools for canine subjects (e.g., dogs) may be readily available, with hundreds of causal Mendelian variants already characterized, because selection and breeding may lead to dramatic phenotypic diversity underlain by a simple genetic structure.

### SUMMARY

The present disclosure provides methods, systems, and media for determining a pigmentation phenotype of a canine subject. In an aspect, the present disclosure provides a computer-implemented method for determining a pigmentation phenotype of a canine subject, comprising (a) receiving genotype data for the canine subject, wherein the genotype data comprises quantitative values of each of a plurality of genetic markers, wherein the plurality of genetic markers comprises genetic variants; (b) applying a trained machine learning classifier to the genotype data to determine a predicted pigmentation phenotype based at least in part on the quantitative values of the plurality of genetic variants; and (c) identifying the canine subject as having the predicted pigmentation phenotype with an accuracy of at least about 70%.

In some embodiments, the canine subject is a dog. In some embodiments, the dog is a purebred dog or a mixed breed dog. In some embodiments, the dog is a purebred dog. In some embodiments, the purebred dog is selected from Labrador retriever and golden retriever. In some embodiments, the dog is a mixed breed dog. In some embodiments, the dog has a breed selected from Labrador retriever and golden retriever.

In some embodiments, the genotype data is obtained by assaying a biological sample obtained from the canine subject. In some embodiments, the biological sample comprises a blood sample, a saliva sample, a swab sample, a cell sample, or a tissue sample. In some embodiments, the assaying comprises sequencing the biological sample or derivatives thereof. In some embodiments, the plurality of genetic markers comprises at least 5 distinct genetic markers. In some embodiments, the plurality of genetic markers comprises at least 10 distinct genetic markers. In some embodiments, the quantitative values are indicative of a presence or absence in the genotype data of each of the plurality of genetic variants. In some embodiments, the plurality of genetic variants is selected from the group consisting of single nucleotide polymorphisms (SNPs), insertions or deletions (indels), microsatellites, or structural variants.

In some embodiments, the pigmentation phenotype comprises a coat color intensity phenotype, a ticking phenotype, a roaning phenotype, or a tongue pigmentation phenotype. In some embodiments, the pigmentation phenotype comprises a coat color intensity phenotype. In some embodiments, the plurality of genetic markers comprises one or more markers selected from the group listed in Table 8. In some embodiments, the plurality of genetic markers comprises one or more SNPs of a genetic locus selected from canFam3.1 chr2: 74.7 Mb, chr20: 55.8 Mb, and chr21: 10.9 Mb. In some embodiments, the plurality of genetic markers comprises canFam3.1 chr2: 74.7 Mb or chr21: 10.9 Mb. In some embodiments, the plurality of genetic markers comprises canFam3.1 chr2: 74.7 Mb and chr21: 10.9 Mb. In some embodiments, the pigmentation phenotype comprises a ticking phenotype. In some embodiments, the pigmentation phenotype comprises a roaning phenotype. In some embodiments, the plurality of genetic markers comprises one or more markers selected from the group listed in Table 11. In some embodiments, the pigmentation phenotype comprises a tongue pigmentation phenotype. In some embodiments, the plurality of genetic markers comprises one or more markers selected from the group listed in Table 13.

In some embodiments, applying the trained machine learning classifier comprises determining a weighted sum of the quantitative values of the plurality of genetic markers. In some embodiments, the weighted sum is determined using a plurality of pre-determined weights associated with the plurality of genetic markers. In some embodiments, the plurality of pre-determined weights associated with the plurality of genetic markers is determined by performing a genome-wide association study (GWAS) comprising a multiple linear regression. In some embodiments, applying the trained machine learning classifier comprises applying a multiple logistic regression to the quantitative values of the plurality of genetic markers.

In some embodiments, the method further comprises determining a second pigmentation phenotype of a second canine subject, and determining an expected range of pigmentation phenotypes of a potential offspring of the canine subject and the second canine subject. In some embodiments, the method further comprises determining a recommendation indicative of whether or not to breed the first canine subject and the second canine subject together, based on the expected range of pigmentation phenotypes of the potential offspring of the canine subject and the second canine subject. In some embodiments, the method further comprises determining a recommendation indicative of breeding the first canine subject and the second canine subject together, when the expected range of pigmentation phenotypes of the potential offspring of the canine subject and the second canine subject includes a pre-determined pigmentation phenotype. In some embodiments, the method further comprises determining a recommendation against breeding the first canine subject and the second canine subject together, when the expected range of pigmentation phenotypes of the potential offspring of the canine subject and the second canine subject does not include a pre-determined pigmentation phenotype. In some embodiments, the method further comprises generating a social connection between a first person associated with the first canine subject and a second person associated with the second canine subject, based at least in part on the expected range of pigmentation phenotypes of the potential offspring of the canine subject and the second canine subject. In some embodiments, the social connection is generated when the expected range of pigmentation phenotypes of the potential offspring of the canine subject and the second canine subject includes a pre-determined pigmentation phenotype. In some embodiments, the social connection is generated through a social media network. In some embodiments, the first person is a pet owner of the first canine subject, and wherein the second person is a pet owner of the second canine subject.

In some embodiments, the method further comprises identifying the canine subject as having the predicted pigmentation phenotype among at least 3 different categorical or quantitative values of pigmentation phenotypes. In some embodiments, the method further comprises identifying the canine subject as having the predicted pigmentation phenotype among at least 6 different categorical or quantitative values of pigmentation phenotypes. In some embodiments, the method further comprises identifying the canine subject as having the predicted pigmentation phenotype with an accuracy of at least about 75%. In some embodiments, the method further comprises identifying the canine subject as having the predicted pigmentation phenotype with an accuracy of at least about 80%. In some embodiments, the method further comprises identifying the canine subject as having the predicted pigmentation phenotype among 2 different categorical or quantitative values of pigmentation phenotypes. In some embodiments, the 2 different categorical or quantitative values of pigmentation phenotypes comprise a darker coat color and a lighter coat color. In some embodiments, the method further comprises identifying the canine subject as having the predicted pigmentation phenotype with an accuracy of at least about 85%. In some embodiments, the method further comprises identifying the canine subject as having the predicted pigmentation phenotype with an accuracy of at least about 90%. In some embodiments, the method further comprises identifying the canine subject as having the predicted pigmentation phenotype with an accuracy of at least about 95%.

In some embodiments, the trained machine learning classifier comprises a linear regression or a logistic regression. In some embodiments, the trained machine learning classifier comprises the linear regression. In some embodiments, the trained machine learning classifier comprises the logistic regression.

In another aspect, the present disclosure provides a computer system for determining a pigmentation phenotype of a canine subject, comprising: a database that is configured to store genotype data for the canine subject, wherein the genotype data comprises quantitative values of each of a plurality of genetic markers, wherein the plurality of genetic markers comprises genetic variants; and one or more computer processors operatively coupled to the database, wherein the one or more computer processors are individually or collectively programmed to: (a) apply a trained machine learning classifier to the genotype data to determine a predicted pigmentation phenotype based at least in part on the quantitative values of the plurality of genetic variants; and (b) identify the canine subject as having the predicted pigmentation phenotype with an accuracy of at least about 70%.

In another aspect, the present disclosure provides a non-transitory computer-readable medium comprising machine-executable code that, upon execution by one or more computer processors, implements a method for determining a pigmentation phenotype of a canine subject, the method comprising (a) receiving genotype data for the canine subject, wherein the genotype data comprises quantitative values of each of a plurality of genetic markers, wherein the plurality of genetic markers comprises genetic variants; (b) applying a trained machine learning classifier to the genotype data to determine a predicted pigmentation phenotype based at least in part on the quantitative values of the plurality of genetic variants; and (c) identifying the canine subject as having the predicted pigmentation phenotype with an accuracy of at least about 70%.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example of a method of determining a pigmentation phenotype of a canine subject.

FIG. 2 illustrates a computer system that is programmed or otherwise configured to implement methods provided herein.

FIGS. 3A-3B show Manhattan plots of association with roaning (FIG. 3A) and ticking (FIG. 3B). Red and blue horizontal lines are significant (P<5×10⁻⁸) and suggestive (P<1×10⁻⁵) associations, respectively.

FIGS. 4A-4B show a Q-Q plot of the association with roaning (FIG. 4A) and ticking (FIG. 4B). The GWAS of 320 roaned dogs (cases) and 357 non-ticked, non-roaned dogs (controls) identified two highly significant and two suggestive markers (FIG. 3A and FIGS. 4A-4B).

FIGS. 5A-5B show Manhattan plots of association with roaning, including roaning for herding breeds (FIG. 5A) and roaning for non-herding breeds (FIG. 5B). Red and blue horizontal lines are significant (P<5×10⁻⁸) and suggestive (P<1×10⁻⁵) associations, respectively.

FIGS. 6A-6B show Manhattan plots of association with ticking, including ticking for herding breeds (FIG. 6A) and ticking for non-herding breeds (FIG. 6B). Red and blue horizontal lines are significant (P<5×10⁻⁸) and suggestive (P<1×10⁻⁵) associations, respectively.

FIG. 7 shows normalized read depth in 5-kb sliding windows across the significant GWAS locus on CFA38 for Australian Cattle Dogs (red), German Wirehaired Pointer (pink), and Border Collies (grey). Filled circles shows the corresponding region of the Manhattan plot shown in FIGS. 3A-3B.

FIG. 8 shows haplotype structure near the tandem duplication on CFA38 (position 11,031,835-11,243,237). Border Collies (grey), breeds with high frequency of ticking (Brittany, Clumber spaniel, and English setter; purple), breeds with high frequency of roaning (Australian Cattle Dog, German Wirehaired Pointer, Wirehaired Pointer, and Wirehaired Pointing Griffon; brown), and Dalmatians (red). Rows correspond to haplotypes (two rows/individual), and columns correspond to markers. +/−: presence and absence of the 11-kb duplication based on Manta. Red box: 11-kb duplication (CFA38:11,131,835-11,143,234). Orange box: a core haplotype (CFA38:11,122,646-11,167,876).

FIG. 9 shows discordant read pairs at the duplication breakpoint on CFA38 identified in Munsterlander (top panel), Australian Cattle Dog (middle: SRR7107580), and Border Collie (bottom: SRR7107950). Outward-facing read pairs (green) indicate that this is a tandem duplication found in ticked and roaned dogs but not in Border Collie.

FIGS. 10A-10B show PCR genotyping of the tandem duplication on CFA38 associated with roaning. FIG. 10A shows a schematic view of the design of the PCR genotyping assay. Single headed arrows indicate three pairs of primers to amplify three regions. The first (black) and the third (yellow) primer pairs should produce amplicons in all dogs regardless of the presence or absence of the duplication, while the second pair in the middle should produce an amplicon only in dogs carrying the duplication. FIG. 10B shows PCR genotyping of a roaned and control dogs. Each gel lane corresponds to PCR primer pairs depicted in FIG. 10A.

FIG. 11 shows a density distribution of ΔLRR for the discovery panel dogs with zero, one, or two copies of the duplication-associated haplotypes (no haplotype, heterozygote, and homozygote, respectively). Vertical ticks indicate individual ΔLRR of dogs with roaning (orange) and without roaning (grey). Density plots with the number of individuals less than 10 are not shown, but individual ΔLRR is indicated with longer vertical ticks.

FIG. 12 shows genotype frequency of the marker near MITF (CFA20:21836232) in roaned and non-roaned dogs. Using GWAS, a marker near MITF (CFA20:21,836,232) was identified as a region potentially associated with roaning. Roaned dogs were mostly “GG” homozygous (89%) or “AG” heterozygous (10%) at this marker, while “AA” homozygotes were most common in non-roaned dogs (66%), affirming the requirement of a capability of having white areas for roaning to be visible.

FIGS. 13A-13B show a density distribution of ΔLRR for the validation panel dogs with zero, one, or two copies of the duplication-associated haplotypes (no haplotype, heterozygote, and homozygote, respectively), including target breeds (FIG. 13A) and mixed breeds (FIG. 13B). Vertical ticks indicate individual ΔLRR of dogs with roaning (orange) and without roaning (grey). Density plots with the number of individuals less than 10 are not shown, but individual ΔLRR is indicated with longer vertical ticks.

FIGS. 14A-14D show a signature of selection in the region on CFA37 associated with roaning. FIG. 14A shows nucleotide diversity (π) for Wirehaired Pointing Griffon (orange), Border Collies (grey squares), and Labrador Retriever (black triangles) in 500-kb sliding windows. FIG. 14B shows pairwise genetic differentiation (F_{ST}) for Wirehaired Pointing Griffon (red) and Labrador Retriever (black). Border Collies were used as a reference. FIG. 14C shows ROH in Australian Cattle Dog (orange), Dalmatians (red), and Border Collies (grey).

FIG. 14D shows XP-EHH in Australian Cattle Dog (orange), Dalmatians (red), and Labrador Retrievers (black). Border Collies were used as a reference. Wirehaired Pointing Griffons and Australian Cattle Dogs are commonly associated with roaning. Blue rectangle: position of the 11-kb duplication. π and F_{ST} are estimated by using whole-genome resequencing data, while ROH and XP-EHH were estimated by using Illumina genotyping data.

FIG. 15 shows human orthologous region (hg38) of the CFA38 associated with roaning (UCSC genome browser). The highlighted area in blue is the orthologous region to the tandem duplication identified in dogs with roaning, which is located within the intron 61 of USH2A. GeneHancer Regulatory Elements are located at chr1:215,715,579-215,717,032 (green line), which corresponds to CFA38:11,146,170-11,147,605 in the dog genome. DNAse I hypersensitive sites: grey and black boxes. Open Regulatory Annotation (ORegAnno): orange and blue boxes.

FIGS. 16A-16H show representative coat phenotypes, including German Wirehaired Pointer (roaned) (FIG. 16A); Australian Cattle Dog (roaned) (FIG. 16B); a mixed breed of Treeing Walker Coonhound and Bluetick Coonhound (ticked) (FIG. 16C); a Border Collie (ticked) (FIG. 16D); an English Setter (both roaned and ticked) (FIG. 16E); an Australian Cattle Dog (both roaned and ticked) (FIG. 16F); a Pointer (without roaning and ticking) (FIG. 16G); and an Australian Cattle Dog (without roaning and ticking) (FIG. 16H). FIGS. 16A, 16C, 16E, and 16G are non-herding breeds, while FIGS. 16B, 16D, 16F, and 16H are herding breeds.

FIGS. 17A-17B show Manhattan plots of association with roaning and ticking, including for Roaning (FIG. 17A) and Ticking (FIG. 17B).

FIG. 18 shows normalized read depth in 5-kb sliding windows across the significant GWAS locus on CFA38 for Australian Cattle Dogs, German Wirehaired Pointer, and Border Collies.

FIG. 19 shows haplotypes near the marker on CFA38 significantly associated with roaning. Border Collies, breeds with high frequency of ticking, breeds with high frequency of roaning, and Dalmatians.

FIGS. 20A-20B show PCR genotyping of the tandem duplication on CFA38 associated with roaning.

FIG. 21 shows density distribution of the array signal intensity (ΔLRR) for the discovery panel dogs with zero, one, or two copies of the duplication-associated haplotypes (no haplotype, heterozygote, and homozygote, respectively). Vertical ticks indicate individual ΔLRR of dogs with roaning (heterozygote and homozygote) and without roaning (no haplotype).

FIGS. 22A-22D show a signature of selection in the region on CFA38 associated with roaning.

FIGS. 23A-23C show the six point coat pheomelanin intensity scale.

FIGS. 24A-24B show quantitative coat pheomelanin intensity GWAS results.

FIGS. 25A-25B show species and breed allele frequencies at top GWAS markers.

FIGS. 26A-26B show dominance and epistatic interactions.

FIGS. 27A-27B show performance of the best fit multivariate linear regression classifier model for pheomelanin intensity phenotypes in validation cohort.

FIG. 28 shows phenotyping validation on 350 randomly selected dogs.

FIGS. 29A-29C show Manhattan plots for additional GWAS, including 6-point phenotype, no covariates (FIG. 29A); binary phenotype, with covariates (FIG. 29B); and binary phenotype, no covariates (FIG. 29C).

FIGS. 30A-30E show detailed views of regions surrounding top GWAS SNPs (e.g., on CFA2, CFA15, CFA18, CFA20, and CFA21), including CFA2 Association Region (74,465,672-75,100,435) (FIG. 30A); CFA15 Association Region (29,575,066-29,973,539) (FIG. 30B); CFA18 Association Region (12,410,382-13,410,382) (FIG. 30C); CFA20 Association Region (55,783,410-55,960,115) (FIG. 30D); and CFA21 Association Region (10,698,290-11,165,504) (FIG. 30E).

FIG. 31A shows that CFA15 top marker genotype correlates with sequencing coverage in known CNV.

FIG. 31B shows SRA run ID and sample name, breed, BICF2G630433130 genotype (coded as number of red-associated alleles), and CFA15 CNV mean normalized depth of coverage for all dogs shown in FIG. 31A.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a sample” includes a plurality of samples, including mixtures thereof.

As used herein, the term “subject,” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets (e.g., canines such as dogs, or felines such as cats). The subject may have a normal or abnormal health or physiological state or condition or be suspected of having a normal or abnormal health or physiological state or condition. The subject may be displaying a symptom(s) indicative of a health or physiological state or condition. As an alternative, the subject can be asymptomatic with respect to such health or physiological state or condition.

The term “nucleic acid,” or “polynucleotide,” as used herein, generally refers to a molecule comprising one or more nucleic acid subunits, or nucleotides. A nucleic acid may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (P03) groups. A nucleotide can include a nucleobase, a five-carbon sugar (either ribose or deoxyribose), and one or more phosphate groups, individually or in combination. Ribonucleotides are nucleotides in which the sugar is ribose. Deoxyribonucleotides are nucleotides in which the sugar is deoxyribose. A nucleotide can be a nucleoside monophosphate or a nucleoside polyphosphate. A nucleotide can be a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a nucleic acid is deoxyribonucleic acid (DNA), ribonucleic acid (RNA), or derivatives or variants thereof. A nucleic acid may be single-stranded or double stranded. A nucleic acid molecule may be linear, curved, or circular or any combination thereof.

The terms “nucleic acid molecule,” “nucleic acid sequence,” “nucleic acid fragment,” “oligonucleotide” and “polynucleotide,” as used herein, generally refer to a polynucleotide that may have various lengths, such as either deoxyribonucleotides or ribonucleotides (RNA), or analogs thereof. A nucleic acid molecule can have a length of at least about 5 bases, 10 bases, 20 bases, 30 bases, 40 bases, 50 bases, 60 bases, 70 bases, 80 bases, 90, 100 bases, 110 bases, 120 bases, 130 bases, 140 bases, 150 bases, 160 bases, 170 bases, 180 bases, 190 bases, 200 bases, 300 bases, 400 bases, 500 bases, 1 kilobase (kb), 2 kb, 3, kb, 4 kb, 5 kb, 10 kb, or 50 kb or it may have any number of bases between any two of the aforementioned values. An oligonucleotide is typically composed of a specific sequence of nucleotide bases: adenine (A); cytosine (C); guanine (G); and thymine (T) (uracil (U) for thymine (T) when the polynucleotide is RNA). Thus, the terms “nucleic acid molecule,” “nucleic acid sequence,” “nucleic acid fragment,” “oligonucleotide” and “polynucleotide” are at least in part intended to be the alphabetical representation of a polynucleotide molecule. Alternatively, the terms may be applied to the polynucleotide molecule itself. This alphabetical representation can be input into databases in a computer having a central processing unit and/or used for bioinformatics applications such as functional genomics and homology searching. Oligonucleotides may include one or more nonstandard nucleotide(s), nucleotide analog(s) and/or modified nucleotides.

The term “sample,” as used herein, generally refers to a biological sample. Examples of biological samples include nucleic acid molecules, amino acids, polypeptides, proteins, carbohydrates, fats, or viruses. In an example, a biological sample is a nucleic acid sample including one or more nucleic acid molecules. The biological sample may comprise or be derived from blood samples, saliva samples, swab samples, cell samples, or tissue samples. The nucleic acid molecules may be cell-free nucleic acid molecules, such as cell-free DNA (cfDNA) or cell-free RNA (cfRNA). The nucleic acid molecules may be derived from a variety of sources including human, mammal (e.g., dog), non-human mammal, ape, monkey, chimpanzee, reptilian, amphibian, or avian, sources. Further, samples may be extracted from a variety of animal fluids, including but not limited to bodily fluid samples such as blood, serum, plasma, vitreous, sputum, urine, tears, perspiration, saliva, semen, mucosal excretions, mucus, spinal fluid, cerebrospinal fluid (CSF), pleural fluid, peritoneal fluid, amniotic fluid, lymph fluid, and the like. Biological samples may be obtained or derived from subjects using an ethylenediaminetetraacetic acid (EDTA) collection tube, a cell-free RNA collection tube (e.g., Streck), or a cell-free DNA collection tube (e.g., Streck). Biological samples may be derived from whole blood samples by fractionation. Biological samples or derivatives thereof may contain cells. For example, a biological sample may be a blood sample or a derivative thereof (e.g., blood collected by a collection tube or blood drops) or a cell or tissue sample (e.g., a swab).

The term “whole blood,” as used herein, generally refers to a blood sample that has not been separated into sub-components (e.g., by centrifugation). The whole blood of a blood sample may contain cfDNA and/or germline DNA. Whole blood DNA (which may contain cfDNA and/or germline DNA) may be extracted from a blood sample. Whole blood DNA sequencing reads (which may contain cfDNA sequencing reads and/or germline DNA sequencing reads) may be extracted from whole blood DNA.

In an aspect, the present disclosure provides a computer-implemented method for determining a pigmentation phenotype of a canine subject, comprising (a) receiving genotype data for the canine subject, wherein the genotype data comprises quantitative values of each of a plurality of genetic markers, wherein the plurality of genetic markers comprises genetic variants; (b) applying a trained machine learning classifier to the genotype data to determine a predicted pigmentation phenotype based at least in part on the quantitative values of the plurality of genetic variants; and (c) identifying the canine subject as having the predicted pigmentation phenotype with an accuracy of at least about 70%.

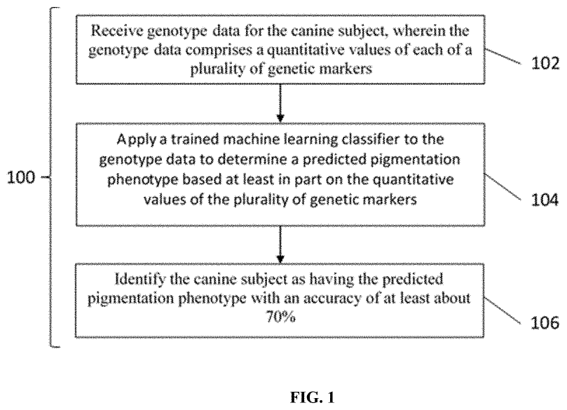

FIG. 1 illustrates an example of a method 100 for determining a pigmentation phenotype of a canine subject, in accordance with some embodiments. In operation 102, the method 100 may comprise receiving genotype data for the canine subject. For example, the genotype data may comprise quantitative values of each of a plurality of genetic markers. In some embodiments, the plurality of genetic markers comprises genetic variants. Next, in operation 104, the method 100 may comprise applying a trained machine learning classifier to the genotype data to determine a predicted pigmentation phenotype based at least in part on the quantitative values of the plurality of genetic markers (e.g., genetic variants). Next, in operation 106, the method 100 may comprise identifying the canine subject as having the predicted pigmentation phenotype with an accuracy of at least about 70%.

In some embodiments, the canine subject is a dog. In some embodiments, the dog is a purebred dog or a mixed breed dog. In some embodiments, the dog is a purebred dog. In some embodiments, the purebred dog is selected from Labrador retriever and golden retriever. In some embodiments, the dog is a mixed breed dog. In some embodiments, the dog has a breed selected from Labrador retriever and golden retriever.

In some embodiments, the genotype data is obtained by assaying a biological sample obtained from the canine subject. In some embodiments, the biological sample comprises a blood sample, a saliva sample, a swab sample, a cell sample, or a tissue sample. In some embodiments, the assaying comprises sequencing the biological sample or derivatives thereof. In some embodiments, the plurality of genetic markers comprises at least 5 distinct genetic markers. In some embodiments, the plurality of genetic markers comprises at least 10 distinct genetic markers. In some embodiments, the quantitative values are indicative of a presence or absence in the genotype data of each of the plurality of genetic variants. In some embodiments, the plurality of genetic variants is selected from the group consisting of single nucleotide polymorphisms (SNPs), insertions or deletions (indels), microsatellites, or structural variants.

In some embodiments, the pigmentation phenotype comprises a coat color intensity phenotype, a ticking phenotype, a roaning phenotype, or a tongue pigmentation phenotype. In some embodiments, the pigmentation phenotype comprises a coat color intensity phenotype. In some embodiments, the plurality of genetic markers comprises one or more markers selected from the group listed in Table 8. In some embodiments, the plurality of genetic markers comprises one or more SNPs of a genetic locus selected from canFam3.1 chr2: 74.7 Mb, chr20: 55.8 Mb, and chr21: 10.9 Mb. In some embodiments, the plurality of genetic markers comprises canFam3.1 chr2: 74.7 Mb or chr21: 10.9 Mb. In some embodiments, the plurality of genetic markers comprises canFam3.1 chr2: 74.7 Mb and chr21: 10.9 Mb. In some embodiments, the pigmentation phenotype comprises a ticking phenotype. In some embodiments, the pigmentation phenotype comprises a roaning phenotype. In some embodiments, the plurality of genetic markers comprises one or more markers selected from the group listed in Table 11. In some embodiments, the pigmentation phenotype comprises a tongue pigmentation phenotype. In some embodiments, the plurality of genetic markers comprises one or more markers selected from the group listed in Table 13.

In some embodiments, applying the trained machine learning classifier comprises determining a weighted sum of the quantitative values of the plurality of genetic markers. In some embodiments, the weighted sum is determined using a plurality of pre-determined weights associated with the plurality of genetic markers. In some embodiments, the plurality of pre-determined weights associated with the plurality of genetic markers is determined by performing a genome-wide association study (GWAS) comprising a multiple linear regression. In some embodiments, applying the trained machine learning classifier comprises applying a multiple logistic regression to the quantitative values of the plurality of genetic markers.

In some embodiments, the method further comprises determining a second pigmentation phenotype of a second canine subject, and determining an expected range of pigmentation phenotypes of a potential offspring of the canine subject and the second canine subject. In some embodiments, the method further comprises determining a recommendation indicative of whether or not to breed the first canine subject and the second canine subject together, based on the expected range of pigmentation phenotypes of the potential offspring of the canine subject and the second canine subject. In some embodiments, the method further comprises determining a recommendation indicative of breeding the first canine subject and the second canine subject together, when the expected range of pigmentation phenotypes of the potential offspring of the canine subject and the second canine subject includes a pre-determined pigmentation phenotype. In some embodiments, the method further comprises determining a recommendation against breeding the first canine subject and the second canine subject together, when the expected range of pigmentation phenotypes of the potential offspring of the canine subject and the second canine subject does not include a pre-determined pigmentation phenotype. In some embodiments, the method further comprises generating a social connection between a first person associated with the first canine subject and a second person associated with the second canine subject, based at least in part on the expected range of pigmentation phenotypes of the potential offspring of the canine subject and the second canine subject. In some embodiments, the social connection is generated when the expected range of pigmentation phenotypes of the potential offspring of the canine subject and the second canine subject includes a pre-determined pigmentation phenotype. In some embodiments, the social connection is generated through a social media network. In some embodiments, the first person is a pet owner of the first canine subject, and wherein the second person is a pet owner of the second canine subject.

In some embodiments, the method further comprises identifying the canine subject as having the predicted pigmentation phenotype among at least 3 different categorical or quantitative values of pigmentation phenotypes. In some embodiments, the method further comprises identifying the canine subject as having the predicted pigmentation phenotype among at least 6 different categorical or quantitative values of pigmentation phenotypes. In some embodiments, the method further comprises identifying the canine subject as having the predicted pigmentation phenotype with an accuracy of at least about 75%. In some embodiments, the method further comprises identifying the canine subject as having the predicted pigmentation phenotype with an accuracy of at least about 80%. In some embodiments, the method further comprises identifying the canine subject as having the predicted pigmentation phenotype among 2 different categorical or quantitative values of pigmentation phenotypes. In some embodiments, the 2 different categorical or quantitative values of pigmentation phenotypes comprise a darker coat color and a lighter coat color. In some embodiments, the method further comprises identifying the canine subject as having the predicted pigmentation phenotype with an accuracy of at least about 85%. In some embodiments, the method further comprises identifying the canine subject as having the predicted pigmentation phenotype with an accuracy of at least about 90%. In some embodiments, the method further comprises identifying the canine subject as having the predicted pigmentation phenotype with an accuracy of at least about 95%.

Additionally, methods and systems of the present disclosure may be used to add a valuable social component to the genetic assay results of dogs. By allowing dog owners to directly connect with each other based on a similarity of pigmentation of their pets, owners can gain more information from other dogs' owners about the suitability of a potential mating pairing between two dogs (e.g., having desired pigmentation traits).

Methods and systems of the present disclosure may use one or more algorithms to determine a pigmentation phenotype of a canine subject. In some embodiments, the canine subject is a dog. In some embodiments, the dog comprises one or more dog breeds selected from the group consisting of: Affenpinscher, Afghan Hound, Africanis, Aidi, Airedale Terrier, Akbash Dog, Akita Inu, Alangu Mastiff, Alano Español, Alapaha Blue Blood Bulldog, Alaskan Klee Kai, Alaskan Malamute, Alaunt, Alopekis, Alpine Dachsbracke, Alsatian Shepalute, American Akita, American Bulldog, American Cocker Spaniel, American Eskimo Dog, American Foxhound, American Hairless Terrier, American Mastiff, American Pit Bull Terrier, American Staffordshire Terrier, American Water Spaniel, Anatolian Shepherd Dog, Anglo-Français de Petite Vénerie, Appenzeller Sennenhund, Argentine Dogo, Ariege Pointer, Ariegeois, Armant, Artois Hound, Australian Bulldog, Australian Cattle Dog, Australian Kelpie, Australian Shepherd, Australian Silky Terrier, Australian Stumpy Tail Cattle Dog, Australian Terrier, Austrian Black and Tan Hound, Austrian Pinscher, Azawakh, Bakharwal Dog, Barbet, Basenji, Basque Shepherd Dog, Basset Artésien Normand, Basset Bleu de Gascogne, Basset Fauve de Bretagne, Grand Basset Griffon Vendéen, Petit Basset Griffon Vendéen, Bavarian Mountain Hound, Beagle, Beagle-Harrier, Bearded Collie, Beauceron, Bedlington Terrier, Belgian Shepherd Dog, Belgian Shepherd Dog (Groenendael), Belgian Shepherd Dog (Laekenois), Belgian Shepherd Dog (Malinois), Belgian Shepherd (Tervuren), Bergamasco Shepherd, Berger Blanc Suisse, Berger Picard, Berner Laufhund, Bernese Mountain Dog, Bichon Frisé, Billy, Bisben, Black and Tan Coonhound, Black and Tan Virginia Foxhound, Bullenbeisser, Black Norwegian Elkhound, Black Russian Terrier, Blackmouth Cur, Grand Bleu de Gascogne, Petit Bleu de Gascogne, Bloodhound, Blue Lacy, Blue Paul Terrier, Bluetick Coonhound, Boerboel, Bohemian Shepherd, Bolognese, Border Collie, Border Terrier, Borzoi, Bosnian Coarse-haired Hound, Boston Terrier, Bouvier des Ardennes, Bouvier des Flandres, Boxer, Boykin Spaniel, Bracco Italiano, Braque d'Auvergne, Braque du Bourbonnais, Braque du Puy, Braque Francais, Braque Saint-Germain, Brazilian Terrier, Briard, Briquet Griffon Vendéen, Brittany, Broholmer, Bruno Jura Hound, Bucovina Shepherd Dog, Bull and Terrier, Bull Terrier, Bull Terrier (Miniature), Bullmastiff, Bully Kutta, Cairn Terrier, Canaan Dog, Canadian Eskimo Dog, Canadian Pointer, Cane Corso, Cão da Serra de Aires, Cão de Castro Laboreiro, Cão Fila de Sao Miguel, Carolina Dog, Carpathian Shepherd Dog, Catahoula Cur, Catalan Sheepdog, Caucasian Shepherd Dog, Cavalier King Charles Spaniel, Central Asian Shepherd Dog, Cesky Fousek, Cesky Terrier, Polish Greyhound, Chesapeake Bay Retriever, Chien-gris, Chien Français Blanc et Noir, Chien Français Blanc et Orange, Chien Français Tricolore, Chihuahua, Chilean Fox Terrier, Chinese Chongqing Dog, Chinese Crested Dog, Chinese Imperial Dog, Chinook, Chippiparai, Chow Chow, Cimarrón Uruguayo, Cierny Sery, Cirneco dell'Etna, Clumber Spaniel, Rough Collie, Smooth Collie, Combai, Cordoba Fighting Dog, Coton de Tulear, Cretan Hound, Croatian Sheepdog, Cumberland Sheepdog, Curly Coated Retriever, Czechoslovakian Wolfdog, Dachshund, Dalmatian, Dandie Dinmont Terrier, Danish Swedish Farmdog, Dingo, Doberman Pinscher, Dogue de Bordeaux, Dogo Cubano, Dogo Guatemalteco, Dogo Sardesco, Drentse Patrijshond, Dreyer, Dunker, Dutch Shepherd Dog, Dutch Smoushond, East-European Shepherd, East Siberian Laika, Elo, English Cocker Spaniel, English Coonhound, English Foxhound, English Mastiff, English Pointer, English Setter, English Shepherd, English Springer Spaniel, English Toy Terrier (Black & Tan), English Water Spaniel, English White Terrier, Entlebucher Mountain Dog, Épagneul Bleu de Picardie, Estonian Hound, Estrela Mountain Dog, Eurasier, Field Spaniel, Fila Brasileiro, Findo, Finnish Hound, Finnish Lapphund, Finnish Spitz, Flat-Coated Retriever, Formosan Mountain Dog, Fox Terrier (Smooth), Wire Fox Terrier, French Brittany, French Bulldog, French Spaniel, Galgo Español, German Longhaired Pointer, German Pinscher, German Shepherd Dog, German Shorthaired Pointer, German Spaniel, German Spitz, German Wirehaired Pointer, Giant Schnauzer, Glen of Imaal Terrier, Golden Retriever, Gordon Setter, Grand Anglo-Français Blanc et Noir, Grand Anglo-Français Blanc et Orange, Grand Anglo-Français Tricolore, Grand Griffon Vendéen, Gran Mastin de Borinquen, Great Dane, Great Pyrenees, Greater Swiss Mountain Dog, Greenland Dog, Greyhound, Griffon Bleu de Gascogne, Griffon Bruxellois, Griffon Fauve de Bretagne, Griffon Nivernais, Gull Dong, Gull Terr, Hare Indian Dog, Hamiltonstövare, Hanover Hound, Harrier, Havanese, Hawaiian Poi Dog, Himalayan Sheepdog, Hokkaido, Hortaya Borzaya, Hovawart, Hungarian Hound, New Zealand Huntaway, Hygenhund, Ibizan Hound, Icelandic Sheepdog, Indian Spitz, Irish Bull Terrier, Irish Red and White Setter, Irish Setter, Irish Staffordshire Bull Terrier, Irish Terrier, Irish Water Spaniel, Irish Wolfhound, Istrian Shorthaired Hound, Istrian Coarse-haired Hound, Italian Greyhound, Jack Russell Terrier, Jagdterrier, Jämthund, Japanese Chin, Japanese Spitz, Japanese Terrier, Jonangi, Kaikadi, Kai Ken, Kangal Dog, Kanni, Karakachan Dog, Karelian Bear Dog, Karst Shepherd, Keeshond, Kerry Beagle, Kerry Blue Terrier, King Charles Spaniel, King Shepherd, Kintamani, Kishu, Komondor, Kooikerhondje, Koolie, Korean Jindo Dog, Korean Mastiff, Kromfohrlander, Kunming Wolf-dog, Kuri, Kuvasz, Kyi-Leo, Labrador Husky, Labrador Retriever, Lagotto Romagnolo, Lakeland Terrier, Lancashire Heeler, Landseer, Lapponian Herder, Leonberger, Lhasa Apso, Lithuanian Hound, Longhaired Whippet, Lottatore Brindisino, Lowchen, Magyar Agar, Majestic Tree Hound, Maltese, Manchester Terrier, Maremma Sheepdog, McNab, Mexican Hairless Dog, Miniature Australian Shepherd, Miniature Fox Terrier, Miniature Pinscher, Miniature Schnauzer, Miniature Siberian Husky, Mioritic, Molossus, Montenegrin Mountain Hound, Moscow Watchdog, Moscow Water Dog, Mountain Cur, Mountain View Cur, Mucuchies, Mudi, Mudhol Hound, Large Munsterlander, Small Munsterlander, Murray River Curly Coated Retriever, Neapolitan Mastiff, Newfoundland, New Guinea Singing Dog, Norfolk Spaniel, Norfolk Terrier, Norrbottenspets, North Country Beagle, Northern Inuit Dog, Norwegian Buhund, Norwegian Elkhound, Norwegian Lundehund, Norwich Terrier, Nova Scotia Duck-Tolling Retriever, Old Danish Pointer, Old English Sheepdog, Old English Bulldog, Old English Terrier, Old German Shepherd Dog, Olde English Bulldogge, Otterhound, Pachon Navarro, Paisley Terrier, Papillon, Parson Russell Terrier, Patterdale Terrier, Pekingese, Perro de Presa Canario, Perro de Presa Mallorquin, Peruvian Hairless Dog, Phaléne, Pharaoh Hound, Picardy Spaniel, Plott Hound, Podenco Canario, Pointer, Polish Hound, Polish Hunting Dog, Polish Lowland Sheepdog, Polish Tatra Sheepdog, Pomeranian, Pont-Audemer Spaniel, Poodle, Porcelaine, Portuguese Podengo, Portuguese Pointer, Portuguese Water Dog, Pražský Krysarik, Pudelpointer, Pug, Puli, Pumi, Pungsan Dog, Pyrenean Mastiff, Pyrenean Shepherd, Rafeiro do Alentejo, Rajapalayam, Rampur Greyhound, Rastreador Brasileiro, Ratonero Bodeguero Andaluz, Rat Terrier, Redbone Coonhound, Rhodesian Ridgeback, Rottweiler, Russian Spaniel, Russkiy Toy, Russo-European Laika, Russell Terrier, Saarlooswolfhond, Sabueso Español, Sage Ashayeri, Sage Mazandarani, Sakhalin Husky, Saluki, Samoyed, Sapsali, arplaninac, Schapendoes, Schillerstovare, Schipperke, Old Croatian Sighthound, Giant Schnauzer, Miniature Schnauzer, Standard Schnauzer, Schweizer Laufhund, Schweizerischer Niederlaufhund, Scotch Collie, Scottish Deerhound, Scottish Terrier, Sealyham Terrier, Segugio Italiano, Seppala Siberian Sleddog, Serbian Hound, Serbian Tricolour Hound, Shar Pei, Shetland Sheepdog, Shiba Inu, Shih Tzu, Shikoku, Shiloh Shepherd Dog, Shirak, Siberian Husky, Silken Windhound, Sinhala Hound, Skye Terrier, Sloughi, Slovak Cuvac, Slovakian Rough-haired Pointer, Slovenský Kopov, Smálandsstovare, Small Greek Domestic Dog, Soft-Coated Wheaten Terrier, South Russian Ovcharka, Southern Hound, Spanish Mastiff, Spanish Water Dog, Spinone Italiano, Sporting Lucas Terrier, St. Bernard, St. John's Water Dog, Stabyhoun, Staffordshire Bull Terrier, Stephens Cur, Styrian Coarse-haired Hound, Sussex Spaniel, Swedish Lapphund, Swedish Vallhund, Swedish Beagle, Tahltan Bear Dog, Taigan, Tamaskan Dog, Teddy Roosevelt Terrier, Telomian, Tenterfield Terrier, Thai Bangkaew Dog, Thai Ridgeback, Tibetan Mastiff, Tibetan Spaniel, Tibetan Terrier, Tornjak, Tosa, Toy Bulldog, Toy Fox Terrier, Toy Manchester Terrier, Treeing Cur, Treeing Walker Coonhound, Tyrolean Hound, Utonagan, Vizsla, Volpino Italiano, Weimaraner, Cardigan Welsh Corgi, Pembroke Welsh Corgi, Welsh Sheepdog, Welsh Springer Spaniel, Welsh Terrier, West Highland White Terrier, West Siberian Laika, Westphalian Dachsbracke, Wetterhoun, Whippet, White English Bulldog, White Shepherd Dog, Wirehaired Vizsla, Wirehaired Pointing Griffon, and Yorkshire Terrier. In some embodiments, the subject is a purebred dogs (e.g., having a single breed type) or a mixed-breed dog (e.g., having a plurality of breed types). In some embodiments, the subject is a mixed-breed dog having DNA from any number (e.g., 1, 2, 3, 4, 5, 6, 7, 8, 9, 10, or more than 10) or combination of purebred dogs.

The method may comprise receiving genotype data as inputs. For example, the genotype data may be obtained by assaying biological samples obtained from the population of test individuals. In some embodiments, the biological samples comprise blood samples, saliva samples, swab samples, cell samples (e.g., mouth or cheek swab), or tissue samples. In some embodiments, the assaying comprises sequencing the biological samples or derivatives thereof to generate the genotype data. For example, sequencing reads may be generated from the biological samples using any suitable sequencing method. The sequencing method can be a first-generation sequencing method, such as Maxam-Gilbert or Sanger sequencing, or a high-throughput sequencing (e.g., next-generation sequencing or NGS) method. A high-throughput sequencing method may sequence simultaneously (or substantially simultaneously) at least about 10,000, 100,000, 1 million, 10 million, 100 million, 1 billion, or more polynucleotide molecules. Sequencing methods may include, but are not limited to: pyrosequencing, sequencing-by-synthesis, single-molecule sequencing, nanopore sequencing, semiconductor sequencing, sequencing-by-ligation, sequencing-by-hybridization, Digital Gene Expression (Helicos), massively parallel sequencing, e.g., Helicos, Clonal Single Molecule Array (Solexa/Illumina), sequencing using PacBio, SOLiD, Ion Torrent, or Nanopore platforms.

In some embodiments, the sequencing comprises whole genome sequencing (WGS). The sequencing may be performed at a depth sufficient to generate the desired genotype data with a desired performance (e.g., accuracy, sensitivity, specificity, positive predictive value (PPV), negative predictive value (NPV), or the area under curve (AUC) of a receiver operator characteristic (ROC)). In some embodiments, the sequencing is performed at a depth of about 20×, about 30×, about 40×, about 50×, about 60×, about 70×, about 80×, about 90×, about 100×, about 150×, about 200×, about 250×, about 300×, about 350×, about 400×, about 450×, about 500×, or more than about 500×. In some embodiments, the sequencing is performed in a “low-pass” manner, for example, at a depth of no more than about 12×, no more than about 11×, no more than about 10×, no more than about 9×, no more than about 8×, no more than about 7×, no more than about 6×, no more than about 5×, no more than about 4×, no more than about 3.5×, no more than about 3×, no more than about 2.5×, no more than about 2×, no more than about 1.5×, or no more than about 1×.

In some embodiments, the sequencing reads may be aligned to a reference genome. The reference genome may comprise at least a portion of a genome (e.g., a dog genome or a human genome). The reference genome may comprise an entire genome (e.g., an entire dog genome or an entire human genome). The reference genome may comprise a database comprising a plurality of genomic regions that correspond to coding and/or non-coding genomic regions of a genome. The database may comprise a plurality of genomic regions that correspond to coding and/or non-coding genomic regions of a genome, such as single nucleotide variants (SNVs), single nucleotide polymorphisms (SNPs), copy number variants (CNVs), insertions or deletions (indels), and fusion genes. The alignment may be performed using a Burrows-Wheeler algorithm or another alignment algorithm.

In some embodiments, quantitative measures of the sequencing reads may be generated for each of a plurality of genomic regions. Quantitative measures of the sequencing reads may be generated, such as counts of DNA sequencing reads that are aligned with a given genomic region. Sequencing reads having a portion or all of the sequencing read aligning with a given genomic region may be counted toward the quantitative measure for that genomic region. In some embodiments, genomic regions may comprise genetic markers such as genetic variants (e.g., single nucleotide polymorphisms (SNPs), insertions or deletions (indels), microsatellites, or structural variants). Patterns of specific and non-specific genomic regions may be indicative of pigmentation phenotypes (e.g., color coat intensity, roaning, ticking, or tongue pigmentation).

In some embodiments, measuring the plurality of counts of DNA sequencing reads comprises performing binding measurements of the plurality of DNA molecules at each of the plurality of genomic regions. In some embodiments, performing the binding measurements comprises assaying the plurality of DNA molecules using probes that are selective for at least a portion of the plurality of genomic regions in the plurality of DNA molecules. In some embodiments, the probes are nucleic acid molecules having sequence complementarity with nucleic acid sequences of the plurality of genomic regions. In some embodiments, the nucleic acid molecules are primers or enrichment sequences. In some embodiments, the assaying comprises use of array hybridization or polymerase chain reaction (PCR), or nucleic acid sequencing.

In some embodiments, the method further comprises enriching the plurality of DNA molecules for at least a portion of the plurality of genomic regions. In some embodiments, the enrichment comprises amplifying the plurality of DNA molecules. For example, the plurality of DNA molecules may be amplified by selective amplification (e.g., by using a set of primers or probes comprising nucleic acid molecules having sequence complementarity with nucleic acid sequences of the plurality of genomic regions). Alternatively or in combination, the plurality of DNA molecules may be amplified by universal amplification (e.g., by using universal primers). In some embodiments, the enrichment comprises selectively isolating at least a portion (e.g., mononucleotides and/or dinucleotides) of the plurality of DNA molecules.

In some embodiments, the counts of DNA sequencing reads may be normalized or corrected. For example, the counts of DNA sequencing reads may be normalized and/or corrected to account for known biases in sequencing and library preparation and/or known biases in sequencing and library preparation. In some embodiments, a subset of the quantitative measures or counts may be filtered out, e.g., based on a quality score of the sequencing reads.

Trained Algorithms

After assaying a biological sample to generate genotype data from a canine subject, a trained algorithm (e.g., a machine learning classifier) may be used to analyze the genotype data to determine a predicted pigmentation phenotype of the canine subject. In some embodiments, the genotype data comprises quantitative values of each of a plurality of genetic markers (e.g., genetic variants). For example, the trained algorithm may be used to determine quantitative or categorical measures of a predicted pigmentation phenotype of the canine subject. The trained algorithm may be configured to determine the predicted pigmentation phenotype with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%.

The trained algorithm may comprise a supervised machine learning algorithm. The trained algorithm may comprise a classification and regression tree (CART) algorithm. The supervised machine learning algorithm may comprise, for example, a linear regression, a logistic regression, a Random Forest, a support vector machine (SVM), a neural network, or a deep learning algorithm. The trained algorithm may comprise an unsupervised machine learning algorithm.

The trained algorithm may be configured to accept a plurality of input variables and to produce one or more output values based on the plurality of input variables. The plurality of input variables may be generated based on processing genotype data of nucleic acids. For example, an input variable may comprise a number of sequences corresponding to or aligning to a reference genome or genomic loci of a reference genome. As another example, an input variable may comprise analog or digital values of genotype data produced by a sequencer or array.

The trained algorithm may comprise a classifier, such that each of the one or more output values comprises one of a fixed number of possible values (e.g., a linear classifier, a logistic regression classifier, etc.) indicating a classification of the genotype data by the classifier. The trained algorithm may comprise a binary classifier, such that each of the one or more output values comprises one of two values (e.g., {0, 1}, {positive, negative}, {present, absent}, or {light, dark}) indicating a classification of the canine subject based on genotype data by the classifier. The trained algorithm may be another type of classifier, such that each of the one or more output values comprises one of more than two values (e.g., {0, 1, 2}, {positive, negative, or indeterminate}, {present, absent, or indeterminate}, or {light, medium, or dark}) indicating a classification of the canine subject based on genotype data by the classifier. The output values may comprise descriptive labels, numerical values, or a combination thereof. Some of the output values may comprise descriptive labels. Such descriptive labels may provide an identification of predicted pigmentation phenotypes, and may comprise, for example, {light, medium, or dark}. As another example, such descriptive labels may provide a relative assessment of the likelihood of different pigmentation phenotypes being present in the canine subject based on the genotype data. Some descriptive labels may be mapped to numerical values, for example, by mapping “positive” or “present” to 1, and “negative” or “absent” to 0.

Some of the output values may comprise numerical values, such as binary, integer, or continuous values. Such binary output values may comprise, for example, {0, 1}, {positive, negative}, or {present, absent}. Such integer output values may comprise, for example, {0, 1, 2}. Such continuous output values may comprise, for example, a probability value of at least 0 and no more than 1 (e.g., indicative of the likelihood of different pigmentation phenotypes being present in the canine subject). Such continuous output values may comprise, for example, an un-normalized probability value of at least 0. Some numerical values may be mapped to descriptive labels, for example, by mapping 1 to “positive” or “present”, and 0 to “negative” or “absent”.

Some of the output values may be assigned based on one or more cutoff values. For example, a binary classification of the canine subject based on genotype data may assign an output value of “positive” or 1 if the canine subject has at least a 50% probability of having a given pigmentation phenotype. For example, a binary classification of the canine subject based on genotype data may assign an output value of “negative” or 0 if the canine subject has less than a 50% probability of having a given pigmentation phenotype. In this case, a single cutoff value of 50% is used to classify the canine subject into one of the two possible binary output values based on genotype data. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of the canine subject based on genotype data may assign an output value of “positive” or 1 if the canine subject has a probability of having a given pigmentation phenotype of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of the canine subject based on genotype data may assign an output value of “positive” or 1 if the canine subject has a probability of having a given pigmentation phenotype of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of genotype data may assign an output value of “negative” or 0 if the canine subject has a probability of having a given pigmentation phenotype of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of genotype data may assign an output value of “negative” or 0 if the canine subject has a probability of having a given pigmentation phenotype of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of the canine subject based on genotype data may assign an output value of “indeterminate” or 2 if the canine subject is not classified as “positive”, “negative”, 1, or 0. In this case, a set of two cutoff values is used to classify the canine subject based on genotype data into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify the canine subject based on genotype data into one of n+1 possible output values, where n is any positive integer.

The trained algorithm may be trained with a plurality of independent training samples. Each of the independent training samples may comprise sets of genotype data generated from nucleic acids (e.g., from a biological sample of a canine subject) and one or more known output values corresponding to the genotype data (e.g., a set of known pigmentation phenotypes corresponding to the genotype data, such as that generated from photographs of the canine subjects). Independent training samples may be obtained or derived from a plurality of different subjects. Independent training samples may comprise sets of genotype data generated from nucleic acids (e.g., from a biological sample of a canine subject) and one or more known output values corresponding to the genotype data (e.g., a set of known pigmentation phenotypes corresponding to the genotype data, such as that generated from photographs of the canine subjects) obtained at a plurality of different time points from the same subject.

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The trained algorithm may be trained with no more than about 500, no more than about 450, no more than about 400, no more than about 350, no more than about 300, no more than about 250, no more than about 200, no more than about 150, no more than about 100, or no more than about 50 independent training samples.

The trained algorithm may be configured to determine a predicted pigmentation phenotype based on genotype data at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. The accuracy of identifying a predicted pigmentation phenotype by the trained algorithm may be calculated as the percentage of canine subjects that are correctly identified or classified (e.g., presence or absence of a particular pigmentation phenotype).

The trained algorithm may be configured to identify predicted pigmentation phenotypes with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the predicted pigmentation phenotypes using the trained algorithm may be calculated as the percentage of pigmentation phenotypes identified or classified as being present that correspond to pigmentation phenotypes that are truly present.

The trained algorithm may be configured to identify predicted pigmentation phenotypes with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the predicted pigmentation phenotypes using the trained algorithm may be calculated as the percentage of pigmentation phenotypes identified or classified as being absent that correspond to pigmentation phenotypes that are truly absent (e.g., not present).

The trained algorithm may be adjusted or tuned to improve one or more of the performance, accuracy, PPV, or NPV of identifying the predicted pigmentation phenotypes. The trained algorithm may be adjusted or tuned by adjusting parameters of the trained algorithm (e.g., a set of cutoff values used to predict pigmentation phenotypes, as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. The plurality of input variables or a subset thereof may be ranked based on classification metrics indicative of each input variable's importance toward making high-quality classifications or identifications of pigmentation phenotypes. Such metrics may be used to reduce, in some cases significantly, the number of input variables (e.g., predictor variables) that may be used to train the trained algorithm to a desired performance level (e.g., based on a desired minimum accuracy, PPV, or NPV, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

Computer Systems

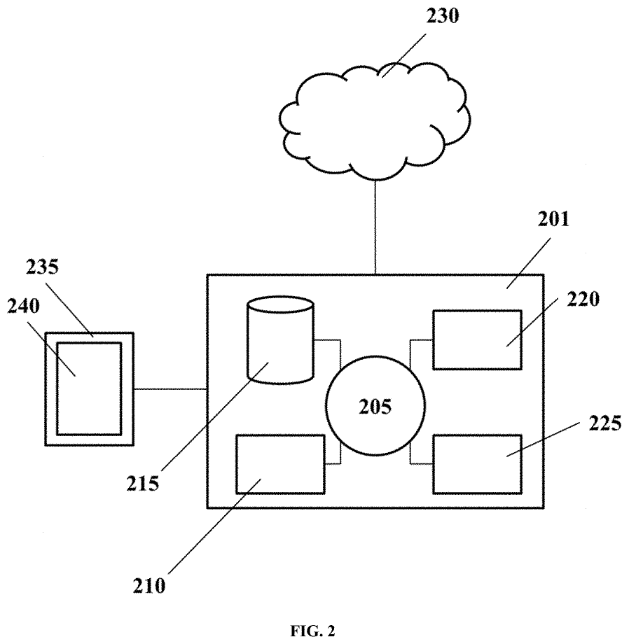

The present disclosure provides computer systems that are programmed to implement methods of the disclosure. FIG. 2 shows a computer system 201 that is programmed or otherwise configured to, for example, receive genotype data for a canine subject, apply a trained machine learning classifier to genotype data to determine a predicted pigmentation phenotype, and identify canine subjects as having the predicted pigmentation phenotype. The computer system 201 can regulate various aspects of analysis, calculation, and generation of the present disclosure, such as, for example, receiving genotype data for a canine subject, applying a trained machine learning classifier to genotype data to determine a predicted pigmentation phenotype, and identifying canine subjects as having the predicted pigmentation phenotype. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. For example, one or more computer servers may enable cloud computing over the network 230 (“the cloud”) to perform various aspects of analysis, calculation, and generation of the present disclosure, such as, for example, receiving genotype data for a canine subject, applying a trained machine learning classifier to genotype data to determine a predicted pigmentation phenotype, and identifying canine subjects as having the predicted pigmentation phenotype. Such cloud computing may be provided by cloud computing platforms such as, for example, Amazon Web Services (AWS), Microsoft Azure, Google Cloud Platform, and IBM cloud. The network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user (e.g., a pet owner, a kennel owner, a veterinarian, a breeder, an animal shelter employee, a physician, a nurse, a caretaker, a patient, or a subject). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, genotype data, genetic markers, quantitative values of genetic variants, and predicted pigmentation phenotypes. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, receive genotype data for a canine subject, apply a trained machine learning classifier to genotype data to determine a predicted pigmentation phenotype, and identify canine subjects as having the predicted pigmentation phenotype.

### Examples

### Example 1— Statistical Models for Prediction of Roaning Phenotypes in the Domestic Dog from Genetic Markers

Consumer genomics may enable genetic discovery on an unprecedented scale by linking very large databases of personal genomic data with phenotype information voluntarily submitted via web-based surveys. These databases may have a transformative effect on human genomics research, yielding insights on increasingly complex traits, behaviors, and disease by including many thousands of individuals in genome-wide association studies (GWAS). The promise of consumer genomic data may not be limited to human research, however. Genomic tools for canine subjects (e.g., dogs) may be readily available, with hundreds of causal Mendelian variants already characterized, because selection and breeding may lead to dramatic phenotypic diversity underlain by a simple genetic structure.

Results are reported of a consumer genomics study conducted in a non-human model: a GWAS of blue eyes based on more than 3,000 customer dogs with validation panels including nearly 3,000 more, a large canine GWAS study. A novel association was discovered with blue eyes on chromosome 18 (P=1.3×10⁻⁶⁸), and both sequence coverage and microarray probe intensity data were used to identify the putative causal variant: a 98.6-kb duplication directly upstream of the Homeobox gene ALX4, which may play an important role in mammalian eye development. This duplication is largely restricted to Siberian Huskies, is strongly associated with the blue-eyed phenotype (chi-square P=5.2×10⁻²⁹⁰), and is highly, but not completely, penetrant. These results underscore the power of consumer-data-driven discovery in non-human species, especially dogs, where there is intense owner interest in the personal genomic information of their pets, a high level of engagement with web-based surveys, and an underlying genetic architecture ideal for mapping studies.

Extensive variation in coat color and patterns in dogs, ranging from snowy white Samoyed to pitch black Labrador Retrievers and everything in-between, fascinates both dog enthusiasts and scientists who are interested in what drives such diversity. By taking advantage of publicly available whole-genome sequencing data and over 200,000 SNP data genotyped by a custom array, it was shown that dogs with roaned coat (mottled coat with a mixture of white and pigmented hairs) almost always carry a small duplication involving a part of USH2A gene on chromosome 38. The distinctive spots of Dalmatians were found to be likely formed by the same genetic variant, because all 247 Dalmatians that were examined had the duplication on chromosome 38.

Studies of the functional mechanism underlying this association may lead to discovery of pathways by which blue-eyes develop in mammals. These results highlight the power and promise of consumer-data-driven discovery in non-human species.

Diverse coat colors and patterns observed in dogs and other domestic animals have long fascinated breeders. Geneticists and evolutionary biologists have taken interest in this remarkable diversity because it provides an excellent model to study 1) how numerous phenotypes can be established from morphologically less diverse wild counterparts, 2) how changes in selection pressure alters genotype-phenotype interactions, and 3) how repeated evolution of similar coat phenotypes in multiple species is attainable. Years of research on the genetics of coat color have identified a number of genes that have been repeatedly involved in the formation of diverse color morphs in both wild and domesticated animals. One of the genes involved in the repeated evolution of light and dark color polymorphisms is the melanocortin-1 receptor (MC1R) that modulates the activity of the melanin synthesis pathways in the fur, plumage, scales and skin of mammals, birds, reptiles, amphibians, and fish. In other cases, similar coloration has independently evolved in multiple lineages via mutations in different genes (e.g., LYST and AIM1 in polar bears and KIT in horses with white coats). Understanding the genetic mechanisms of color variation and phenotypic convergence has shed light on how phenotypes evolve under similar selective forces (either natural or artificial). In addition, high conservation of melanogenesis pathways across vertebrates warrants transformative research in human genomics research, such as the case of MC1R that is strongly associated with risk of melanomas.

Ticking and roaning are two common coat patterns observed in dogs and other domestic animals. Ticking may be characterized as small pigmented spots of varying numbers and sizes appearing on otherwise unpigmented (white) areas. Roaning is similar to, and sometimes co-occurs with, ticking, but may include pigmented and unpigmented hairs interspersed more evenly without the formation of distinct spots. Typically, individuals are not immediately born with ticking and roaning patterns, but instead these pigmented areas may develop as the individual ages, indicating time-dependent action of underlying pigmentation mechanisms. In dogs, two theoretical loci, namely “T-locus” and “R-locus”, may be proposed as responsible loci for ticking and roaning, respectively, although it may be unclear whether they constitute the same genomic regions with different variants (characterization at a molecular level may be performed to establish this). KIT ligand gene (KITLG) may be a causal gene for roaning in cattle, horses, pigs, and goats. However, KITLG may not be involved in the formation of roaned coat in dogs based on segregation analysis results.

Gene interaction or epistasis is one of the key mechanisms in the formation of phenotypic diversity in both wild and domesticated species. An example is three color types of Labrador Retrievers, where tyrosinase-related protein 1 (TYRP1) and MC1R determine their coat colors as black, chocolate, or yellow. Modifier genes may constitute a type of epistasis; for example, several variants of microphthalmia-associated transcription factor (MITE) modify the coat color of dogs by preventing the melanocyte development and migration in certain areas of the body and, in some cases, across nearly the entire body. This results in a loss of pigmentation leading to white markings in otherwise uniformly colored areas, both by the T-locus and by locally changing coat color from white to pigmented. Furthermore, unique spots found exclusively in Dalmatians may be a result of an epistatic interaction between T-locus and a locus linked to huu locus on chromosome 3 (CFA3). To understand how these loci interact at a molecular level, the identification of causal variants may be performed, followed by thorough functional analyses, including gene expression difference and protein interactions.

Genomic regions associated with ticking and roaning coat patterns in dogs were investigated by using a total of 1,009 dogs that were genotyped at 228,830 markers covering all 38 autosomes and chromosome X. Dog owners contributed to this study by providing photographs of their dogs, from which their phenotypes were classified as ticked, roaned, or lacking these patterns, to identify genomic regions associated with these phenotypes by genome-wide association study (GWAS). For the discovery of the associated genomic regions, a diverse set of dogs were used from 27 breeds and their mixes with varying degrees of ticking and roaning patterns. In addition, publicly available whole-genome re-sequencing data was used to fine-map the location of a putative causal variant associated with roaning. The genotype-phenotype association was robust, as phenotypes of the independent validation dataset was accurately predicted by their genotypes. Finally, the unique “spotted” phenotype found in Dalmatians may be observed a result of epistatic interaction between roaning “R-locus” and a modifier locus.

Results were obtained as follows. A novel association was observed on chromosome 38 with roaning (but not with ticking). Further, a 11-kilobase tandem duplication was identified. Further, phenotype and genotype association was performed. Further, prediction of roaning coat pattern in an 888-dog validation panel was performed. Further, selection on the CFA38 was performed. Further, functional annotation was performed. Further, genotyping and genome-wide association was performed. Further, identification of tandem duplication was performed. Further, identification of tandem duplication was performed. Further, signatures of selection were detected.

Table 1 shows the number of dogs and breeds used for genome-wide association study. A total of 1,099 dogs with profile pictures from a database were used by targeting 27 breeds and their mixes (1,000 and 99 purebred and mixed dogs, respectively) (Table 1). The manual curation of customer-provided photographs identified 332 dogs with varying degrees of ticking, 320 dogs with a clear roaning pattern on some part of the body, and 357 dogs without any noticeable ticking and roaning in any part of their bodies. Dogs that exhibited both phenotypes (ticking and roaning) were not used in the subsequent analyses (N=90).

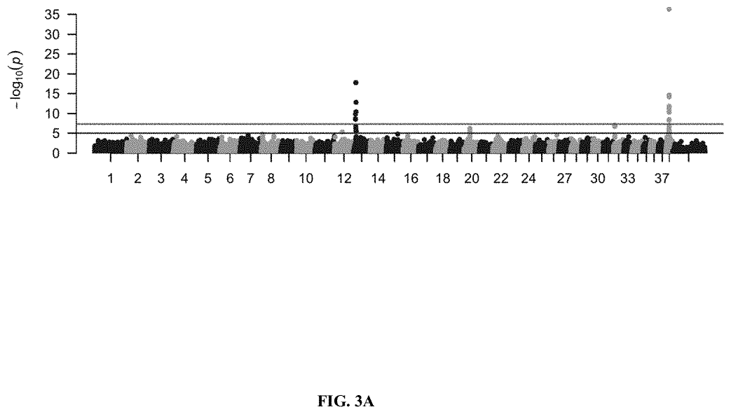

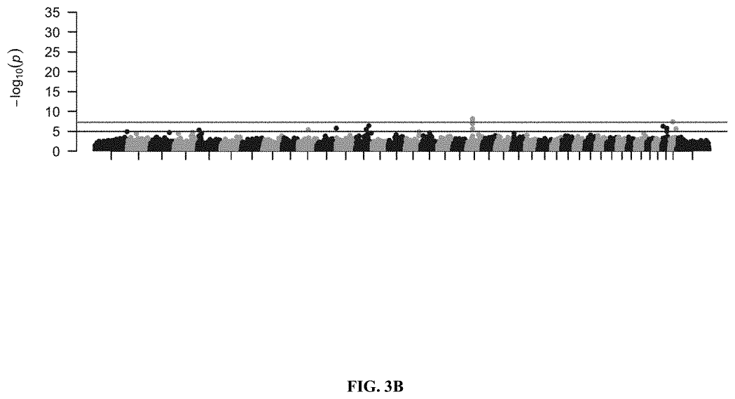

FIGS. 3A-3B show Manhattan plots of association with roaning (FIG. 3A) and ticking (FIG. 3B). Red and blue horizontal lines are significant (P<5×10⁻³⁷) and suggestive (P<1×10⁻⁵) associations, respectively.

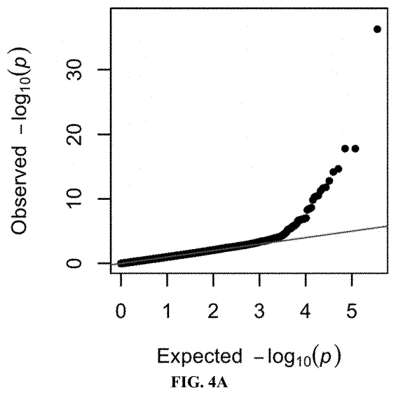

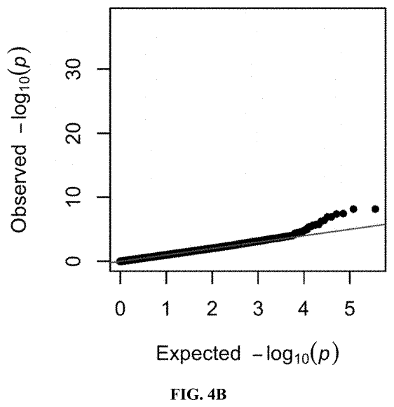

FIGS. 4A-4B show a Q-Q plot of the association with roaning (FIG. 4A) and ticking (FIG. 4B). The GWAS of 320 roaned dogs (cases) and 357 non-ticked, non-roaned dogs (controls) identified two highly significant and two suggestive markers (FIG. 3A and FIGS. 4A-4B). First, the most significant marker was at the position 11,085,443 on CFA38, an exonic region of usherin gene (USH2A) (P=3.8×10⁻³⁷). The second most significant marker overlapped with R-spondin 2 gene (RSPO2) on CFA13 at the position 8,625,896 (P=1.4×10⁻¹⁸). The associations with RSPO2 likely resulted from the breeds with contrasting coat texture, such as Border Collies and German Wirehaired Pointers, due to the association between this gene and wiry texture of the fur. Similarly, the association with the marker on CFA32 at the position 4,509,367 (P=8.7×10⁻⁸) was likely due to the effect of fibroblast growth factor 5 (FGF5) that may be associated with fur length, such as Border Collies and Australian Cattle Dogs. Further, the fourth suggestive marker was found at the position 21,836,232 on CFA20, an upstream of MITF (P=5.5×10⁻⁷). Pigmented fur may be visible in a white background (e.g., coat patterns known as Irish spotting, piebald, or extreme white), which was likely formed by MITF variants. To minimize the effect of genes associated with breed-specific characteristics, GWAS was re-run by subdividing the dataset by herding breeds (Australian Cattle Dogs, Australian Shepherds, and Border Collies) and the rest of breeds (hereafter referred to as non-herding breeds).

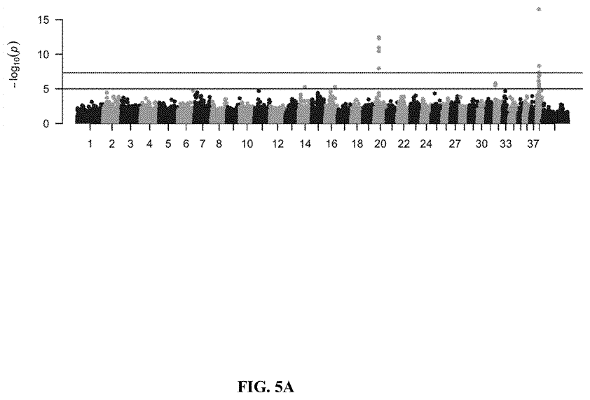

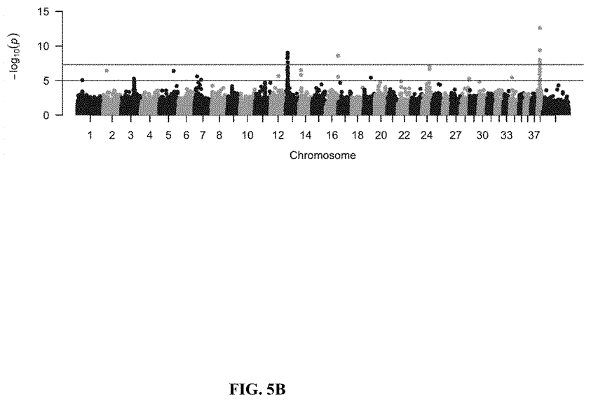

FIGS. 5A-5B show Manhattan plots of association with roaning, including roaning for herding breeds (FIG. 5A) and roaning for non-herding breeds (FIG. 5B). Red and blue horizontal lines are significant (P<5×10⁻⁸) and suggestive (P<1×10⁻⁵) associations, respectively. As expected, the GWAS association signals on CFA13, CFA20, and CFA32 were not consistently detected in GWAS for herding (N=235 and 223 for roaned and control dogs, respectively) and non-herding breeds (N=85 and 134 for roaned and control dogs, respectively) (FIG. S2). On the other hand, the marker on CFA38 remained highly significant in both herding and non-herding groups (P=2.6×10⁻¹⁷ and P=1.5×10⁻¹³, respectively). The non-roaned control group was devoid of the roaning-allele (A) at the CFA38 marker, while the frequency of this allele was 72% and 65% in roaned herding and working dogs, respectively.

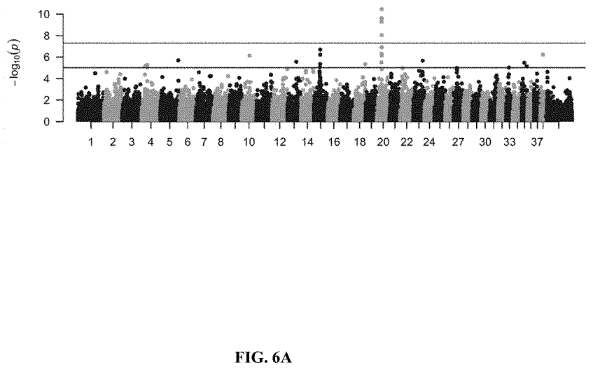

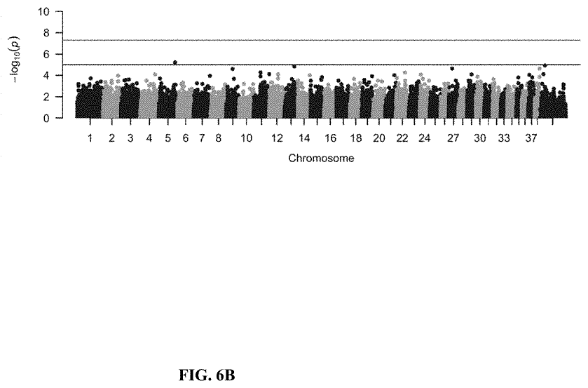

FIGS. 6A-6B show Manhattan plots of association with ticking, including ticking for herding breeds (FIG. 6A) and ticking for non-herding breeds (FIG. 6B). Red and blue horizontal lines are significant (P<5×10⁻⁸) and suggestive (P<1×10⁻⁵) associations, respectively. Contrasting to the GWAS for roaning, the GWAS for the ticking pattern did not reveal strongly associated markers (N=332 for cases, and N=357 for controls) (FIG. 3B and FIGS. 4A-4B). The same markers at MITF on CFA20 and USH2A on CFA38 were detected as marginally significant when both herding and non-herding breeds were analyzed together (P=1.0×10′ and P=4.0×10⁻⁸, respectively). However, when herding and non-herding breeds were analyzed separately, only the MITF marker was significant in herding breeds (N=84 for ticked dogs, and N=268 for control dogs), whereas no marker was significantly associated with ticking in non-herding breeds (N=248 for cases, and N=89 for controls) (FIGS. 6A-6B).

Further, a 11-kilobase tandem duplication was identified as follows. The association between the marker on CFA38 and roaning coat phenotype was robust regardless of whether herding or non-herding breeds were used, whereas this marker was not strongly associated with ticking. The analysis was focused on the region surrounding this marker to further characterize the genetic variants associated with roaning. It was observed that the mean probe intensity of the Illumina microarray (log R ratio: LRR) was significantly higher in roaned dogs than control dogs at a marker position 11,140,991 on CFA38, 55-kb away from the most significant GWAS marker (probe intensity=0.154 and −0.021, N=249 for roaned dogs, and N=208 for control dogs; t-test, t=−19.657, P<10⁻¹⁶). Stronger probe intensity indicated the existence of a duplicated copy of this region. There was no difference in the probe intensity at this marker between ticked and control dogs (−0.018 and −0.021, N=55 for ticked dogs, and N=208 for control dogs; t-test, t=−0.858, P=0.39).

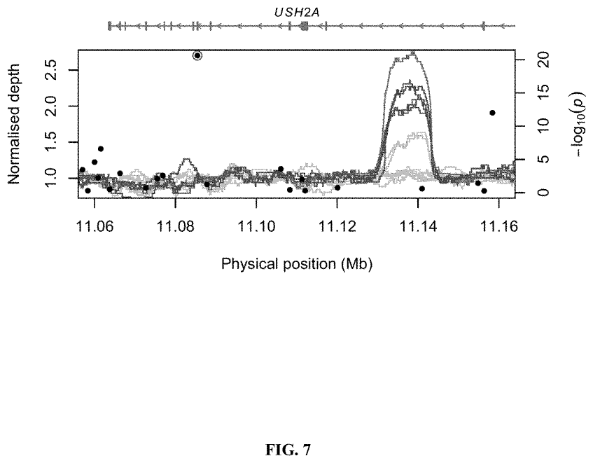

FIG. 7 shows normalized read depth in 5-kb sliding windows across the significant GWAS locus on CFA38 for Australian Cattle Dogs (red), German Wirehaired Pointer (pink), and Border Collies (grey). Filled circles shows the corresponding region of the Manhattan plot shown in FIGS. 3A-3B. Note that two Border Collies were heterozygous in the duplication showing the elevated depth.

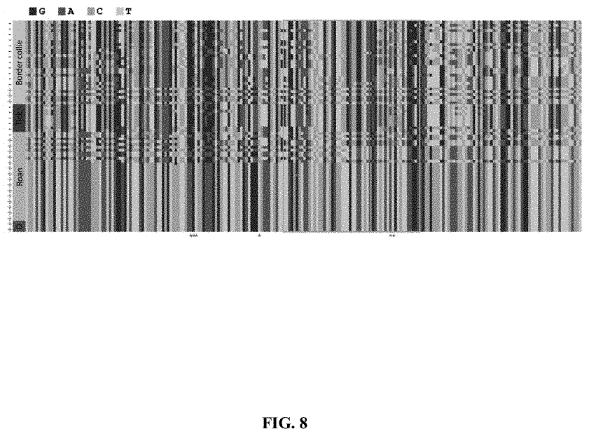

FIG. 8 shows haplotype structure near the tandem duplication on CFA38 (position 11,031,835-11,243,237). Border Collies (grey), breeds with high frequency of ticking (Brittany, Clumber spaniel, and English setter; purple), breeds with high frequency of roaning (Australian Cattle Dog, German Wirehaired Pointer, Wirehaired Pointer, and Wirehaired Pointing Griffon; brown), and Dalmatians (red). Rows correspond to haplotypes (two rows/individual), and columns correspond to markers. +/−: presence and absence of the 11-kb duplication based on Manta. Red box: 11-kb duplication (CFA38:11,131,835-11,143,234). Orange box: a core haplotype (CFA38:11,122,646-11,167,876).

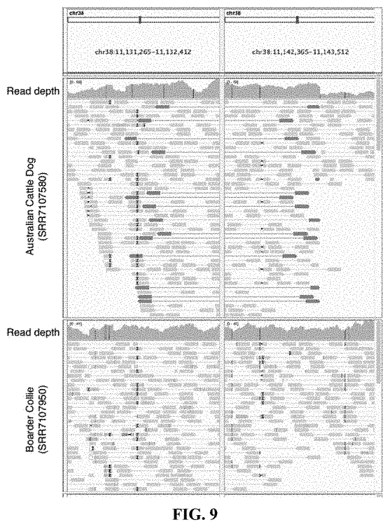

Table 2 shows whole genome re-sequencing data used for characterizing the 11-kb tandem duplication on CFA38. FIG. 9 shows discordant read pairs at the duplication breakpoint on CFA38 identified in Münsterländer (top panel), Australian Cattle Dog (middle: SRR7107580), and Border Collie (bottom: SRR7107950). Outward-facing read pairs (green) indicate that this is a tandem duplication found in ticked and roaned dogs but not in Border Collie. To identify a duplication associated with roaning, structural variations (SVs) were searched by using publicly available whole genome re-sequencing data (Table 2). Manta, an SV caller based on short-read sequence data, was used to identify a 11.4-kb duplication (CFA38:11,131,835-11,143,237) in 15 out of 16 dogs of breeds that may be associated with roaned coat (4 Australian Cattle Dog, 1 German Wirehaired Pointer, 1 Wirehaired Pointer, and 11 Wirehaired Pointing Griffon). Five dogs in three breeds that are often associated with ticking patterns did not carry the duplication (1 Brittany, 1 Clumber Spaniel, and 3 English Setter), while 3 out of 15 Border Collies carried the duplication (Table 2). Read depth of the dogs with the duplication was 1.5-2.5 times higher in regions between 11.13 Mb and 11.14 Mb than the genome-wide average (FIG. 7). Border Collies did not show such an abrupt increase except for the three dogs where the tandem duplication was identified by Manta. Discordant read-pairs facing outwards were observed in dogs with elevated read depth in the duplicated region, suggesting that the duplication was in a tandem orientation (FIG. 9). Out of 15 potentially roaned dogs with the duplication, 10 dogs were homozygous at the most significant GWAS marker on CFA38 and shared a long haplotype 100-kb upstream and downstream of the duplication (hereafter referred to as the duplication-associated haplotype) (FIG. 8). Three dogs were heterozygous at the most significant GWAS marker on CFA38 and had one copy of the duplication-associated haplotype. Two dogs were homozygous of the alternate allele at the most significant GWAS marker on CFA38 and did not have the duplication-associated haplotype. However, one of their chromosomes was a likely recombinant by sharing a core haplotype from the positions 11,122,646-11,167,876 that was identical to the duplication-associated haplotype. The dogs without the duplication did not have the duplication-associated haplotype or similar ones.

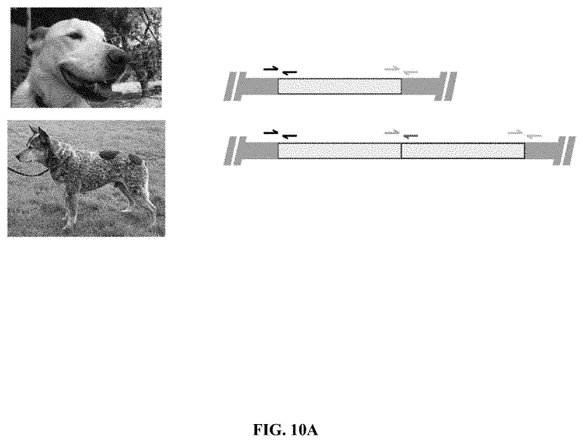

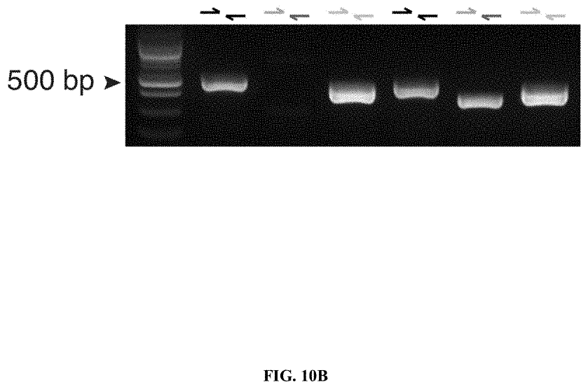

FIGS. 10A-10B show PCR genotyping of the tandem duplication on CFA38 associated with roaning. FIG. 10A shows a schematic view of the design of the PCR genotyping assay. Single headed arrows indicate three pairs of primers to amplify three regions. The first (black) and the third (yellow) primer pairs should produce amplicons in all dogs regardless of the presence or absence of the duplication, while the second pair in the middle should produce an amplicon only in dogs carrying the duplication. FIG. 10B shows PCR genotyping of a roaned and control dogs. Each gel lane corresponds to PCR primer pairs depicted in FIG. 10A.

Table 3 shows (a) primer sequences used for PCR assays described in FIG. 8. (b) Midpoint span product sequence. Nucleotides in bold and italic are likely the end of the first copy and the beginning of the second copy, respectively. To test whether the duplication-associated haplotype identified in the whole genome re-sequence data was indeed associated with the tandem duplication in the discovery panel dogs, a breakpoint PCR assay was designed by targeting the region spanning the two copies (forward and reverse primers mapping to CanFam3.1 CFA38:11,143,136-11,143,155 and CFA38:11,131,969-11,131,988, respectively) (FIGS. 10A-10B, Table 3). A total of 99 dogs (73 with roaning and 26 dogs without roaning) were assayed. This primer pair produced a single 400-bp amplicon in 64 dogs (FIGS. 10A-10B). To define haplotypes, six markers were used in the flanking region of the duplication, including the most significant GWAS marker (FIG. 8). About 83% of the PCR-positive dogs (54 dogs) carried at least one copy of the duplication-associated haplotype (hap-1: “AGAGGA”). Six dogs (9%) had at least one copy of the recombinant haplotype identified in the whole genome re-sequencing data (hap-2: “GGAGGA”). A third haplotype associated with the duplication was identified in two dogs (3%), one with one copy and the other with two copies of this haplotype (hap-3: “GAAAAA”).

There were 5,422 purebred dogs in the database that had at least one copy of the duplication-associated “AGAGAA” hap-1 (9%). Apart from the target breeds used in the GWAS (Table 1), this haplotype was nearly fixed in Dalmatians, where 98% (242 dogs) had at least one copy of the duplication-associated haplotype (208 homozygous dogs and 34 heterozygous dogs). The high frequency of the CFA38 duplication in Dalmatians was confirmed by three independent analyses. First, the breakpoint PCR assay showed that all three Dalmatians with hap-1 that were tested indeed had the duplication. Second, the whole-genome variant analysis showed that the haplotypes of the two Dalmatians were identical to the duplication-associated haplotype identified in dogs that are typically associated with roaning in the region 11,031,835-11,243,237 on CFA38 (FIG. 8, Table 2). Finally, Manta analysis was used to detect the tandem duplication in the corresponding region in both of these two dogs. In addition, the remaining 2% of Dalmatians in the database (N=5) were homozygous for a haplotype “GAGGAA” (hap-4). This haplotype was confirmed to contain the duplication by the breakpoint PCR assay by using available DNA samples (N=3).

Further, phenotype and genotype association was performed as follows. Table 4 shows genotype frequencies of the markers associated with roaning in the discovery panel, including A) CFA38 Duplication and B) The top associated GWAS SNP. The presence or absence of the tandem duplication on CFA38 was predicted for the discovery panel dogs based on the three haplotypes associated with the duplication (hap-1, hap-2, and hap-3). A total of 404 dogs had at least one copy of the duplication-associated haplotypes. The presence of the duplication-associated haplotypes explained 95% of roaned cases (211 homozygous and 106 heterozygous dogs out of 320 roaned dogs), whereas these haplotypes were extremely rare in non-roaned dogs where only one heterozygous dog with the hap-2 was found in the discovery panel (0.3%). These duplication-associated haplotypes may predict the roaned coat pattern more accurately than the genotypes of the most associated SNP in the GWAS (chi-squared test, P=6.4×10⁻¹⁴⁵ for the duplication and P=2.9×10⁻¹³⁴ for the GWAS marker) (Table 4). Only 5% of ticked dogs had one copy of the duplication-associated haplotypes (16 heterozygotes and no duplication homozygote). Although the association between ticking and the duplication was significant (chi-squared test, P=3.3×10⁻⁴), this observation may be due to cryptic roaned fur that was not visible in the photographs.

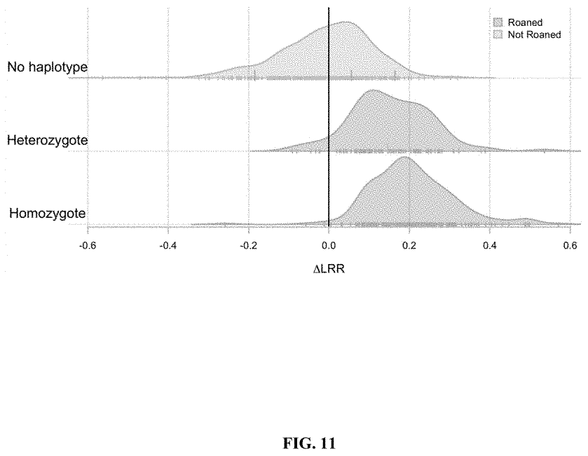

To confirm that the roaned dogs carried the CFA38 tandem duplication, the difference in LRR between a marker located within the duplication (the position 11,140,991) and 10 markers in the flanking region of the duplication (ΔLRR=LRR_{inside}−LRR_{outside}) was compared for dogs with or without roaning pattern. Despite the limited power of ΔLRR by having only one marker within the duplication, dogs with one or two copies of the duplication-associated haplotypes (e.g., heterozygotes or homozygotes) showed distinct distributions of ΔLRR, supporting the linkage between the duplication and its flanking markers (FIG. 11). The three roaned dogs without the duplication-associated haplotypes had atypical roaning patterns by having more distinct spots and/or long coats, which were difficult to be accurately called as roaned. One dog with one copy of the duplication-associated haplotype without apparent roaning had ΔLRR=0.15, suggesting that this particular dog likely had the duplication. FIG. 11 shows a density distribution of ΔLRR for the discovery panel dogs with zero, one, or two copies of the duplication-associated haplotypes (no haplotype, heterozygote, and homozygote, respectively). Vertical ticks indicate individual ΔLRR of dogs with roaning (orange) and without roaning (grey). Density plots with the number of individuals less than 10 are not shown, but individual ΔLRR is indicated with longer vertical ticks.

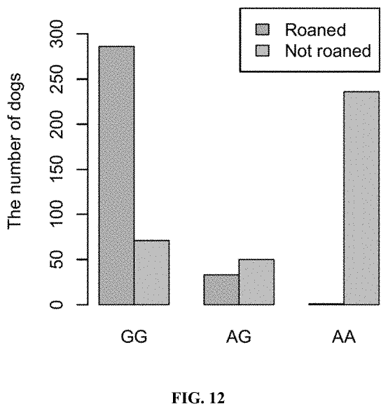

Table 5 shows genotype frequencies of four pigmentation genes in roaned and non-roaned dogs, including A) A-locus: ASIP; B) E-locus: MC1R; C) B-locus: TYRP1; and D) K-locus CBD103. (D) genotype frequencies among blue-eyed (N=147) and brown-eyed (N=47) duplication carriers, indicated as the percent of total (% blue/% brown). FIG. 12 shows Ggenotype frequency of the marker near MITF (CFA20:21836232) in roaned and non-roaned dogs. Using GWAS, a marker near MITF (CFA20:21,836,232) was identified as a region potentially associated with roaning. Roaned dogs were mostly “GG” homozygous (89%) or “AG” heterozygous (10%) at this marker, while “AA” homozygotes were most common in non-roaned dogs (66%), affirming the requirement of a capability of having white areas for roaning to be visible (FIG. 12). Four pigmentation genes that were characterized (A-locus: ASIP, B-locus: TYRP1, E-locus: MC1R, and K-locus: CBD103) did not appear to be associated with roaning (Table 5).

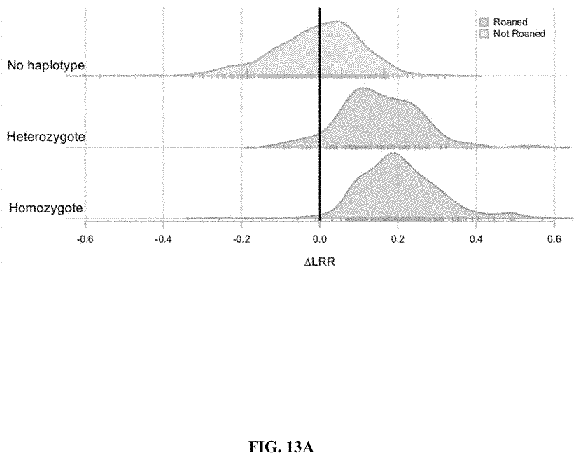

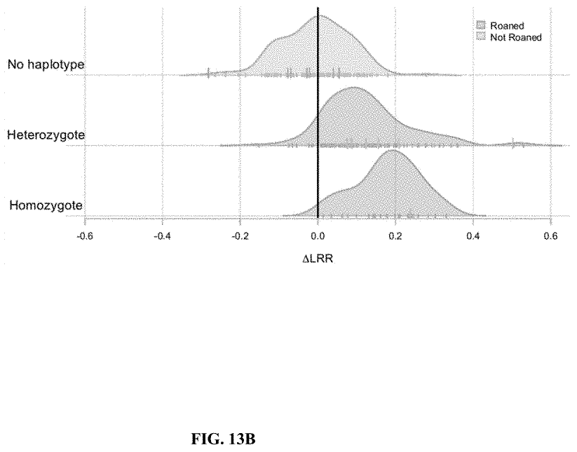

Further, prediction of roaning coat pattern in an 888-dog validation panel was performed as follows. Table 6 shows the number of dogs and breeds in the validation panel. Table 7 shows genotype frequencies of the CFA38 duplication associated with roaning in the validation panel, including A) Target breeds and B) Mixed breeds. FIGS. 13A-13B show a density distribution of ΔLRR for the validation panel dogs with zero, one, or two copies of the duplication-associated haplotypes (no haplotype, heterozygote, and homozygote, respectively), including target breeds (FIG. 13A) and mixed breeds (FIG. 13B). Vertical ticks indicate individual ΔLRR of dogs with roaning (orange) and without roaning (grey). Density plots with the number of individuals less than 10 are not shown, but individual ΔLRR is indicated with longer vertical ticks.

An independent dataset was selected of 586 dogs of 20 target breeds (72 working group dogs in 17 breeds and 514 herding group dogs in 3 breeds) and 302 mixed breed dogs (Table 6) to validate the association between the duplication on CFA38 and roaning. In the target breeds, 95% of dogs with roaning had at least one copy of the duplication-associated haplotypes, while none of the dogs without roaning had the duplication-associated haplotypes (Table 7). Similarly, the duplication was prevalent in the mixed breed dogs (96%), but the duplication heterozygotes were more common than homozygotes due to their mixed ancestry (Table 7). Observations included one duplication homozygous dog and seven heterozygous mixed breed dogs without typical roaned fur. ΔLRR of these eight dogs were all positive (0.08-0.50) (FIGS. 13A-13B), which is indicative of the existence of the duplication in these dogs' genomes. These eight dogs had either small spots (or ticking), faint roaning pattern in muzzle areas, striped coat (e.g., brindle), or wolf-like sable pattern without large patches of roaning.

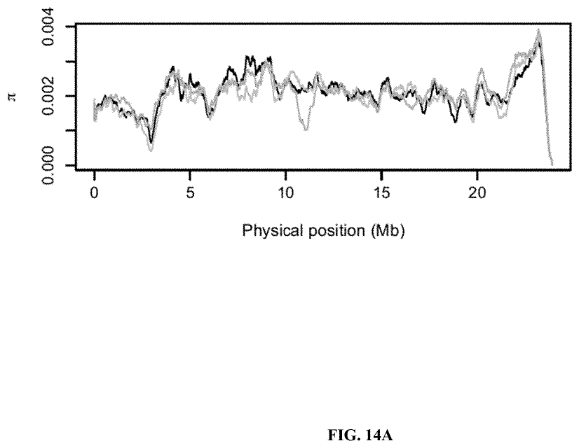

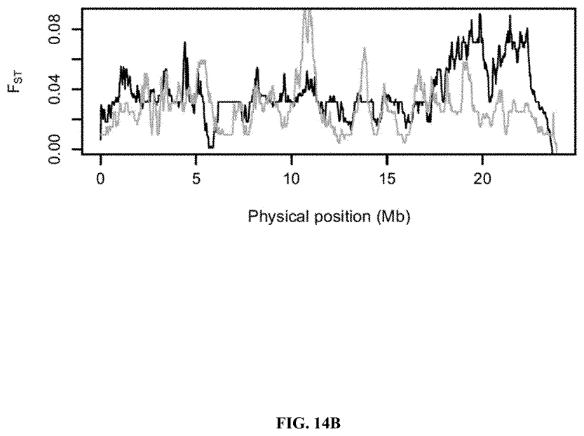

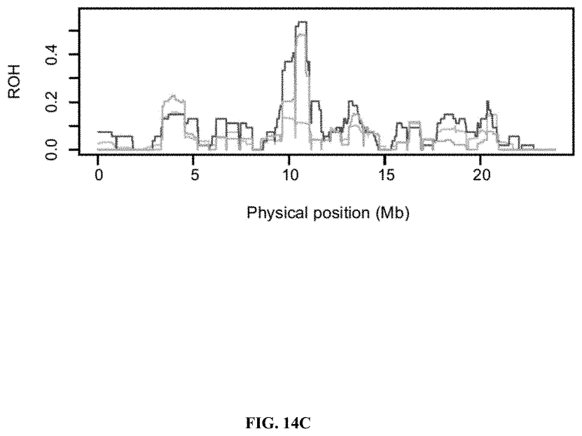

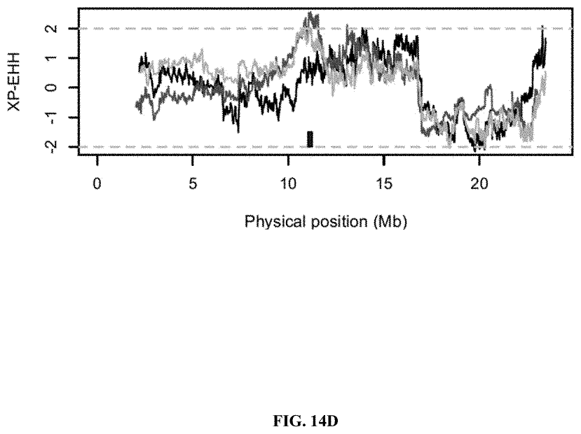

FIGS. 14A-14D show a signature of selection in the region on CFA37 associated with roaning. FIG. 14A shows nucleotide diversity (π) for Wirehaired Pointing Griffon (orange), Border Collies (grey squares), and Labrador Retriever (black triangles) in 500-kb sliding windows. FIG. 14B shows pairwise genetic differentiation (F_{ST}) for Wirehaired Pointing Griffon (red) and Labrador Retriever (black). Border Collies were used as a reference. FIG. 14C shows ROH in Australian Cattle Dog (orange), Dalmatians (red), and Border Collies (grey). FIG. 14D shows XP-EHH in Australian Cattle Dog (orange), Dalmatians (red), and Labrador Retrievers (black). Border Collies were used as a reference. Wirehaired Pointing Griffons and Australian Cattle Dogs are commonly associated with roaning. Blue rectangle: position of the 11-kb duplication. π and F_{ST} are estimated by using whole-genome resequencing data, while ROH and XP-EHH were estimated by using Illumina genotyping data.

Further, selection on the CFA38 was performed as follows. Whole genome re-sequencing data showed that genetic diversity (π) was reduced near the duplication on CFA38 (10-12 Mb) in breeds with high prevalence of roaning, whereas such reduction was not observed in two reference breeds, Border Collies and Labrador Retrievers (FIG. 14A). Genetic differentiation, measured as F_{ST}, was elevated in this region in the comparison between potentially roaned dogs and Border Collies, but not between Labrador Retrievers and Border Collies, which indicates that selection is acting on the duplication haplotype (FIG. 14B). Consistent with the high frequency of the duplication-associated haplotype in this region, the genotype data of the SNP array revealed that about 50% of Australian Cattle Dogs with roaned coat were autozygous between 10 and 11 Mb on CFA38 (FIG. 14C). Similarly, frequent autozygosity was found in Dalmatians but not in Border Collies in this region, indicating that the duplication-associated haplotype was favored by selection in Australian Cattle Dogs and Dalmatians. Moreover, cross-population extended haplotype homozygosity (XP-EI-111) showed significant LD differentiation between Australian Cattle Dogs and Border Collies, where the maximum XP-EHH of 2.05 was found at a marker position 10,871,209 (FIG. 14D). A comparison between Dalmatians and Border Collies also showed high XP-EHH score in this region (2.56 at the position 11,164,866), consistent with the patterns of selection identified by the other analysis.

Further, functional annotation was performed as follows. The most significant GWAS marker (CFA38:11,085,443) was a missense mutation (G->A) leading to an amino acid change from proline to serine of USH2A protein (FIG. 7). However, this may not be the likely causal mutation because about 5% of roaned dogs were GG homozygotes (Table 4). Since the CFA38 duplication was more strongly associated with the roaning, the duplication itself or neighboring mutations were hypothesized to be the causal variant. Variant Effect Predictor (VEP) was performed by using the variants identified in the whole genome re-sequencing of 722 dogs. Results showed that a loss-of-stop-codon mutation was detected at CFA38:11,111,286 (T->C), but all of the putatively roan-associated dogs were homozygous for the wild-type allele at this marker.

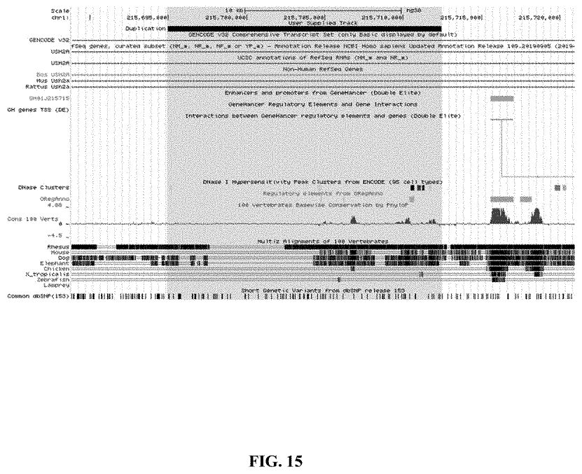

FIG. 15 shows human orthologous region (hg38) of the CFA38 associated with roaning (UCSC genome browser). The highlighted area in blue is the orthologous region to the tandem duplication identified in dogs with roaning, which is located within the intron 61 of USH2A. GeneHancer Regulatory Elements are located at chr1:215,715,579-215,717,032 (green line), which corresponds to CFA38:11,146,170-11,147,605 in the dog genome. DNAse I hypersensitive sites: grey and black boxes. Open Regulatory Annotation (ORegAnno): orange and blue boxes. The CFA38 duplication was detected in an intronic region of USH2A, and the orthologous region in the human reference genome (hg38) was detected at chr1:215,694,945-215,712,452 based on Liftover. At least three clusters of highly conserved sequences were identified in this region (maximum PhyloP scores of 5.5, 4.3, and 4.1), which overlapped with a DNAse I hypersensitive sites and transcription factor binding sites annotated by Open Regulatory Annotation (ORegAnno) (FIG. 15). In addition, there were two additional regions of high conservation outside the duplication (maximum PhyloP scores of 9.6 and 9.1), which were annotated as transcription factor binding sites by ORegAnno and interaction regions by GeneHancer based on Hi-C mapping.

The results showed a novel locus associated with roaning but not with ticking. It may be debated whether T-locus for ticking and R-locus for roaning have similar molecular basis. The results showed that the duplication at 11.13-11.14 Mb on CFA38 was strongly associated with roaning, which indicates that these two theoretical loci are likely located in different genomic regions. Since the custom SNP array has only one marker within the duplication, direct genotyping of the duplication based only on LRR array signal intensity may be challenging. However, owing to the extended linkage in the surrounding region of the duplication, four haplotypes with the duplication were identified. The presence of the duplication in these haplotypes was confirmed by the breakpoint PCR assay, Sanger sequencing of the PCR amplicon spanning the duplication midpoint, and whole-genome re-sequencing data for the identification of discordant read pairs and abrupt read depth increase. In addition, the distribution of LRR signal intensity in dogs with 0, 1, or 2 copies of the duplication-associated haplotypes was in agreement with the expected distribution. This mutation is nearly completely penetrant by explaining more than 95% of roaning cases in both purebred and mixed breed dogs. Thus, the haplotype-based linkage test was performed to accurately detect the presence of the CFA38 duplication, which has a high predictability for roaning coat pattern.

Roaning was observed in dogs with one or two copies of the duplication-associated haplotypes, indicating that the CFA38 duplication is dominant in its effect on fur pigmentation. Although the SNP genotyping array platform did not unambiguously distinguish allelic variants of S-locus, the significant difference at the marker near MITF (FIG. 12) indicates a requirement of certain S-locus variants for the formation of unpigmented fur. A total of nine non-roaned dogs were observed with one or two copies of the duplication in the discovery and validation panels. Assuming that R-locus (e.g., the CFA38 duplication) is a modifier of S-locus (e.g., MITF on CFA20), certain S-locus variants may override the effect of the CFA38 duplication.

The CFA38 duplication was detected in an intronic region of USH2A, and, thus, the exact impact of this duplication on the function of USH2A may be difficult to predict. This is particularly so in dogs where functions of non-coding regions are not well annotated. Nonetheless, the comparative analysis identified putative regulatory regions within and nearby the duplication that are highly conserved in vertebrates (FIG. 15). USH2A encodes a protein, usherin, and mutations in this gene are strongly associated with Usher syndrome in humans, which is characterized by progressive hearing loss and vision impairment often accompanied by retinitis pigmentosa. A functional assay performed by using USH2A knockout mice may show that this gene is involved in the maintenance of retinal photoreceptors and the development of cochlear (inner ear) hair cells. Further, a mutation in USH2A may show abnormal pigment deposition and reduced expression of MITF in retinal cells derived from induced pluripotent stem cells. Further, the distribution of usherin in healthy individuals is highly conserved between mice and humans, in which skin was completely devoid of this protein. The duplication of the putative regulatory regions may result in ectopic expression of USH2A in skin melanocytes. Alternatively, the duplication may facilitate alternative splicing and create a novel protein isoform since this complex gene with 73 exons may form several isoforms.

The evolutionary origin of roaning was investigated. The strong selection signals in the surrounding region of the CFA38 duplication indicate that the frequency of the duplication increased during the formation of modern breeds (FIGS. 14A-14D). In the discovery panel dogs, the duplication is common in Australian Cattle Dogs (94%), Cesky Fousek (100%), English Cocker Spaniel (100%), German Shorthaired Pointer (73%), German Wirehaired Pointer (100%), Spinone Italiano (100%), and Wirehaired Pointing Griffon (100%) (Table 1). In addition, the duplication-associated haplotypes were common in other breeds of pointer and setter groups, such as Weimaraner (90%, N=133) and Vizsla (55%, N=159) (based on a genotype database), but roaned coat, if any, is not visible in these breeds because of the lack of white areas. A sister group of the pointers and setters rarely have the duplication (e.g., 0.1% and 0.8% with sample sizes N=4,591 in Golden Retrievers, and N=2,892 in Labrador Retrievers), and, thus, the duplication likely increased its frequency in the ancestor of the pointer and setter groups by selection acting on roaned coat. For instance, German Shorthaired Pointers with roaned coat may have been favored by hunters because they blend with forest better than white dogs. The duplication-associated haplotypes were sporadically identified in various breeds, including Akitas, Siberian Huskies, and village dogs (e.g., indigenous dogs that accompany humans but are not selectively bred), indicating that selection acted on a variation that existed in the ancestral canine population (e.g., “soft sweep”).

Three loci, namely F-locus, S-locus, and T-locus, may be involved in the formation of distinctive spots in Dalmatians. S-locus may be molecularly characterised, and the s′ variant at MITF may be required to have white fur as a base color. Similar to other breeds with ticking, T-locus may be a responsible locus for creating “ticks” or pigmented spots to the white coat but, with a modifier locus on CFA3, causing fewer and larger spots. Because of the high prevalence of the CFA38 duplication in Dalmatians, it may be hypothesized that Dalmatian's spots have similar genetic basis with roaning, while the interaction between CFA38 duplication and a modifier locus on CFA3 localizes the distribution of pigmented fur. While an involvement of uncharacterized T-locus may occur, the shared haplotype on CFA38 (FIG. 8) and a nested phylogenetic position of Dalmatians within commonly roaned breeds (e.g., English Cocker Spaniel and German Wirehaired Pointer) indicates that the Dalmatian's spots have been derived from roaned ancestors.

The Australian Cattle Dog, as its name suggests, may have been established in Australia in the 19th century by crossing Collie-type dogs with Dingos (a wild dog in Australia), Bull Terriers, Kelpies and Dalmatians. Therefore, the duplication-associated haplotypes on CFA38 may have been introgressed from Dalmatians to the ancestral population of Australian Cattle Dog during its breed formation, followed by selection that increased the frequency of the duplication. This is in line with the above hypothesis because decoupling the allelic combinations at the modifier locus on CFA3 and the roaning locus on CFA38 revealed the putatively ancestral roaning coat pattern.

The results above showed a potential involvement of USH2A in coat pigmentation in dogs by using genome-wide SNP array genotyping and customer-provided photographs. An 11-kb duplication in the intronic region of this gene was strongly associated with roaned coat in both purebred and mixed breed dogs. Since there was no association signal near KITLG on CFA15, KITLG was confirmed to not be responsible for roaning in dogs. These results provide an example of phenotypic convergence where roaned coat has evolved independently in dogs, horses, cattle, among others by using different genes. The similar genetic makeup of the Dalmatian's spots and roaning in other breeds affirms the important role of epistatic interaction in the evolution of novel phenotype. In addition, a tentative causal mutation lies in a non-coding region which may modify expression patterns of USH2A. Darwin may not have been convinced that all of the domestic dog breeds have descended from any one wild species, but novel epistatic interactions and rewiring regulatory networks can result in a burst of phenotypic divergence.

Materials and methods were used as follows, including data collection, genotyping and genome-wide association, identification of tandem duplication, and detecting signatures of selection.

Data collection was performed as follows. Owner-submitted photographs were used to evaluate coat pattern phenotypes of dogs in the database where the owner agreed to participate in scientific research. To ensure a high level of confidence in correctly assessing the phenotype of dogs, specific selection criteria were applied based on the photograph and on the dog itself to determine if each individual was a good candidate for this study. Photographs had to be of relatively high quality, in focus, well-lit, and not show evidence of filter use or image-editing. In addition, photos that included multiple dogs or that depicted a dog very far from the camera were excluded. A reasonable amount or the entirety of the dog's body had to be shown in the photo, especially areas where white spotting (S-locus) patterns are most common (e.g. face, chest, front legs, and feet). Only dogs with clear evidence of white spotting were included. Dogs with residual white (e.g., a single small patch of white on the chest) and light cream to white dogs that were ‘ee’ at the MC1R locus (e.g., very light colored pheomelanin, difficult to distinguish from white spotting) were excluded from the dataset since dogs with these coat patterns could not be reliably phenotyped. For the herding dogs specifically, merle individuals were generally excluded since merle is rarely seen in combination with ticking or roaning and therefore, would be overrepresented in the ‘control’ group. Finally, photos of newborn or extremely young puppies were not considered, since ticking and roaning patterns develop with age.

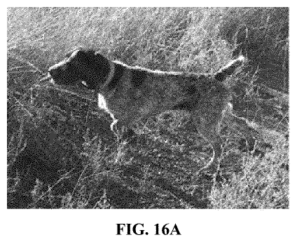

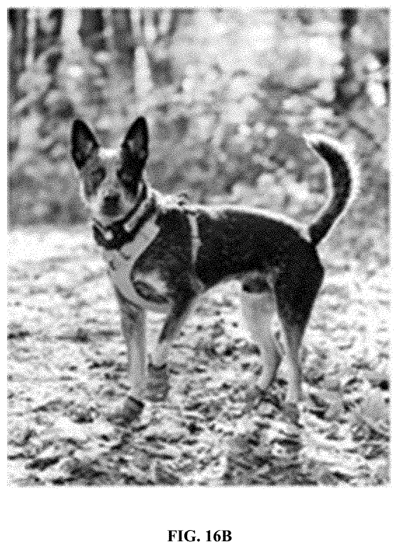

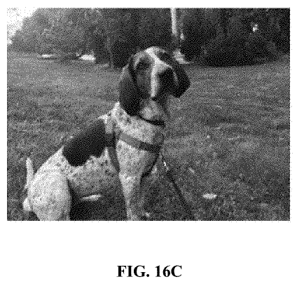

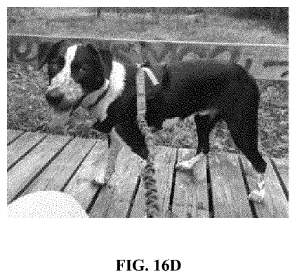

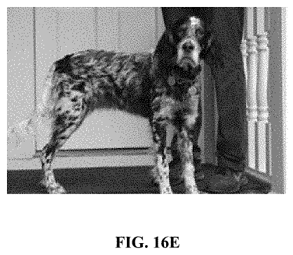

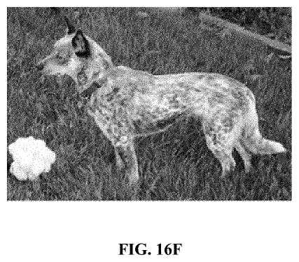

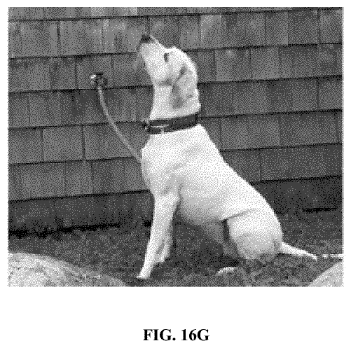

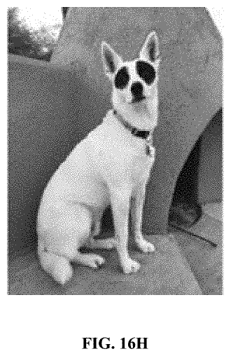

FIGS. 16A-16H show representative coat phenotypes, including German Wirehaired Pointer (roaned) (FIG. 16A); Australian Cattle Dog (roaned) (FIG. 16B); a mixed breed of Treeing Walker Coonhound and Bluetick Coonhound (ticked) (FIG. 16C); a Border Collie (ticked) (FIG. 16D); an English Setter (both roaned and ticked) (FIG. 16E); an Australian Cattle Dog (both roaned and ticked) (FIG. 16F); a Pointer (without roaning and ticking) (FIG. 16G); and an Australian Cattle Dog (without roaning and ticking) (FIG. 16H). FIGS. 16A, 16C, 16E, and 16G are non-herding breeds, while FIGS. 16B, 16D, 16F, and 16H are herding breeds. Dogs were scored for roaning based on their photographs as follows. If roaning was observed on any part of the body, the dog was scored as roaned. Similarly, dogs were classified as ticked if they had any spots on their body, and the extent of ticking was scored from the scale one (lightly ticked) to five (heavily ticked). Because ticking and roaning may result from a similar genetic mechanism, roaned dogs were never considered as ‘not ticked’ controls nor were ticked dogs considered ‘not roaned’ controls, however, dogs may be considered both ticked and roaned if both patterns were clearly visible in the coat. A set of 1,099 adolescent and adult dogs was identified whose coat pattern could be assumed to be developmentally complete (approximately 6 months or older) (FIGS. 16A-16H). A total of 27 breeds were included in the discovery panel (Table 1). First-generation crosses of these breeds or more advanced generation crosses with the proportion of the primary breeds higher than 80% were also included in the discovery panel. Australian Cattle Dogs, Australian Shepherds, and Border Collies were considered as herding group, and the remaining breeds were grouped as working dogs. These dogs were used for the following association studies to identify markers associated with coat color patterns (e.g., discovery panel dogs). Dogs identified as having both ticking and roaning were removed from the discovery panel.

As a separate dataset, coat phenotype data was collected of 529 herding group dogs, 90 working group dogs, and 302 mixed breed dogs to validate the prediction of coat phenotype based on genetic markers (“validation panel”). Mixed breed dogs were selected if the proportion of their primary breeds was less than 50%, and if they were a mix of three or more breeds based on an ancestry prediction algorithm. To represent a mixed breed dog population, a random selection was made of 200 dogs each of the following four groups: 1) dogs with at least one copy of the duplication-associated haplotype on CFA38 and with “AA” genotype at a marker near MITF (the position 11,085,443 on CFA20), 2) dogs with at least one copy of the duplication-associated haplotype on CFA38 and with “GG” genotype at a marker at the marker near MITF, 3) dogs without the duplication-associated haplotype on CFA38 and with “AA” genotype at the marker near MITF, and 4) dogs without the duplication-associated haplotype on CFA38 and with “GG” genotype at the marker near MITF. The MITF marker was included to ensure that the dataset had approximately equal number of dogs with and without white areas in their body. A set of 33 dogs was removed because of the presence of both ticked and roaned areas. To reduce observer bias, all dogs' phenotypes in both discovery and validation panels were scored by the same person, who was blinded to the dog genotypes and their genetic ancestry at the time of phenotyping.

Further, genotyping and genome-wide association were performed as follows. Genotypes of the dogs were collected by using high-density SNP arrays (232,972 markers, of which 228,830 markers were on autosomes and chromosome X). A mean genotyping rate of 97.4% was obtained across all dogs. After removing markers with minor allele frequency less than 1%, a set of 187,496 markers was obtained, for which the genotyping rate was 99.8%. Genotyping rate calculation and marker filtering were performed by PLINK v1.9.

To identify genomic regions associated with coat color variation, a univariate linear mixed model was implemented by GEMMA v0.98.1. To account for confounding effects of shared ancestry among dogs of the same or closely related breeds, a relatedness matrix was constructed from the genotypes of all markers and used as a random effect in the model. Case-control GWAS was performed by using dogs with no ticking and roaning as “controls” and either ticked or roaned dogs as “cases”. The Wald test was used to detect significant association between markers and phenotype by applying a threshold of P<5.0×10⁻⁸.

Further, identification of tandem duplication was performed as follows. LRR (probe intensity) data were retrieved by using Illumina GenomeStudio v2.0.4 (Illumina Inc., San Diego) to identify regions with putative non-balanced SVs near the markers associated with coat pigmentation patterns. To identify SVs in these regions, Manta was used, which uses paired and split-read evidence for SV detection in mapped sequencing reads. To generate mapped sequence reads, whole genome sequence data was obtained for 38 dogs of the eight breeds from the NCBI Sequence Read Archive (Table 2). They were selected because of the high prevalence of ticking and roaning patterns in these breeds. Sequence reads of these samples were mapped to CanFam3.1 reference genome by using the BWA-MEM algorithm in BWA. Read depths for all sites were calculated by using the GATK DepthOfCoverage tool. To visualize the CFA38 duplication breakpoints, mean per site read depths were calculated for non-overlapping 5-kb windows along CFA38 and then were divided by the autosome average read depth for normalization. Discordant read pairs were visualized by Integrative Genomics Viewer (IGV). To identify haplotypes associated with the CFA38 duplication, single nucleotide variants of 722 dogs and other canid species were phased by Beagle v4.1 with default parameter settings. Genetic map positions were derived from a LD-based canine recombination map. Haplotypes of dogs genotyped by a custom microarray were reconstructed by using a reference panel, with missing data imputed using Eagle2.

Haplotypes associated with the CFA38 duplication were validated by a breakpoint PCR assay. Three pairs of primers were designed to amplify three regions: 1) the midpoint spanning the duplication (midpoint primer pair), 2) 5′ flanking region of the duplication start region (5′ control primer pair), and 3) 3′ flanking region of the duplication end region (3′ control primer pair) (Table 2). One microliter of total DNA was used for PCR reactions using the following primer combinations: Tick38-F2-2 and Tick38 R1 (midpoint primer pair), Tick38_F1 and Tick38_R1 (5′ control primer pair), and Tick38-F2-2 and Tick38-R2-2 (3′ control primer pair). All PCR reactions were performed using Go Taq G2 Hot Start Green Master Mix (Promega M7422) in a total volume of 25 microliters (uL) following the manufacturer's protocol. The following cycling parameters were used: 95° C. for 3 minutes, 35× (95° C. for 30 seconds, 58° C. for 30 seconds, and 72° C. for 30 seconds), 72° C. for 5 minutes, and a 12° C. hold. PCR product was visualized on a 1.5% agarose gel with 1× GelRed (Biotium Cat No 41003); product from one dog was submitted for purification and Sanger sequencing at Genewiz (Genewiz.com).

Further, signatures of selection were detected as follows. Pairwise nucleotide diversity (π) was calculated using VCFTools v0.1.16 for Wirehaired Pointing Griffons, Border Collies, and Labrador Retrievers, separately in 500-kb sliding windows with 10-kb steps along CFA38. Genetic differentiation was measured as F_{ST} between breeds (Wirehaired Pointing Griffon vs. Border Collies and Labrador Retrievers vs. Border Collies) in the same window sizes. Whole-genome variant data were used. Sites with missing genotype rate larger than 50% were excluded. Next, array genotype data of the discovery panel was used to calculate runs of homozygosity (ROH) and cross-population extended haplotype homozygosity (XP-EHH). ROH was calculated for Australian Cattle Dogs, Dalmatians, and Border Collies by using PLINK v1.9 in Australian Cattle Dogs, Dalmatians, Border Collies, and Labrador Retrievers with the following parameters (following Sams):
homozyg-window-het 0
homozyg-snp 41
homozyg-window-snp 41
homozyg-window-missing 0
homozyg-window-threshold 0.05
homozyg-kb 500
homozyg-density 5000 (set high to ignore)
homozyg-gap 1000 (set high to ignore)

The frequency of ROH at each marker position was calculated by dividing the sum of ROH state (absence or presence as 0 or 1, respectively) by the total number of individuals. This indicated the proportion of autozygous individuals at a given marker position along a chromosome. XP-EHH was calculated for Australian Cattle Dogs, Dalmatians, and Labrador Retrievers (with Border Collies as a reference breed) by using rehh R package.

### Example 2— Statistical Models for Prediction of Pigmentation Phenotypes in the Domestic Doe from Genetic Markers

In domestic dogs, the intensity of red/fawn color in the hair coat varies from cream (low intensity) to dark red (high intensity) based on the amount of pheomelanin in the hair cells. Three genetic loci may be shown to be significantly associated with this variation in certain breeds, but none of them may be highly predictive of coat color in two of the most popular breeds in the United States, Labrador and Golden Retrievers, or in mixed breed dogs, which comprise the majority of the global canine population. Using a custom Illumina CanineHD Beadchip, a genome-wide association study (GWAS) was performed of 601 purebred Yellow Labrador Retrievers and Golden Retrievers with known coat colors. Three genomic loci were identified that showed significant associations with coat color intensity: canFam3.1 chromosome (chr) 2: 74.7 Mb, chr20: 55.8 Mb, and chr21: 10.9 Mb. The chr2 and chr21 associations may not have been previously reported. The chr2 and chr20 associations were also significant in an independent sample of 630 mixed breed dogs.

GWAS results showed that in most dog breeds, coat color intensity is a polygenic trait, meaning that it is controlled by multiple loci which may interact with each other. In agricultural, livestock, and canine genetics, a common approach for accurately predicting polygenic trait phenotypes is to fit a statistical model with phenotype as a function of genotypes at many markers. For traits that are strongly influenced by genetics (as opposed to environment), a model fit on a sufficiently large and representative “training” sample may be used to accurately predict phenotypes for new individuals given their genotypes, even without knowing the underlying genetic architecture of the trait.

A multiple linear regression model was developed that uses genotype data at 10 genetic markers (SNPs) that were significantly associated with coat color intensity phenotype in the GWAS as the independent variables, and coat color intensity phenotype as the dependent variable. Using this model, a dog's coat color intensity was able to be predicted on a scale of 1 (cream) to 6 (dark red) with at least 70% to 80% accuracy, and whether it has a cream coat versus a darker coat with at least 85% to 95% accuracy (depending on the breed). These findings indicate that direct-to-consumer polygenic tests may be created to predict a dog's coat color intensity phenotype from SNP data, and the expected range of coat color intensity phenotypes in pups produced by the mating of any pair of tested dogs.

Ticking may refer to another type of canine coat color variation, where small pigmented spots of varying numbers and sizes appear on otherwise unpigmented (white) areas of the hair coat. Roaning is similar to and sometimes co-occurs with ticking, but comprises more evenly intermingled pigmented and unpigmented hairs rather than distinct spots. “T locus” and “R locus” may be responsible loci for ticking and roaning, respectively, although they may not have been precisely mapped or characterized at a molecular level. By genotyping 422 dogs with ticking, 386 dogs with roaning, and 357 dogs without ticking or roaning, an 11-kb tandem duplication on chr 38 and a white spotting variant at S locus on chr 29 were identified as likely causal mutations for roaning. Because of the difficulties in directly assaying the duplication by the Beadchip, a linkage test was developed based on six markers in a 73-kb region surrounding the duplication on chr 38 to accurately predict the roaning coat pattern (with 97% accuracy). The linkage test may be used to predict the ticking pattern with at least 55% accuracy, indicating the existence of additional genetic loci associated with ticking.

Tongue color variation is similar to coat color variation in the sense that both show phenotypes of unpigmented, partially pigmented (e.g., spotted or blotched), and completely pigmented patterns. Completely pigmented “black” tongue is a part of the breed standard for some breeds, such as Chow Chows and Shar-Peis. It is possible that dogs with spotted tongues have some proportion of Chow Chow or Shar-Pei ancestry, but the presence of spotted tongues may also occur in purebred dogs of other breeds. Using a total of 4,588 dogs with pink, spotted, or black tongues genotyped on the custom array, a strong association was identified between a 149-kb tandem duplication on chr 37 and tongue pigmentation. Interaction of this duplication with the known pigmentation genes, such as MITF (S locus) and KITLG, may explain the majority of the variation in tongue pigmentation phenotypes. To this end, a multinomial logistic regression model was developed that uses genotype data at these loci to predict tongue pigmentation. This model may be used to predict a dog with a partially or completely pigmented tongue with at least 82% accuracy in both purebred and mixed breed dogs.

The SNP-based test may include numerous advantageous features, including 1) the use of a polygenic prediction models for pigmentation phenotypes, 2) a training panel of a large number of purebred and mixed breed dogs, 3) the use of a set of novel, high-effect genomic loci to predict pigmentation phenotypes, 4) the known accuracy of pigmentation phenotype prediction in a number of breeds as well as mixed breed dogs, and 5) prediction of the expected range of pigmentation phenotypes in litters produced by pairs of tested dogs. For example, while stand-alone genetic tests may be performed for single mutations (e.g., one chr20 mutation) associated with coat color intensity in several breeds, polygenic tests may be developed to predict coat color intensity phenotype in Labrador and Golden retrievers or mixed breed dogs, and/or to use information from more than one genetic locus, and/or to cover the additional pigmentation phenotypes of ticking and/or roaning and tongue pigmentation. As part of a direct-to-consumer genetic test, a dog's predicted pigmentation phenotypes may be used in conjunction with a matchmaker tool to plan matings between pairs of dogs that are more likely to produce the desired phenotype while minimizing genome-wide inbreeding levels and risk for over 180 genetic health conditions.

Coat color intensity phenotype prediction was performed as follows. A coat color intensity phenotype prediction model was constructed that uses genotype data at 10 SNP markers on chr2 (n=5), chr20 (n=1), chr21 (n=3), and chr15 (n=1) that are currently included on a custom Illumina CanineHD Beadchip. These are shown in Table 8.

TABLE 8
Markers used in the model for predicting color coat intensity

| | | | Associated | Other |
|---|---|---|---|---|
| Chr | SNP ID | Position | allele | allele |
| 2 | BICF2P1302896 | 74746906 | A | T |
| 2 | TIGRP2P30892_rs8643466 | 75066994 | A | G |
| 2 | BICF2P202986 | 75412412 | G | A |
| 2 | TIGRP2P31085_rs8981024 | 75658840 | A | G |
| 2 | BICF2S245539 | 77731034 | A | C |
| 15 | BICF2G630433130[1] | 29840789 | G | A |
| 20 | BICF2P828524[2] | 55850145 | G | A |
| 21 | BICF2G630655699 | 10898947 | G | A |
| 21 | BICF2S23541470 | 11080478 | A | C |
| 21 | BICF2P1392970 | 12216058 | A | G |

This set of markers may be used to accurately predict coat color intensity phenotype (e.g., by applying the following equation).

Ŷ=−0.77+1.33×₁+0.92×₂+0.17×₃+0.23×₄+0.31×₅+0.05×₆+0.71×₇+0.10×₈+0.12×₉+0.15×₁₀

where Ŷ is the predicted numeric phenotype value and X₁ through X₁₀ are the number of alleles associated with darker coat color that the dog of interest has at BICF2P1302896, BICF2P828524, BICF2G630655699, BICF2G630433130, TIGRP2P30892 rs8643466, TIGRP2P31085 rs8981024, BICF2S245539, BICF2P1392970, BICF2P202986, and BICF2S23541470 (respectively).

If the Ŷ value for the dog is less than or equal to 1.5, the dog is classified as likely to have a cream coat; and if Ŷ is greater than 1.5, the dog is classified as likely to have a yellow or red coat. For any pair of dogs with SNP genotypes at these 10 markers, all possible genotype combinations can be determined in pups produced by mating those dogs. By processing these data using the above equation, the predicted coat color intensity phenotype may be obtained for each genotype combination. As a result, the predicted range of coat color intensity phenotypes, as well as the expected frequency of each phenotype, may be reported in litters produced by that pair of parents.

TABLE 9
Coat color intensity phenotype predictive model accuracy

Breed

| Model | Goldens | Labs | Mixed | All |
|---|---|---|---|---|
| Pred. vs. Obs. 6 pt | 0.745 | 0.8 | 0.702 | 0.819 |
| Quant Phen R-squared | | | | |
| Binned Cream vs. Not | 0.971 | 0.945 | 0.877 | 0.89 |
| Cream Accuracy | | | | |

TABLE 10
Red-associated allele frequencies by breed in GWAS samples for coat color intensity predictive model SNPs

| | | Breed | | | | | | | | |
|---|---|---|---|---|---|---|---|---|---|---|
| | | | | American | | | | | | |
| | | | Bichon | Eskimo | Golden | Labrador | Mixed | Irish | | Duck |
| | | Samoyed | Frise | Dog | Retriever | Retriever | Breed | Setter | Vizsla | Toller |
| | | Phenotype (6 pt scale) | | | | | | | | |
| | | 1 | 1 | 1 | 1 to 6 | 1 to 6 | 1 to 6 | 5 to 6 | 5 to 6 | 5 to 6 |
| SNPs | BICF2P1302896 | 0 | 0.16 | 0.02 | 0.29 | 0.22 | 0.48 | 1 | 1 | 0.98 |
| | TIGRP2P30892_rs8643466 | 0.06 | 0.01 | 0.09 | 0.21 | 0.09 | 0.15 | 0.11 | 0.04 | 0.04 |
| | BICF2P202986 | 0.9 | 0.88 | 0.83 | 0.53 | 0.91 | 0.86 | 1 | 0.92 | 0.92 |
| | TIGRP2P31085_rs8981024 | 0.12 | 0.23 | 0.76 | 0.28 | 0.38 | 0.43 | 0.52 | 0.48 | 0.15 |
| | BICF2S245539 | 0.98 | 0.78 | 0.96 | 1 | 1 | 0.96 | 1 | 1 | 1 |
| | BICF2G630433130 | 0.24 | 0.63 | 0.04 | 0.79 | 0.8 | 0.53 | 1 | 1 | 0.56 |
| | BICF2P828524 | 0.08 | 0.15 | 0.12 | 0.77 | 0.68 | 0.64 | 1 | 1 | 1 |
| | BICF2G630655699 | 0.69 | 0.28 | 0.32 | 0.41 | 0.2 | 0.39 | 0.57 | 0.38 | 0.02 |
| | BICF2S23541470 | 0.14 | 0.61 | 0.58 | 0.57 | 0.32 | 0.45 | 0.17 | 0.38 | 0.25 |
| | BICF2P1392970 | 0.48 | 0.45 | 0.44 | 0.59 | 0.5 | 0.53 | 0.3 | 0.1 | 0.5 |

Interactions between coat color intensity and other coat color phenotypes were tested, including: the E Locus (MC1R), which determines whether both eumelanin (black pigment) and pheomelanin (red/yellow pigment), or just pheomelanin may be expressed in hair; the K locus (TYRP1), which determines whether or not A locus genotype is expressed; and the A locus (ASIP), which determines pattern of black and red in coat (e.g., sable, tan points).

For example, if a dog is “ee” at E locus, then if the dog is not “aa” at A locus, then “Red” areas of the dog will have reported intensity, else the dog is solid black/brown. If the dog is not “ee” at E locus, then if the dog is “kyky” at K locus and is not “aa” at A locus, then “Red” areas of the dog will have reported intensity. Else, if the dog is not “ee” at E locus and is not “kyky” at K locus, then the dog is solid black/brown.

Further, roaned coat pattern was predicted as follows. A roaning coat pattern prediction model was constructed that uses genotype data at seven SNP markers on chr20 (n=1) and chr38 (n=6) that are currently included on a custom Illumina CanineHD Beadchip. These are shown in Table 11.

TABLE 11
Markers used in the model for predicting roaned coat pattern

| | | | Associated | Other |
|---|---|---|---|---|
| Chr | SNP ID | Position | allele | allele |
| 20 | EMB_chr20_21836232 | 21836232 | G | A |
| 38 | chr38_11085443 | 11085443 | A | G |
| 38 | BICF2P941536 | 11106067 | G | A |
| 38 | chr38_11111286 | 11111286 | A | G |
| 38 | BICF2S23536290 | 11120096 | G | A |
| 38 | BICF2P1396284 | 11154832 | A | C |
| 38 | BICF2S23332370 | 11158400 | A | G |

The marker on chr20 is in the putative regulatory region of MITF (S locus). For roaned (pigmented) hairs to be visible, two copies of the “G” variant are required to make the base coat color white, since this variant is recessive. The duplication on chr38 is located between BICF2S23536290 and BICF2P1396284. The following allelic combinations of the six markers on chr38 were strongly associated with the duplication: AGAGAA, GGAGAA, GAAAAA, and GGAAAA. Since the duplication on chr38 is a dominant modifier of the white coat pattern by MITF (GG homozygotes), the roaning coat pattern is predicted if a dog has: 1) at least one copy of the duplication-associated allelic combinations (AGAGAA, GGAGAA, GAAAAA, and GGAAAA) at R locus on chr 38; and 2) two copies of the G variant at S locus on chr 20. Table 12 shows a model prediction accuracy for roaned coat pattern.

TABLE 12
Model prediction accuracy for roaned coat pattern

| | Breed | | |
|---|---|---|---|
| Model | Non-herding | Herding | All |

| | | |
|---|---|---|
| 0.903 | 0.993 | 0.967 |

Non-herding breeds used included: English Setter, Llewellin Setter, Pointer, Clumber Spaniel, Irish Setter, Springer Spaniel, Brittany, Cesky Fousek, Wirehaired Pointing Griffon, Spinone Italiano, German Shorthaired Pointer, German Longhaired Pointer, German Wirehaired Pointer, Cocker Spaniel, Pudelpointer, Bluetick Coonhound, American English Coonhound, English Foxhound, Harrier, American Foxhound, Hamiltonstovare, Plott, Treeing Walker Coonhound, Munsterlander, and Stabyhoun. Herding breeds used included: Border Collies, Australian Shepherds, and Australian Cattle Dogs.

Further, tongue pigmentation phenotype was predicted as follows. A tongue pigmentation prediction model was constructed that uses direct genotyping of the 149-kb tandem duplication based on 21 markers (canFam3.1 position chr37: 28,543,289-28,692,507) or genotyping of linked markers used to predict duplication genotype (e.g. the [G/A] SNP at position chr37:28,616,075), as well as direct genotyping of TMEM40 or linked markers (e.g., the [A/G] SNP at position chr20:5,843,762), MITF or linked markers (e.g. the [A/G] SNP at position chr20:21,836,232) and direct genotyping of KITLG or linked markers (e.g., chr15:29,840,789). The copy number (CN) of the duplicated region on chr 37 was determined based on the signal intensity of the probes on a custom Illumina CanineHD Beadchip as well as the estimated number of risk alleles. Table 13 shows the markers used in the model for predicting tongue pigmentation phenotype.

TABLE 13
Markers used in the model for predicting tongue pigmentation

| | | | Associated | Other |
|---|---|---|---|---|
| Chr | SNP ID | Position | allele | allele |
| 15 | BICF2G630433130 | 29840789 | G | A |
| 20 | EMB_chr20_5843762 | 5843762 | A | G |
| 20 | EMB_chr20_21836232 | 21836232 | A | G |
| 37 | BICF2G630133910 | 28544660 | A | G |
| 37 | chr37_28548746 | 28548746 | A | G |
| 37 | BICF2G630133930 | 28553736 | G | A |
| 37 | BICF2P888958 | 28559662 | G | A |
| 37 | BICF2S2332765 | 28565454 | A | G |
| 37 | BICF2S2332764 | 28565737 | G | A |
| 37 | BICF2G630133940 | 28568693 | C | A |
| 37 | BICF2G630133952 | 28586737 | G | A |
| 37 | BICF2G630133969 | 28592172 | G | A |
| 37 | BICF2G630133994 | 28611801 | A | G |
| 37 | BICF2G630134000 | 28614027 | A | G |
| 37 | BICF2G630134015 | 28615953 | G | C |
| 37 | chr37_28616075 | 28616075 | G | A |
| 37 | BICF2G630134036 | 28625580 | G | A |
| 37 | BICF2P387308 | 28627370 | G | A |
| 37 | chr37_28636710 | 28636710 | A | G |
| 37 | BICF2S23141462 | 28643755 | A | G |
| 37 | BICF2G630134058 | 28655276 | A | G |
| 37 | BICF2G630134067 | 28665036 | C | A |
| 37 | BICF2G630134080 | 28679081 | A | G |
| 37 | BICF2G630134084 | 28682925 | G | A |

The presence of the duplication on chr 37 explained 95% of the black tongue cases (N=131/138) and 28% of the spotted tongue cases (N=200/716), whereas only 5% of the pink tongue dogs carried the duplication (N=133/2,705). To predict tongue color phenotypes by taking into account the genotypes of the other three loci on chr 15 and chr 20, a multinomial logistic regression model was constructed. The duplication on chr 37 was observed to be the largest additive effect on the model for predicting both partially and completely pigmented tongues 03=1.995 and 4.185, respectively, Table 14). All four loci significantly contributed to the model for predicting spotted tongue, whereas the marker on chr 15 had no significant additive effect on the prediction of the black tongue phenotype 03=0.421, p=0.07). Table 14 shows effect sizes of four loci associated with tongue pigmentation (partial or complete) by multinomial logistic regression analysis.

TABLE 14
Effect sizes (β) of four loci associated with tongue pigmentation
(partial or complete) by multinomial logistic regression analysis.

| | | β (partial | | β (complete | |
|---|---|---|---|---|---|
| Chr | Gene* | pigmentation) | | pigmentation) | |
| 15 | KITLG | 0.446 | (1 × 10⁻⁸) | 0.421 | (0.07) |
| 20 | TMEM40 | 1.104 | (<1 × 10⁻¹⁶) | 2.665 | (<1 × 10⁻¹⁶) |
| 20 | MITF | 1.095 | (<1 × 10⁻¹⁶) | 1.998 | (9 × 10⁻¹⁰) |
| 37 | duplication | 1.995 | (<1 × 10⁻¹⁶) | 4.185 | (<1 × 10⁻¹⁶) |

Numbers in brackets indicate p-values, and “*” indicates the closest genes to the most significant markers.

### Example 3—R-Locus for Roaned Coat is Associated with a Tandem Duplication in an Intronic Region of USH2A in does and Also Contributes to Dalmatian Spotting

Structural variations (SVs) may represent a large fraction of all genetic diversity, but how this genetic diversity is translated into phenotypic and organismal diversity may be unclear. Explosive diversification of dog coat color and patterns after domestication can provide a unique opportunity to explore this question; however, a significant obstacle is to efficiently collect a sufficient number of individuals with known phenotypes and genotypes of hundreds of thousands of markers. Using customer-provided information about coat color and patterns of dogs tested on a commercial canine genotyping platform, a genomic region on chromosome 38 was identified that is strongly associated with a mottled coat pattern (roaning) by genome-wide association study. A putative causal variant was identified in this region, an 11-kb tandem duplication (11,131,835-11,143,237) characterized by sequence read coverage and discordant reads of whole-genome sequence data, microarray probe intensity data, and a duplication-specific PCR assay. The tandem duplication is in an intronic region of usherin gene (USH2A), which was perfectly associated with roaning but absent in non-roaned dogs. Strong selection signals were detected in this region characterized by reduced nucleotide diversity (π), increased runs of homozygosity, and extended haplotype homozygosity in Wirehaired Pointing Griffons and Australian Cattle Dogs (typically roaned breeds), as well as elevated genetic difference (F_{ST}) between Wirehaired Pointing Griffon (roaned) and Labrador Retriever (non-roaned). Surprisingly, all Dalmatians (N=262) carried the duplication embedded in identical or similar haplotypes with roaned dogs, indicating this region as a shared target of selection during the breed's formation. The results indicate that the Dalmatian breed's unique spots were a derived coat pattern by establishing a novel epistatic interaction between roaning “R-locus” on chromosome 38 and an uncharacterized modifier locus. These results demonstrate the utility of consumer-oriented genotype and phenotype data in the discovery of genomic regions contributing to phenotypic diversity in dogs.

Diverse coat colors and patterns observed in dogs and other domestic animals have long fascinated breeders. Geneticists and evolutionary biologists have taken interest in this remarkable diversity because it provides an excellent model to study 1) how numerous phenotypes can be established from morphologically less diverse wild counterparts, 2) how changes in selection pressure alter genotype-phenotype interactions, and 3) how repeated evolution of similar coat phenotypes in multiple species is attainable. Studies of the genetics of coat color have led to the identification of a number of genes that have been repeatedly involved in the formation of diverse color morphs in both wild and domesticated animals [ref 1]. One of the genes involved in the repeated evolution of light and dark color polymorphisms is the melanocortin-1 receptor (MC1R) that modulates the activity of the melanin synthesis pathways in the fur, plumage, scales and skin of mammals, birds, reptiles, amphibians, and fish [ref 2]. In other cases, similar coloration has independently evolved in multiple lineages via mutations in different genes (e.g., LYST and AIM1 in polar bears and KIT and MATP in horses with white coats) [refs. 3-5]. Understanding the genetic mechanisms of color variation and phenotypic convergence has shed light on how novel phenotypes evolve under similar selective forces (either natural or artificial). In addition, high conservation of melanogenesis pathways across vertebrates warrants transformative research in human genomics research, such as the case of MC1R that is strongly associated with increased risk for melanoma [refs. 6-7].

Ticking and roaning are two common coat patterns observed in dogs and other domestic animals. Ticking may be characterized as small pigmented spots of varying numbers and sizes appearing on otherwise unpigmented (white) areas. Roaning may be similar to and sometimes co-occurs with ticking but may be characterized with pigmented and unpigmented hairs interspersed more evenly without the formation of distinct spots. The distinctive spots of the Dalmatian breed may be believed to be a modified form of ticking where a size of each tick or spot is enlarged and distinctive by a modifier locus (flecking locus, F-locus) mapped on canine chromosome (CFA) 3 [ref 8]. Typically, individuals are born without ticking, Dalmatian's spotting, and roaning patterns, but instead these pigmented areas develop as the individual ages, indicating time-dependent action of underlying pigmentation mechanisms. In dogs, two theoretical loci, namely “T-locus” and “R-locus”, may be proposed as responsible loci for ticking and roaning, respectively, although it may be unclear whether they constitute the same genomic regions with different variants because they have not been characterized at a molecular level [ref 9]. Similarly, the molecular basis of F-locus is unknown. KIT ligand gene (KITLG) may be reported as a causal gene for roaning in cattle [refs. 10-11], pigs [refs. 12-13], and goats [ref 14], but may not be involved in roaning in dogs [ref 9].

Gene interaction or epistasis is a key mechanism in the formation of phenotypic diversity in both wild and domesticated species. An example is three color types of Labrador Retrievers, where tyrosinase-related protein 1 (TYRP1) and MC1R determine their coat colors as black, chocolate, or yellow [ref 15]. Modifier genes constitute a type of epistasis; for example, several variants of microphthalmia-associated transcription factor (MITE) modify the coat color of dogs by preventing the melanocyte development and migration in certain areas of the body and, in some cases, across nearly the entire body. This results in a loss of pigmentation leading to white markings in otherwise uniformly colored areas [refs. 16-18]. S-locus is a major locus controlling this white spotting pattern, and several variants within and close to MITF have been identified, including a SINE insertion at 3 kilobase (kb) upstream of the MITF transcription start site (TSS) and a variable length polymorphism (Lp) at 100 bp upstream of MITF TSS [refs. 17-18]. Both T-locus and R-locus are considered as modifier loci by locally changing coat color from white to pigmented through the interaction with S-locus [ref 9]. Furthermore, unique spots found exclusively in Dalmatians may be a result of an epistatic interaction between T-locus and F-locus on CFA3 in linkage with a hyperuricosuria-associated variant in SLC2A9 [ref 19]; however, it may not be known how SLC2A9 itself or other genes linked to it can modify pigmentation patterns via T-locus.

Genomic regions associated with ticking and roaning coat patterns in dogs were investigated by using a total of 1,281 purebred dogs for marker discovery (“discovery panel”) and 274 mixed breed dogs for marker validation (“validation panel”) that were genotyped using an Embark SNP array with 220,484 markers covering all 38 autosomes and chromosome X. Dog owners contributed to this study by providing photographs of their dogs, from which their phenotypes were classified as ticked, roaned, or lacking these patterns to identify genomic regions associated with these phenotypes by genome-wide association study (GWAS). For the discovery of the associated genomic regions, a diverse set of purebred dogs were selected in 27 breeds that commonly exhibit white spotting patterns caused by genetic variants at the S-locus. Pigmented “case” dogs were defined on the basis of specific criteria in the extent of ticking and/or the presence of roaning patterns, whereas unpigmented “control” dogs were selected from the same breed cohorts with white spotting patterns but without any evidence of ticking and roaning. Both case and control dogs included in the study, thus, exhibited white spotting patterns. Dogs without white spotting patterns, including those that appear white as a result of dilute pheomelanin and those with a residual white phenotype were excluded from the study to ensure the uniformity of the base coat color. In addition, publicly available whole-genome re-sequencing data were used to fine-map the location of a putative causal variant associated with roaning. The genotype-phenotype association was robust, as phenotypes of mixed breed dogs (an independent validation dataset) were accurately predicted by their genotypes. Finally, phylogenetic analysis of the putative causal variant was performed to examine signatures of positive selection and identify whether any other coat patterns might be associated with the variant.

Phenotype data collection was performed as follows. Owner-submitted photographs were used to evaluate coat patterns of dogs in a veterinary database where the owner agreed to participate in scientific research. To ensure a high level of confidence in correctly assessing the coat patterns, the following selection criteria were applied based on the photograph and on the dog itself to determine if each individual was a good candidate for the study. Photographs had to be of high quality, in focus, well-lit, and not show evidence of filter use or image-editing. In addition, photographs that included multiple dogs or that depicted a dog very far from the camera were excluded. A reasonable amount or the entirety of the dog's body had to be shown in the photograph, especially areas where white patterns likely governed by S-locus [refs. 17-18] were common (e.g., face, chest, front legs, and feet). Dogs with the following three types of coat patterns were removed from the dataset to minimize the bias between case and control groups as well as to remove dogs with white areas in their coat by other pigmentation loci: “residual white” (a small patch of white on the chest), merle by M-locus (M/M and M/m) [ref 20], and light cream coat likely under the influence of E-locus (e/e) and I-locus (i/i) [ref 21]. The selection of dogs was based on phenotypes without referring to genotypes of these loci except E-locus, where e/e homozygotes (SNP marker at CFA5:63694334 in MC1R) were excluded from the study. It is common to see a white patch even on the chest of ticked and roan dogs, thus, control dogs especially had to have white areas extending beyond the chest area into the legs and/or face. Dogs without sufficient white spotting patterns may otherwise be mis-phenotyped, since ticking and roan are not visible on a solid pigmented background. Finally, photos of newborn or juvenile dogs were not considered, since ticking and roaning patterns may develop with age.

If roaning was observed on any part of the body, the dog was scored as roaned. Similarly, dogs were classified as ticked if they had any spots on their body, and the extent of ticking was scored either the scale one (lightly ticked) or two (heavily ticked). Because ticking and roaning may result from a similar genetic mechanism, roaned dogs were never considered as ‘not ticked’ controls, nor were ticked dogs considered ‘not roaned’ controls. However, dogs could be considered both ticked and roaned if both patterns were clearly visible in the coat. A set of 1,281 adolescent and adult dogs were identified whose coat pattern may be assumed to be developmentally complete (approximately 6 months or older). A total of 27 breeds were included in the discovery panel. Australian Cattle Dogs, Australian Shepherds, and Border Collies were considered as a herding group, and the remaining 24 breeds were grouped as non-herding dogs. Breeds that never or rarely exhibit white spotting patterns were not included in the discovery panel. These dogs were used for the following association studies to identify markers associated with coat color patterns (e.g., discovery panel dogs). A random subset of 100 dogs were phenotyped by the same individual a second time several weeks later to evaluate phenotyping consistency. Overall concordance was 98% for the roan phenotype (binary) and 98% for the ticking phenotype (no ticking, light ticking, or heavy ticking). All discordant calls were either the changes from the absence of roaning to inconclusive or from heavy to light ticking.

The definition of the F-locus for Flecking is unclear. While the term flecking has been sometimes used synonymously with ticking and/or roaning (e.g., images.akc.org/pdf/judges/groups/Sporting_Group.pdf), other definitions suggest that flecking is unpigmented hairs within pigmented areas (e.g., white hairs within a pigmented spot). One of the distinctions between Dalmatian spotting and ticking may be the presence or absence of white hairs within the spots; Dalmatian spots have completely solid pigment while ticks contain interspersed white hairs [ref 22]. The latter definition was adopted, and F-locus was used as a Dalmatian's spot modifier where a homozygous recessive genotype (f/f) makes completely solid spots.

As a separate dataset, coat phenotype data was collected of mixed breed dogs by applying the same phenotype selection criteria with the discovery panel to validate the prediction of coat phenotype based on genetic markers (“validation panel”). To represent a mixed breed dog population, 400 dogs were initially randomly selected with at least one copy of the duplication-associated haplotypes, and another 400 dogs were selected without it. Then the same photo selection criteria were applied with the discovery panel to choose 274 mixed breed dogs. To reduce observer bias, all dog's phenotypes in both discovery and validation panels were scored by one individual, who was blinded of the dog genotypes and their genetic ancestry at the time of phenotyping.

Genotyping and genome-wide association were performed as follows. DNA was extracted from buccal swab samples collected by dog owners and extracted by Illumina, Inc. Genotypes of the dogs were collected by using custom Illumina Canine high-density SNP arrays (a total of 220,484 markers). Mean genotyping rate was 97.4% across all dogs. After removing markers with minor allele frequency less than 1%, a set of 176,910 markers was used, for which the genotyping rate was 99.8%. Genotyping rate calculation and marker filtering were performed by PLINK v1.9 [ref 23].

To identify genomic regions associated with coat color variation, a univariate linear mixed model was applied and implemented in GEMMA v0.98.1 [ref 24]. To account for confounding effects of shared ancestry among dogs of the same or closely related breeds, a relatedness matrix was constructed from the genotypes of all autosomal markers and used as a random effect in the model. Case-control GWAS was performed by using dogs with no ticking and roaning as “controls” and either ticked or roaned dogs as “cases”. The Wald test was used to detect significant association between markers and phenotype by applying a threshold of P<5.0×10⁻⁸. To delineate a region associated with roaning on CFA38, haplotypes of roaned and non-roaned dogs were reconstructed from the array genotypes by using Beagle v4.1 with default parameter settings [ref 25]. Genetic map positions were derived from a LD-based canine recombination map [ref 26].

Identification of tandem duplication was performed as follows. LRR (probe intensity) data were retrieved of 1,182 dogs (346 roaned and 567 control dogs in the discovery dataset, 118 and 151 roaned and control dogs in the validation dataset) using Illumina GenomeStudio v2.0.4 (Illumina Inc., San Diego) to identify regions with putative non-balanced SVs near the markers associated with coat pigmentation patterns. To identify SVs in these regions, Manta [ref 27] was used, which uses paired and split-read evidence for SV detection in mapped sequencing reads. To generate mapped sequence reads, whole-genome sequence data was obtained for 38 dogs of the eight breeds from the NCBI Sequence Read Archive (www.ncbi.nlm.nih.gov/sra). These eight were selected because of the high prevalence of ticking and roaning patterns in these breeds. Sequence reads of these samples were mapped to CanFam3.1 reference genome by using the BWA-MEM algorithm in BWA [ref 28]. Read depths were calculated for all sites using the GATK DepthOfCoverage tool [ref 29]. To visualize the CFA38 duplication breakpoints, mean per site read depths were calculated for non-overlapping 5-kb windows along CFA38 and then were divided by the autosome average read depth for normalization. Discordant read pairs were visualized with Integrative Genomics Viewer (IGV) [ref 30]. To identify haplotypes associated with the CFA38 duplication, genotypes were phased of bi-allelic variants of 722 dogs and other canid species [ref 31] with Beagle v4.1 with default parameter settings [ref 25].

Haplotypes associated with the CFA38 duplication were validated by a breakpoint PCR assay. Three pairs of primers were designed to amplify three regions in separate PCR reactions: 1) the midpoint spanning the duplication (midpoint primer pair), 2) 5′ flanking region of the duplication start region (5′ control primer pair), and 3) 3′ flanking region of the duplication end region (3′ control primer pair). One microliter of total DNA was used for PCR reactions using the following primer combinations: Tick38-F2-2 and Tick38_R1 (midpoint primer pair), Tick38_F1 and Tick38_R1 (5′ control primer pair), and Tick38-F2-2 and Tick38-R2-2 (3′ control primer pair). All PCR reactions were performed using Go Taq G2 Hot Start Green Master Mix (Promega M7422) in a total volume of 25 uL following the manufacturer's protocol. The following cycling parameters were used: 95° C. for 3 minutes, 35× (95° C. for 30 seconds, 58° C. for 30 seconds, 72° C. for 30 seconds), 72° C. for 5 minutes, 12° C. hold. PCR product was visualized on a 1.5% agarose gel with 1× GelRed (Biotium Cat No 41003); the products from three dogs were submitted for purification and Sanger sequencing at Biotechnology Resource Center at Cornell University.

Detecting signatures of selection was performed as follows. Pairwise nucleotide diversity (π) was calculated using VCFTools v0.1.16 [ref 32] for Wirehaired Pointing Griffons, Border Collies, and Labrador Retrievers, separately in 500-kb sliding windows with 10-kb steps along CFA38. Genetic differentiation was measured as F_{ST} between breeds (Wirehaired Pointing Griffon vs. Border Collies and Labrador Retrievers vs. Border Collies) in the same window sizes. Whole-genome variant data reported in [ref 31] were used. Sites with missing genotype rates larger than 50% were excluded. Next, an array genotype data of the discovery panel was used to calculate runs of homozygosity (ROH) and cross-population extended haplotype homozygosity (XP-EHH) [refs. 33-34]. ROH was calculated for Australian Cattle Dogs, Dalmatians, and Border Collies by using PLINK v1.9 [ref 23] in Australian Cattle Dogs, Dalmatians, Border Collies, and Labrador Retrievers with the following parameters (following [ref. 35]):
homozyg-window-het 0
homozyg-snp 41
homozyg-window-snp 41
homozyg-window-missing 0
homozyg-window-threshold 0.05
homozyg-kb 500
homozyg-density 5000 (set high to ignore)
homozyg-gap 1000 (set high to ignore)

The frequency of ROH at each marker position was calculated by dividing the sum of ROH state (absence or presence as 0 or 1, respectively) by the total number of individuals. This indicated the proportion of autozygous individuals at a given marker position along a chromosome. XP-EHH was calculated for Australian Cattle Dogs, Dalmatians, and Labrador Retrievers (with Border Collies as a reference breed) by using rehh R package [ref 34].

Participating dogs were part of a veterinary customer base. Owners provided informed consent to use their dogs' data in scientific research by agreeing the following statement: “I want this dog's data to contribute to medical and scientific research”. Ethical approval was not required as non-invasive methods for genotype or phenotype collection were used (buccal swab and photographing, respectively). Dogs were never handled directly by researchers. Owners were given the opportunity to opt-out of the study at any time during data collection.

A novel association on chromosome 38 was observed with roaning, but not with ticking. A total of 1,281 purebred dogs was selected with profile pictures where dogs showed white spotting patterns in their bodies. Inspection of customer-provided photographs identified 344 dogs with varying degrees of ticking, 358 dogs with a roaning pattern on some part of the body, and 579 dogs without any noticeable ticking or roaning in any part of their bodies (e.g., “control” dogs). Dogs that exhibited both phenotypes (ticking and roaning) were excluded from the study. The extent of ticking was further classified as either lightly ticked (n=168) or heavily ticked (n=176), and these ticking scores were treated as an ordinal variable in the subsequent GWAS (together with the control dogs). Since dogs with residual white and dilute pheomelanin were excluded from the study (see Methods), it was assumed that all dogs selected for the study with white areas had one or two copies of the following S-locus alleles: extreme white (s^{w}), piebald (s^{p}), and irish white (sⁱ) [refs. 17-18].

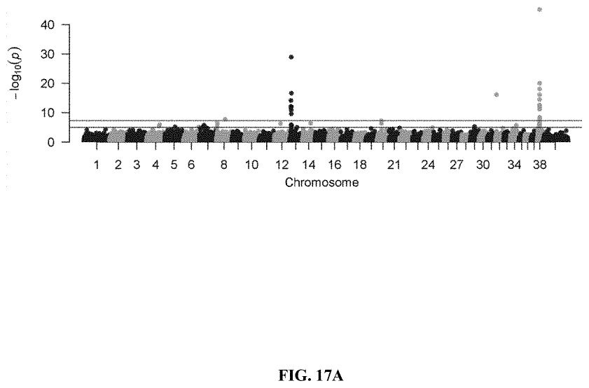

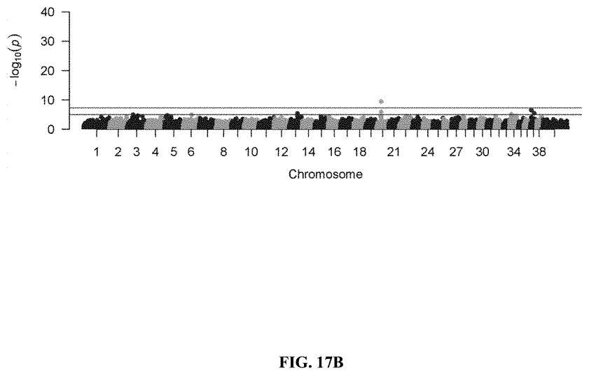

The GWAS of 358 roaned dogs (cases) and 579 non-ticked, non-roaned dogs (controls) identified three significant markers (FIG. 17A). The most significant marker was at the position 11,085,443 on CFA38, an exonic region of usherin gene (USH2A) (P=6.9×10⁻⁴⁶). The second most significant marker was on CFA13 at the position 8,625,896 that was in an intronic region of R-spondin 2 gene (RSPO2) (P=1.1×10⁻²⁹). The association with RSPO2 likely resulted from the non-roaned and roaned breeds also commonly having contrasting coat texture (e.g., English Springer Spaniels and German Wirehaired Pointers, the latter having a wiry coat, known as “furnishing”) [ref 36]. The marker at 8,625,896 on CFA13 was 15-kb away from the most likely causal variant of furnishing (167-bp insertion within the 3′UTR of RSPO2 at position 8,610,419). Similarly, the association with the marker on CFA32 at the position 4,509,367 (P=6.8×10⁴⁷) was likely due to the difference in fur length between the case and control groups, such as Border Collies (long-haired usually without roaning) and Australian Cattle Dogs (short-haired with roaning), because this marker, lying in exon 1 of fibroblast growth factor 5 (FGF5), was indeed known to be the most likely causal variant of fur length [ref 36]. To minimize the effect of genes associated with breed-specific characteristics, additional GWAS was re-run, subdividing the dataset by herding breeds (Australian Cattle Dogs, Australian Shepherds, and Border Collies) and the rest of breeds (e.g., non-herding breeds). As expected, the GWAS association signals on CFA13 and CFA32 were not consistently detected in GWAS for herding (N=268 and 495 for roaned and control dogs, respectively) and non-herding breeds (N=90 and 84 for roaned and control dogs, respectively). On the other hand, the marker on CFA38 remained highly significant in both herding and non-herding groups (P=1.5×10⁻⁷ and P=3.3×10⁻¹⁷, respectively). A weakly associated marker was found at the position 21,848,176 on CFA20, an intron of MITF (P=1.5×10⁻⁷) (FIGS. 17A-17B), and the association was more pronounced in GWAS for herding breeds (P=5.4×10⁻¹⁹). This is likely due to the difference in the frequency of S-locus alleles between breeds, where the base coat color of Australian Cattle Dogs is often mostly white with a likely genotype of s^{w}/s^{w}, whereas the piebald and Irish spotting white coat patterns are more common in Australian Shepherds and Border Collies with a likely genotype of s^{p}/− or sⁱ/− [ref 17-18].

FIGS. 17A-17B show Manhattan plots of association with roaning and ticking, including for Roaning (FIG. 17A) and Ticking (FIG. 17B). Upper and lower horizontal lines are significant (P<5×10⁻⁸) and suggestive (P<1×10⁻⁵) associations, respectively.

In contrast to the GWAS for roaning, the GWAS for the ticking pattern did not reveal strongly associated markers (N=579, 168, and 176 for controls, lightly ticked, and heavily ticked, respectively) (FIG. 17B). The same markers at MITF on CFA20 and USH2A on CFA38 were detected as marginally significant when both herding and non-herding breeds were analyzed together (P=3.5×10′ and P=3.6×10⁻⁸, respectively). However, when herding and non-herding breeds were analyzed separately, only the MITF marker was significant in herding breeds (N=75 and 495 for ticked and control dogs, respectively), whereas no marker was significantly associated with ticking in non-herding breeds (N=269 and 84 for cases and controls, respectively). Similar to roaning, this was likely due to the difference in the frequency of S-locus alleles in herding breeds since Australian Cattle Dogs were mostly ticked with large white areas in the dataset.

An 11-kilobase tandem duplication was identified. The association between the marker on CFA38 and roaning coat phenotype was robust regardless of whether herding or non-herding breeds were used, whereas this marker was not strongly associated with ticking. Thus, the analysis was focused on the region surrounding this marker to further characterize the genetic variants associated with roaning. The roan-associated region on CFA38, defined as a region containing markers with P<5.0×10′ (CFA38:10,985,456-11,380,922), had a protein-coding gene, USH2A. The non-roaned control group was completely devoid of the roan-associated “A” allele at the most significant marker at the position 11,085,443 on CFA38, while 57% and 38% of roaned dogs were AA homozygous and AG heterozygous, respectively, indicating a dominant action of this locus. A total of 321 haplotypes were identified in this region based on 52 markers, among which 21 haplotypes had the roan-associated “A” allele at the position 11,085,443. There were 16 roaned dogs (4%) without the roan-associated “A” allele (e.g., GG genotype at the position 11,085,443), five of which were homozygous across the 52 markers (three dogs with hap_GG01 homozygotes and two dogs with hap_GG02 homozygotes). The 21 haplotypes with the “A” allele shared an identical haplotype from the position 11,006,085 to 11,191,833 except for three rare haplotypes (hap_07, hap_12, and hap_21). The region between 11,006,085 and 11,072,648, where hap_07 was different from the rest of the haplotypes, was likely not involved in roaning, because there were three control dogs that were homozygous and identical to hap_07 in this region (dog_1746, dog_1758, and dog_1807). Thus, a roan-associated region was narrowed down to a 119-kb region (11,072,648-11,191,833) spanning 14 exons and 14 introns of USH2A.

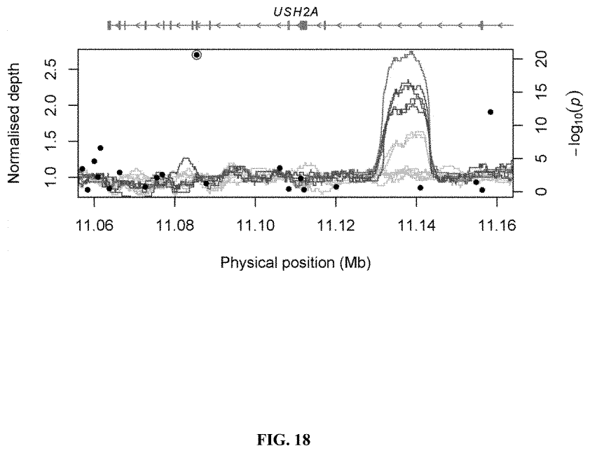

There were 4,569 previously known single nucleotide variants (SNVs) and small indels in this 119-kb region based on publicly available whole genome resequencing of 722 dogs and canid species (hereafter referred to as WGS data) [ref 31]. Variant Effect Predictor (VEP) [ref 37] identified six high-impact and 61 moderate-impact variants out of these 4,569 variants (S9 File). All 16 dogs of breeds where roaning was common were wildtype homozygotes at the six high-impact variant sites. There were three moderate-impact variants where frequencies of mutant variants were higher than 50% in the 16 dogs, one of which was the most significant GWAS marker (CFA38:11,085,443). This variant was a missense mutation (G>A) leading to an amino acid change from proline to serine of USH2A protein (FIG. 18). However, roughly 5% of roaned dogs were GG homozygotes. The remaining two moderate-impact variants were missense mutations: C>T at CFA38:11,111,286 leading to glutamic acid to lysine substitution and G>A at CFA38:11,169,445 leading to proline to serine substitution. To evaluate the association between phenotypes and genotypes of these moderate-to-high impact variants in the sample cohort, these genotypes were imputed by using IMPUTE2 [ref 38]. All 358 roaned dogs had at least one missense allele at these moderate-impact variant sites; however, there were 48 and 136 non-roaned dogs with two copies of the missense allele at these sites, respectively, indicating that these variants were unlikely to be the causal variant.

Since Variant Effect Predictor (VEP) [ref 37] indicated that none of the moderate-to-high impact variants were perfectly associated with roaning, structural variations (SVs) were next searched for as candidate causal variants for roaning by using probe intensity of the Illumina microarray (log R ratio: LRR). It was observed that the mean LRR was significantly higher in roaned dogs than control dogs at a marker position 11,140,991 on CFA38, 55-kb away from the most significant GWAS marker (probe intensity=0.171 and −0.031, N=346 and 566 for roaned and control dogs, respectively; t-test, t=29.504, P=5.2×10⁻¹³⁰). Stronger probe intensity indicated the existence of a duplicated copy of this region. There was no difference in the probe intensity at this marker between ticked and control dogs (−0.018 and −0.021, N=55 and 208 for ticked and control dogs, respectively; t-test, t=−0.858, P=0.39).

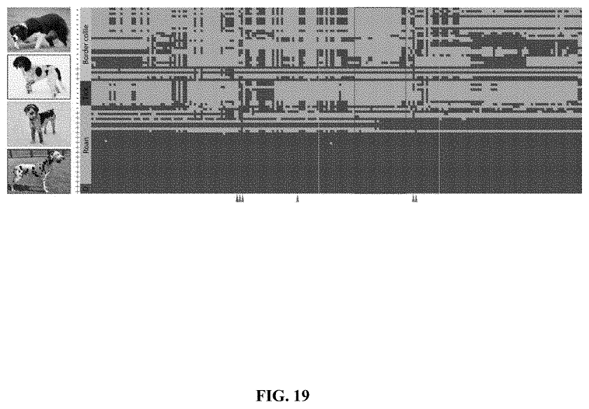

To identify a duplication associated with roaning, SVs were searched for by using the WGS data [ref 31]. Manta, an SV caller based on short-read sequence data [ref 27], identified an 11.4-kb duplication (CFA38:11,131,835-11,143,237) in 15 out of 16 dogs of breeds where roaning was common (4 Australian Cattle Dog, 1 German Wirehaired Pointer, 1 Wirehaired Pointer, and 11 Wirehaired Pointing Griffon). Five dogs in three breeds where ticking is common did not carry the duplication (1 Brittany, 1 Clumber Spaniel, and 3 English Setter), while 3 out of 15 Border Collies carried the duplication. Read depth of the dogs with the duplication was 1.5-2.5 times higher in regions between 11.13 Mb and 11.14 Mb than the genome-wide average (FIG. 18). Border Collies did not show such an abrupt increase except for the three dogs where the tandem duplication was identified by Manta. Discordant read pairs facing outwards were observed in dogs with elevated read depth in the duplicated region, suggesting that the duplication was in a tandem orientation. Out of 15 potentially roaned dogs with the duplication, 10 dogs were homozygous at the most significant GWAS marker on CFA38 and shared a long haplotype 100-kb upstream and downstream of the duplication (e.g., the duplication-associated haplotype) (FIG. 19). The remaining five dogs with the duplication had either one copy of the duplication-associated haplotype or a potential recombinant haplotype of the duplication-associated haplotype by sharing a core haplotype from the positions 11,122,646-11,167,876. The dogs without the duplication did not have the duplication-associated haplotype or similar ones. Interestingly, two Dalmatians in the WGS data were both homozygous for the duplication-associated haplotype (FIG. 19).

FIG. 18 shows normalized read depth in 5-kb sliding windows across the significant GWAS locus on CFA38 for Australian Cattle Dogs, German Wirehaired Pointer, and Border Collies. Filled circles show the corresponding markers of the Manhattan plot shown in FIG. 17A (circle: most significant marker).

FIG. 19 shows haplotypes near the marker on CFA38 significantly associated with roaning. Border Collies, breeds with high frequency of ticking, breeds with high frequency of roaning, and Dalmatians. Rows correspond to haplotypes (two rows/individual), and columns correspond to markers. The positions of the first and last markers are U.S. Pat. Nos. 11,031,835 and 11,243,237, respectively. +/−: presence and absence of the 11-kb duplication based on Manta. Red box: 11-kb duplication (CFA38:11,131,835-11,143,234). Yellow box: a core haplotype (CFA38:11,122,646-11,167,876). Red triangle: the most significant marker associated with roaning. Green triangle: markers used for defining the duplication-associated haplotypes. Photos of representative breeds are shown (from top to bottom: Border Collie, Münsterlander, German Wirehaired Pointer, and Dalmatian).

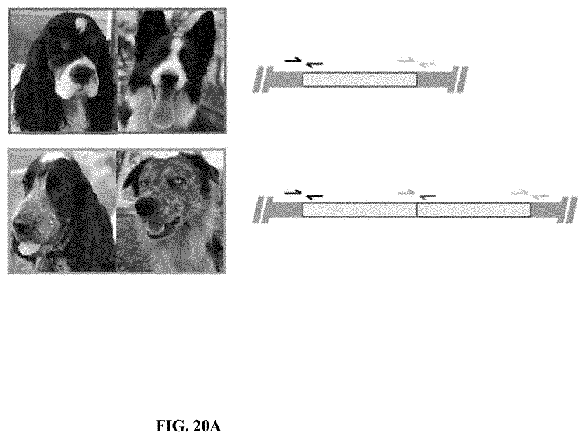

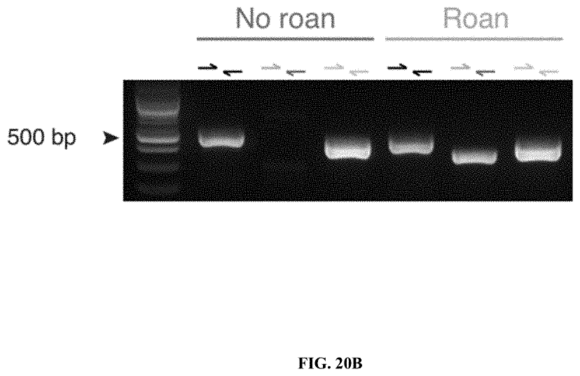

To test whether the duplication-associated haplotype identified in the WGS data was indeed associated with the tandem duplication in the GWAS discovery panel dogs, a breakpoint PCR assay was designed by targeting the region spanning the two copies (forward and reverse primers mapping to CanFam3.1 CFA38:11,143,136-11,143,155 and CFA38:11,131,969-11,131,988, respectively) (FIGS. 20A-20B). A total of 97 dogs were assayed (57 and 40 dogs with or without roaning, respectively). This primer pair produced a single 400-bp amplicon in all roaned dogs with at least one copy of the roan-associated “A” allele at the position 11,085,443 (N=52). Of the 16 roaned dogs without the roan-associated “A” allele, five samples were available for the breakpoint PCR assay. All of these five samples produced the 400-bp amplicon. There was one homozygous dog and one heterozygous dog for hap_GG01, indicating a potential recombination event between the markers at 11,120,096 and 11,140,091. Two dogs were heterozygous for hap_GG02, which was also likely a recombinant with the duplication because two other dogs homozygous for hap_GG02 were also both roaned. The fifth dog did not carry any of these two haplotypes, but one of the haplotypes, hap_r56, was identical to hap_GG02 from the position 11,076,885 to 11,156,338, indicating the presence of the duplication in this haplotype. Of the remaining 11 roaned dogs without the roan-associated “A” allele, nine dogs had at least one copy of hap_GG01 or hap_GG02, supporting the association between the duplication and roan phenotype. Two dogs without these two haplotypes (dog_1774 and dog_2063) also carried a possible recombinant haplotype: hap_r05 (identical to hap_GG01 from the position 10,985,456 to 11,298,034), and hap_r57 (identical to hap_GG02 from the position 11,072,648 to 11,380,922). Thus, it was assumed that the following haplotypes carried the duplication: all 21 haplotypes with the roan-associated “A” allele (hap_01-hap_21), hap_GG01, hap_GG02, hap_r04, hap_r05, hap_r56, hap_r57. All control dogs did not have any of these haplotypes, and none of them tested for the PCR assay produced the 400-bp amplicon (N=40).

FIGS. 20A-20B show PCR genotyping of the tandem duplication on CFA38 associated with roaning. FIG. 20A: Schematic view of the design of the PCR genotyping assay. Yellow boxes indicate the duplicated region. Single-headed arrows indicate pairs of primers to amplify three regions. The first (black) and the third (yellow) primer pairs should produce amplicons in all dogs regardless of the presence or absence of the duplication, while the second pair in the middle should produce an amplicon only in dogs carrying the duplication. Representative coat patterns of non-roaned (top) and roaned dogs (bottom) are shown (left: non-herding group, right: herding group). FIG. 20B: PCR genotyping of a roaned and control dogs. Each gel lane corresponds to PCR primer pairs depicted in panel A.

To confirm the presence of the duplication-associated haplotype that was found in two Dalmatians in the WGS data, additional data was used of 262 purebred Dalmatians genotyped by the SNP array. Two haplotypes with the “A” allele at the position 11,085,443 (hap_01 and hap_06) were common, and 98% of Dalmatians (257 dogs) had at least one copy of these haplotypes. The remaining 2% of Dalmatians (N=5) were homozygous for hap_d003, which was identical to hap_01 from the position 11,112,104 to 11,380,922. The presence of the duplication was confirmed in Dalmatians with these haplotypes (hap_01, hap_06, and/or hap_d003) by the breakpoint PCR assay (n=6). Thus, it was concluded that all 262 Dalmatians carried at least one of these haplotypes with the duplication. Since the CFA38 duplication was more strongly associated with the roaning, it was hypothesized that the duplication itself or neighboring mutations were the causal variant. The CFA38 duplication was in an intronic region of USH2A, and the orthologous region in the human reference genome (hg38) was at chr1:215,694,945-215,712,452 based on Liftover [ref 39]. At least three clusters of highly conserved sequences were identified in this region (maximum PhyloP scores of 5.5, 4.3, and 4.1), which overlapped with a DNAse I hypersensitive sites and transcription factor binding sites annotated by Open Regulatory Annotation (ORegAnno). In addition, there were two additional regions of high conservation outside the duplication (maximum PhyloP scores of 9.6 and 9.1), which were annotated as transcription factor binding sites by ORegAnno and interaction regions by GeneHancer based on Hi-C mapping.

Phenotype and genotype association were observed. The presence or absence of the tandem duplication on CFA38 was predicted for the discovery panel dogs based on the haplotypes associated with the duplication. A total of 357 dogs had at least one copy of the duplication-associated haplotypes. The presence of the duplication-associated haplotypes explained all roaned cases (246 homozygous and 112 heterozygous dogs out of 358 roaned dogs), whereas these haplotypes were absent in non-roaned dogs. These duplication-associated haplotypes were able to predict the roaned coat pattern more accurately than the genotypes of the most associated SNP in the GWAS (chi-squared test, P=3.4×10⁻²⁰⁴ for the duplication and P=1.4×10⁻¹⁸⁸ for the GWAS marker). Only 3% of ticked dogs had one copy of the duplication-associated haplotypes (11 heterozygotes and no duplication homozygote). Although the association between ticking and the duplication was significant (chi-squared test, P=5.9×10⁻⁵), this could be due to cryptic roaned fur that was not visible in the photographs.

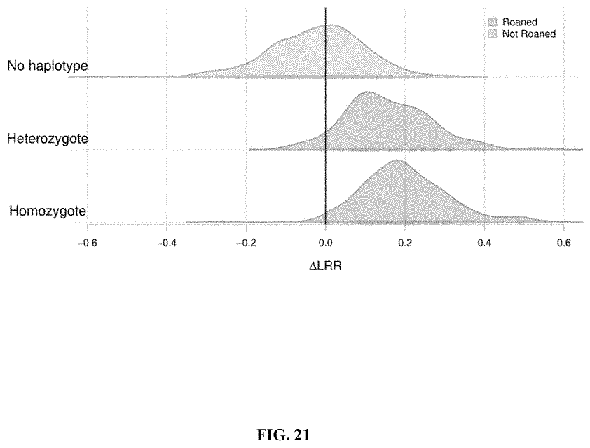

To confirm that the roaned dogs carried the CFA38 tandem duplication, the difference in LRR was compared between a marker located within the duplication (the position 11,140,991) and 10 markers in the flanking region of the duplication (ΔLRR=LRR_{inside}−LRR_{outside}) for dogs with or without roaning pattern. Despite the limited power of ΔLRR by having only one marker within the duplication, dogs with one or two copies of the duplication-associated haplotypes (e.g., heterozygotes or homozygotes) showed distinct distributions of ΔLRR, supporting the linkage between the duplication and its flanking markers (FIG. 21).

FIG. 21 shows density distribution of the array signal intensity (ΔLRR) for the discovery panel dogs with zero, one, or two copies of the duplication-associated haplotypes (no haplotype, heterozygote, and homozygote, respectively). Vertical ticks indicate individual ΔLRR of dogs with roaning (heterozygote and homozygote) and without roaning (no haplotype).

The imputed genotypes at CFA38:11,143,243 were nearly perfectly concordant with the duplication genotypes predicted by the haplotypes. All control dogs did not have the duplication (−/−) and were T/T at CFA38:11,143,243 (N=579). In roaned dogs, 246 dogs carried two copies of the duplication-associated haplotypes and were T/T at CFA38:11,143,243, and 94 dogs carried one copy of the duplication-associated haplotypes and were T/C at CFA38:11,143,243. There were five dogs where the copy number of the duplication-associated haplotypes and the imputed genotype at CFA38:11,143,243 were discordant by having one copy of the duplication-associated haplotypes but were T/T at CFA38:11,143,243 (dog_1448, dog_1685, dog_1691, dog_1981, and dog_2031). They all had one copy of haplotypes that have not been tested by the breakpoint PCR assay (hap_r04, hap_r36, and hap_r38). These untested haplotypes shared a part of haplotypes with hap_r05, hap_GG01, and hap_GG02, respectively, indicating the presence of the duplication in these recombinant haplotypes.

Prediction of roaning coat pattern was observed in the independent validation panel. An independent dataset was selected of 274 mixed breed dogs (120 roaned and 154 control dogs, respectively) to validate the association between roan coat and the markers on CFA38 (the duplication-associated haplotypes and imputed genotypes of CFA38:11,143,243). All dogs with roaning had at least one copy of the duplication-associated haplotypes and the roan-associated T allele at CFA38:11,143,243, agreeing with the pattern identified in the discovery dataset. In the control group, the duplication was absent in 97% of dogs, whereas five dogs were observed without roaned fur that were heterozygous for the duplication. ΔLRR of these five dogs were all positive (0.08-0.50), supporting the existence of the duplication in these dogs' genomes. Consistent with the duplication, all of these five dogs were C/T heterozygotes at CFA38:11,143,243 based on genotype imputation. The imputed genotypes of four dogs were confirmed by Sanger sequencing the region CFA38:11,143,161-11,143,326). They had either small spots (or ticking), faint roaning pattern in muzzle areas, a limited amount of white marking (e.g., a possible “residual white”), wolf-like sable pattern without large patches of roaning, or long fur that resulted in inaccurate phenotyping.

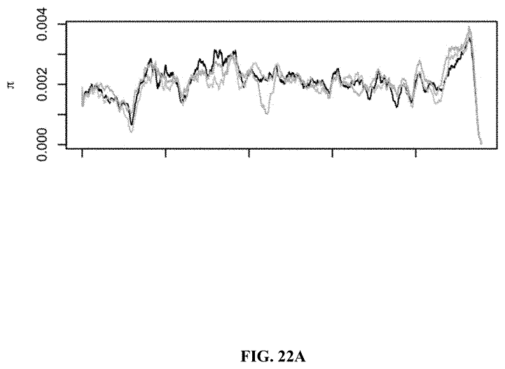

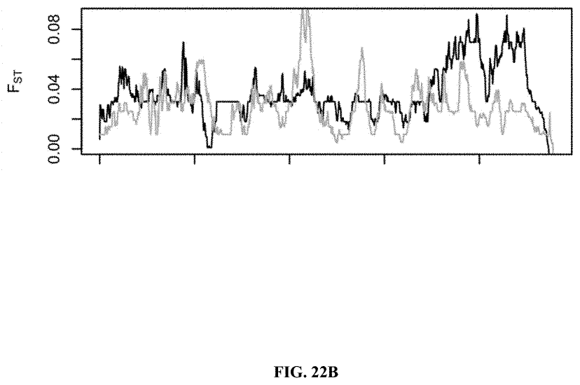

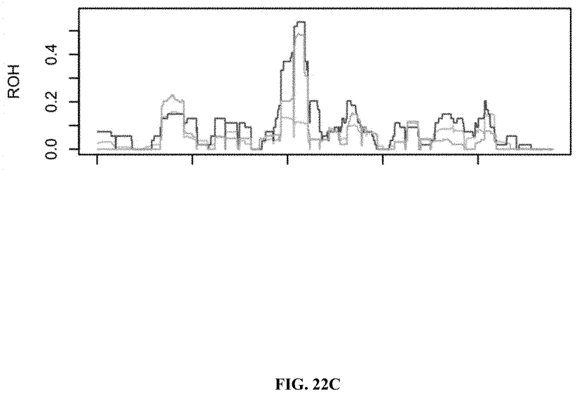

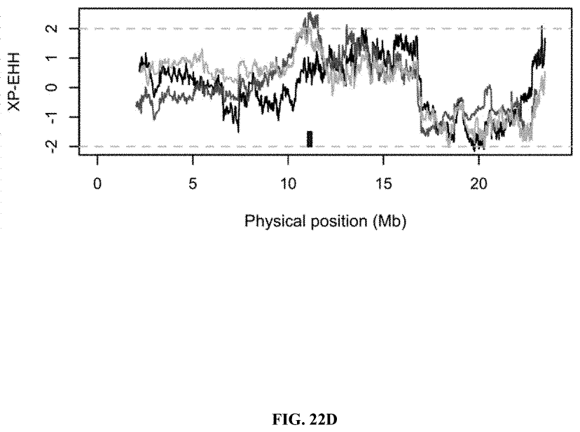

Selection on the CFA38 was observed. Whole-genome re-sequencing data showed that nucleotide diversity (π) was reduced near the duplication on CFA38 in a breed almost fixed for roaning (Wirehaired Pointing Griffon), whereas such reduction was not observed in two reference breeds, Border Collies and Labrador Retrievers (FIG. 22A). Genetic differentiation, measured as F_{ST}, was elevated in this region in the comparison between Wirehaired Pointing Griffon and Border Collies but not between Labrador Retrievers and Border Collies, suggesting selection most likely acting on the duplication (FIG. 22B). Consistent with the selection signals detected in the whole-genome data, the genotype data of the SNP array revealed that about 50% of Australian Cattle Dogs with roaned coat were autozygous between 10 and 11 Mb on CFA38 (FIG. 22C). Similarly, frequent autozygosity was found in Dalmatians but not in Border Collies in this region, indicating that the duplication-associated haplotype was likely favored by selection in Australian Cattle Dogs and Dalmatians. Moreover, cross-population extended haplotype homozygosity (XP-EHH) [refs. 33-34] showed a significant difference between Australian Cattle Dogs and Border Collies, where the maximum XP-EHH of 2.05 was found at a marker position 10,871,209 (FIG. 22D). A comparison between Dalmatians and Border Collies also showed a high XP-EHH score in this region (2.56 at the position 11,164,866), consistent with the elevated autozygosity in Dalmatians.

FIGS. 22A-22D show a signature of selection in the region on CFA38 associated with roaning. FIG. 22A: Nucleotide diversity (π) for Wirehaired Pointing Griffon (orange), Border Collies (grey), and Labrador Retriever (black) in 500-kb sliding windows. FIG. 22B: Pairwise genetic difference (F_{ST}) for Wirehaired Pointing Griffon (orange) and Labrador Retriever (black). Border Collies were used as a reference. FIG. 22C: ROH in Australian Cattle Dog (orange), Dalmatians (red), and Border Collies (grey). FIG. 22D: XP-EHH in Australian Cattle Dog (orange), Dalmatians (red), and Labrador Retrievers (black). Border Collies were used as a reference. Wirehaired Pointing Griffons and Australian Cattle Dogs are breeds where roaning is common. Blue rectangle: position of the 11-kb duplication. π and F_{ST} are estimated by using whole-genome re-sequencing data, while ROH and XP-EHH were estimated by using Embark genotyping data.

To infer the frequency of the CFA38 duplication in dog and other canine populations, the duplication-associated haplotypes were searched for, found in the discovery dataset (FIG. 19), in the WGS dataset with 722 dogs and other canid species [ref 31]. In addition to the breeds that were used for the discovery of the duplication (FIG. 19), 16 breeds had at least one copy of the duplication-associated haplotypes. These haplotypes were fairly common in some breeds, such as German Shepherds Dogs (5 out of 15 dogs) and Belgian Tervurens (4 out of 11 dogs); however, roaning, if any, should not be visible in these breeds because of the lack of white areas (e.g., S/S genotype at S-locus). The duplication-associated haplotypes were also found in breeds where roaning was occasionally observed: Portuguese Water Dogs (3 out of 11 dogs), Lagotto Romagnolos (2 out of 5 dogs), and Dachshunds (2 out of 5 dogs). Finally, village dogs in China, Papua New Guinea, and Vietnam also had the duplication-associated haplotypes (6 out of 45 dogs), indicating a potentially ancient origin of the duplication. Mapped sequence read coverage within the duplication (CFA38:11,131,835-11,143,237) was about 1.5 times and 2 times higher than the surrounding 100-kb flanking region in dogs with one or two copies of the duplication-associated haplotypes, respectively, confirming the association between the haplotypes and the duplication in these breeds. The duplication-associated haplotypes were not found in Grey Wolves (N=45).

A Dalmatian spot modifier was observed. F-locus associated with Dalmatian's spots has been shown to be genetically linked to SLC2A9 on CFA3, a gene responsible for a high level of uric acid excretion in the urine (hyperuricosuria) [refs. 8, 19]. The segregation pattern of the Dalmatian-like spots in a backcross population of Dalmatians and English Pointers suggests that this phenotype is recessive: F/− with flecking (scattered white furs within pigmented region) and f/f without flecking [ref 8]. Given the presence of the duplication at the R-locus on CFA38 in both Dalmatians and roaned dogs, it was assumed that the recessive f allele was fixed in Dalmatians but was rare in roaned dogs. To identify genomic regions associated with the Dalmatian's spot, genotypes were imputed of 604,843 SNVs on CFA3 reported in Plassais et al. [ref 31] for 358 roaned dogs in the discovery dataset and 262 Dalmatians. An SNV at CFA3:72,316,930, which was 2.9 Mb away from the causal variant of hyperuricosuria (CFA: 69,456,869), showed the largest genotype frequency difference between Dalmatians and roaned dogs, where 98% of dalmatians and 2% of roaned dogs were AA homozygotes (chi square test, p=1×10¹²⁰). Six Dalmatians were GA heterozygotes, and none of them were homozygous for the reference G allele. This marker was in an intronic region of the Ras Homolog Family Member H (RHOH) gene.

To test if the candidate marker CFA3:72,316,930 was associated with Dalmatian-like spots, 43 mixed breed dogs were additionally genotyped with 1) Dalmatian-like spots without flecking or other types of spots with flecking, 2) white background coat with possibly s^{w}/s^{w} extreme white genotype at S-locus, and 3) at least one copy of the duplication-associated haplotype on CFA38. Seven dogs had typical Dalmatian-like spots in most of their bodies, while the remaining 36 dogs had black or brown ticks smaller than typical Dalmatian spots usually with some white hairs interspersed within the ticks. Similar to the purebred Dalmatians, imputed genotypes at CFA3:72,316,930 of seven mixed breed dogs with Dalmatian-like spots were all AA homozygous. However, four dogs without Dalmatian-like spots also had AA homozygous genotype. SVs in this region were not found in the two Dalmatians in the WGS dataset.

A novel locus was observed to be associated with roaning but not strongly with ticking. It has been debated whether T-locus for ticking and R-locus for roaning have a similar molecular basis since the mid 20th century [ref 40]. Although the results of this study did not yield genomic regions associated with ticking, a duplication at 11.13-11.14 Mb on CFA38 was shown to be strongly associated with roaning, indicating that these two theoretical loci are likely located in different genomic regions. Owing to the extended linkage in the surrounding region of the duplication, haplotypes with the duplication were able to be identified. The presence of the duplication in these haplotypes was confirmed by the breakpoint PCR assay, Sanger sequencing of the PCR amplicon spanning the duplication midpoint, and whole-genome re-sequencing data for the identification of discordant read pairs and abrupt read depth increase. In addition, the distribution of the array signal intensity in dogs with 0, 1, or 2 copies of the duplication-associated haplotypes was in agreement with the expected distribution. This mutation is nearly completely penetrant by explaining more than 99% of roaning cases in both purebred and mixed breed dogs. Thus, the haplotype-based linkage test can accurately detect the presence of the CFA38 duplication, which has high predictability for the roaning coat pattern. The strong association between the phenotype and the imputed SNP genotype at CFA38:11,143,243, which is tightly linked with the duplication, further supports the involvement of this region in this coat pattern.

Roaning was found in dogs with one or two copies of the duplication-associated haplotypes, suggesting that the CFA38 duplication is dominant in its effect on fur pigmentation. Pigmented fur in a roaned coat is intermingled with unpigmented fur, which is associated with certain S-locus variants, including the SINE insertion and variable length polymorphisms in the putative promoter region of MITF on CFA20 [refs. 17-18]. Although the SNP genotyping array platform could not unambiguously distinguish all of these S-locus variants, the presence of all three genotypes at the SINE indel marker (CFA20:21,836,232) in roaned dogs indicates that the association between roaning and the duplication on CFA38 is robust regardless of the type of white spotting patterns (e.g., Irish spotting, piebald, and extreme white). A total of five non-roaned dogs were found with one copy of the duplication-associated haplotypes in the validation panels. Four of them (dog_10056, dog_10079, dog_10087, and dog_10166) had a long coat, which makes it difficult to accurately distinguish between ticked and roaned patterning, while the remaining dog had limited white spotting patterns (dog_10028). The small white spotting pattern is likely a residual white, which were excluded from the study. Assuming that the phenotypes of these dogs were correctly assigned, there might be additional modifier loci interacting with R-locus and/or S-locus.

The VEP analysis did not find any SNVs and indels that may potentially be functionally relevant to pigmentation in the roan-associated 119-kb region. This left the 11-kb tandem duplication as the strongest candidate for roaning, but follow-up functional validation may be performed to investigate the exact impact of this duplication on the development of pigmented fur in otherwise unpigmented area. The comparative analysis identified putative regulatory regions within and nearby the duplication that are highly conserved in vertebrates. USH2A encodes the protein usherin. In humans, USH2A mutations are associated with Usher syndrome, characterized by progressive hearing loss and vision impairment often accompanied by retinitis pigmentosa [ref 41]. A functional assay by using USH2A knockout mice showed that this gene is involved in the maintenance of retinal photoreceptors and the development of cochlear (inner ear) hair cells [ref 42]. A study showed that a mutation in USH2A showed abnormal pigment deposition and reduced expression of MITF and other melanin metabolism-related genes, such as tyrosinase (TYR) and oculocutaneous albinism II (OCA2) genes, in retinal cells derived from induced pluripotent stem cells, indicating a potential involvement of USH2A in the pigmentation pathway [ref 43]. Interestingly, the distribution of usherin in healthy individuals is highly conserved between mice and humans, in which skin was completely devoid of this protein [refs. 44-45]. The duplication of the putative regulatory regions may result in ectopic expression of USH2A in skin melanocytes. Alternatively, the duplication may facilitate alternative splicing and create a novel protein isoform, since this complex gene with 73 exons may form several isoforms [refs. 41 and 46]. High prevalence of congenital deafness in Dalmatians, Australian Cattle Dogs, and other commonly roaned breeds [ref 47] may also indicate a potential pleiotropic effect of USH2A on both hearing and pigmentation [ref 48].

An evolutionary origin of roaning was observed. The strong selection signals in the surrounding region of the CFA38 duplication indicate that the frequency of the duplication increased during the formation of modern breeds (FIGS. 22A-22D). In the discovery panel of dogs, the duplication is common in Australian Cattle Dog (94%), Cesky Fousek (100%), English Cocker Spaniel (100%), German Shorthaired Pointer (73%), German Wirehaired Pointer (100%), Spinone Italiano (100%), and Wirehaired Pointing Griffon (100%). In addition, the duplication-associated haplotypes were found in other distantly-related breeds (e.g., German Shepherd Dogs and Portuguese Water Dogs) and village dogs (e.g., indigenous dogs that accompany humans but are not selectively bred), indicating that selection acted on a variation that existed in the ancestral canine population (e.g., “soft sweep”).

Because of the high prevalence of the CFA38 duplication in Dalmatians, it was hypothesized that Dalmatian's spots have a similar genetic basis with roaning, while the interaction between the CFA38 duplication and F-locus on CFA3 localizes the distribution of pigmented fur. This is in line with the development pattern of spots in Dalmatians and roaning, both of which emerge between two to eight weeks after birth. While an involvement of uncharacterized T-locus may not be ruled out, the shared haplotype on CFA38 (FIG. 19) and a nested phylogenetic position of Dalmatians within commonly roaned breeds (e.g., English Cocker Spaniel and German Wirehaired Pointer) [ref 49] indicates that Dalmatian's spots have been likely derived from roaned ancestors.

The Australian Cattle Dog, as its name suggests, was established in Australia in the 19th century by crossing some Collie-type dogs with Dingos (a wild dog in Australia), Bull Terriers, Kelpies and Dalmatians [ref 50]. Therefore, the duplication-associated haplotypes on CFA38 may have been introgressed from Dalmatians to the ancestral population of Australian Cattle Dog during its breed formation. Following selective breeding for roaning likely increased the frequency of the duplication in the Australia Cattle Dog population.

In conclusion, a potential involvement of USH2A in coat pigmentation in dogs was demonstrated, by using genome-wide SNP array genotyping and customer-provided photographs. An 11-kb duplication in the intronic region of this gene was observed to be strongly associated with roaned coat in both purebred and mixed breed dogs. Since there was no association signal near KITLG on CFA15, these results confirmed that KITLG was not responsible for roaning in dogs [ref 9]. This study provided an example of phenotypic convergence where roaned coat has evolved independently in dogs, horses, cattles, among others by using different genes. The similar genetic makeup of Dalmatian's spots and roaning in other breeds affirms the important role of epistatic interaction in the evolution of novel phenotype. In addition, a tentative causal mutation lies in a non-coding region which may modify expression patterns of USH2A. The study highlights the importance of epistatic interactions and rewiring regulatory networks as a contributor to a burst of phenotypic divergence.

The materials, methods, and results of this Example are described by, for example, Kawakami et al., “R-locus for roaned coat is associated with a tandem duplication in an intronic region of USH2A in dogs and also contributes to Dalmatian spotting,” PLOS ONE, Mar. 23, 2021, which is incorporated by reference herein in its entirety.

### REFERENCES

[Ref 1] Jackson I J. Molecular and developmental genetics of mouse coat color. Annu Rev Genet. 1994; 28: 189-217. doi:10.1146/annurev.ge.28.120194.001201 is incorporated by reference herein in its entirety.
[Ref 2] Manceau M, Domingues V S, Linnen C R, Rosenblum E B, Hoekstra H E. Convergence in pigmentation at multiple levels: mutations, genes and function. Philos Trans R Soc Lond B Biol Sci. 2010; 365: 2439-2450. doi:10.1098/rstb.2010.0104 is incorporated by reference herein in its entirety.
[Ref 3] Liu S, Lorenzen E D, Fumagalli M, Li B, Harris K, Xiong Z, et al. Population genomics reveal recent speciation and rapid evolutionary adaptation in polar bears. Cell. 2014; 157: 785-794. doi:10.1016/j.cell.2014.03.054 is incorporated by reference herein in its entirety.
[Ref 4] Haase B, Brooks S A, Schlumbaum A, Azor P J, Bailey E, Alaeddine F, et al. Allelic heterogeneity at the equine kit locus in dominant white (w) horses. PLoS Genet. 2007; 3. doi:10.1371/journal.pgen.0030195 is incorporated by reference herein in its entirety.
[Ref 5] Mariat D, Taourit S, Guérin G. A mutation in the MATP gene causes the cream coat colour in the horse. Genet Sel Evol GSE. 2003; 35: 119-133. doi:10.1186/1297-9686-35-1-119 is incorporated by reference herein in its entirety.
[Ref 6] Finch J, Abrams S, Finch A. Analogs of human genetic skin disease in domesticated animals. Int J Womens Dermatol. 2017; 3: 170-175. doi:10.1016/j.ijwd.2017.01.003 is incorporated by reference herein in its entirety.
[Ref 7] Landi M T, Bauer J, Pfeiffer R M, Elder D E, Hulley B, Minghetti P, et al. MC1R germline variants confer risk for BRAF-mutant melanoma. Science. 2006; 313: 521-522. doi:10.1126/science.1127515 is incorporated by reference herein in its entirety.
[Ref 8] Schaible R H. Linkage of a pigmentary trait with a high level of uric acid excretion in the dalmatian dog. Genetics. 1976; 83: S68 is incorporated by reference herein in its entirety.
[Ref 9] Schmutz S M, Berryere T G. Genes affecting coat colour and pattern in domestic dogs: a review. Anim Genet. 2007; 38: 539-549. doi:10.1111/j.1365-2052.2007.01664.x is incorporated by reference herein in its entirety.
[Ref 10] Charlier C, Denys B, Belanche J I, Coppieters W, Grobet L, Mni M, et al. Microsatellite mapping of the bovine roan locus: A major determinant of White Heifer Disease. Mamm Genome. 1996; 7: 138. doi:10.1007/s003359900034 is incorporated by reference herein in its entirety.
[Ref 11] Seitz J J, Schmutz S M, Thue T D, Buchanan F C. A missense mutation in the bovine MGF gene is associated with the roan phenotype in Belgian Blue and Shorthorn cattle. Mamm Genome Off J Int Mamm Genome Soc. 1999; 10: 710-712. doi:10.1007/s003359901076 is incorporated by reference herein in its entirety.
[Ref 12] Fontanesi L, D'Alessandro E, Scotti E, Liotta L, Crovetti A, Chiofalo V, et al. Genetic heterogeneity and selection signature at the KIT gene in pigs showing different coat colours and patterns. Anim Genet. 2010; 41: 478-492. doi:10.1111/j.1365-2052.2010.02054.x is incorporated by reference herein in its entirety.
[Ref 13] Cho I-C, Zhong T, Seo B-Y, Jung E-J, Yoo C-K, Kim J-H, et al. Whole-genome association study for the roan coat color in an intercrossed pig population between Landrace and Korean native pig. Genes Genomics. 2011; 33: 17-23. doi:10.1007/s13258-010-0108-4 is incorporated by reference herein in its entirety.
[Ref 14] Talenti A, Bertolini F, Williams J, Moaeen-Ud-Din M, Frattini S, Coizet B, et al. Genomic analysis suggests KITLG is responsible for a roan pattern in two Pakistani goat breeds. J Hered. 2018; 109: 315-319. doi:10.1093/jhered/esx093 is incorporated by reference herein in its entirety.
[Ref 15] Kaelin C B, Barsh G S. Genetics of pigmentation in dogs and cats. Annu Rev Anim Biosci. 2013; 1: 125-156. doi:10.1146/annurev-animal-031412-103659 is incorporated by reference herein in its entirety.
[Ref 16] Schmutz S M, Berryere T G, Dreger D L. MITF and white spotting in dogs: a population study. J Hered. 2009; 100: S66—S74. doi:10.1093/jhered/esp029 is incorporated by reference herein in its entirety.
[Ref 17] Karlsson E K, Baranowska I, Wade C M, Salmon Hillbertz N H C, Zody M C, Anderson N, et al. Efficient mapping of mendelian traits in dogs through genome-wide association. Nat Genet. 2007; 39: 1321-1328. doi:10.1038/ng.2007.10 is incorporated by reference herein in its entirety.
[Ref 18] Körberg I B, Sundström E, Meadows J R S, Pielberg G R, Gustafson U, Hedhammar A, et al. A simple repeat polymorphism in the MITF-m promoter is a key regulator of white spotting in dogs. PLOS ONE. 2014; 9: e104363. doi:10.1371/journal.pone.0104363 is incorporated by reference herein in its entirety.
[Ref 19] Bannasch D, Safra N, Young A, Karmi N, Schaible R S, Ling G V. Mutations in the SLC2A9 Gene Cause Hyperuricosuria and Hyperuricemia in the Dog. PLOS Genet. 2008; 4: e1000246. doi:10.1371/journal.pgen.1000246 is incorporated by reference herein in its entirety.
[Ref 20] Clark L A, Wahl J M, Rees C A, Murphy K E. Retrotransposon insertion in SILV is responsible for merle patterning of the domestic dog. Proc Natl Acad Sci USA. 2006; 103: 1376-1381. doi:10.1073/pnas.0506940103 is incorporated by reference herein in its entirety.
[Ref 21] Hédan B, Cadieu E, Botherel N, Dufaure de Citres C, Letko A, Rimbault M, et al. Identification of a Missense Variant in MFSD12 Involved in Dilution of Phaeomelanin Leading to White or Cream Coat Color in Dogs. Genes. 2019; 10. doi:10.3390/genes10050386
[Ref 22] Sponenberg D, Rothschild M. Genetics of coat colour and hair texture. In: Ruvinsky A, Sampson J, editors. The Genetics of the Dog. Wallingford, U K: CABI; 2001. pp. 61-85 is incorporated by reference herein in its entirety.
[Ref 23] Chang C C, Chow C C, Tellier L C, Vattikuti S, Purcell S M, Lee J J. Second-generation PLINK: rising to the challenge of larger and richer datasets. GigaScience. 2015; 4: 7. doi:10.1186/s13742-015-0047-8 is incorporated by reference herein in its entirety.
[Ref 24] Zhou X, Stephens M. Genome-wide efficient mixed-model analysis for association studies. Nat Genet. 2012; 44: 821-824. doi:10.1038/ng.2310 is incorporated by reference herein in its entirety.
[Ref 25] Browning S R, Browning B L. Rapid and accurate haplotype phasing and missing-data inference for whole-genome association studies by use of localized haplotype clustering. Am J Hum Genet. 2007; 81: 1084-1097. doi:10.1086/521987 is incorporated by reference herein in its entirety.
[Ref 26] Auton A, Rui Li Y, Kidd J, Oliveira K, Nadel J, Holloway J K, et al. Genetic recombination is targeted towards gene promoter regions in dogs. PLoS Genet. 2013; 9: e1003984. doi:10.1371/journal.pgen.1003984 is incorporated by reference herein in its entirety.
[Ref 27] Chen X, Schulz-Trieglaff O, Shaw R, Barnes B, Schlesinger F, Kälberg M, et al. Manta: rapid detection of structural variants and indels for germline and cancer sequencing applications. Bioinforma Oxf Engl. 2016; 32: 1220-1222. doi:10.1093/bioinformatics/btv710 is incorporated by reference herein in its entirety.
[Ref 28] Li H, Durbin R. Fast and accurate long-read alignment with Burrows-Wheeler transform. Bioinforma Oxf Engl. 2010; 26: 589-595. doi:10.1093/bioinformatics/btp698 is incorporated by reference herein in its entirety.
[Ref 29] McKenna A, Hanna M, Banks E, Sivachenko A, Cibulskis K, Kernytsky A, et al. The Genome Analysis Toolkit: a MapReduce framework for analyzing next-generation DNA sequencing data. Genome Res. 2010; 20: 1297-1303. doi:10.1101/gr.107524.110 is incorporated by reference herein in its entirety.
[Ref 30] Robinson J T, Thorvaldsdóttir H, Winckler W, Guttman M, Lander E S, Getz G, et al. Integrative genomics viewer. Nat Biotechnol. 2011; 29: 24-26. doi:10.1038/nbt.1754 is incorporated by reference herein in its entirety.
[Ref 31] Plassais J, Kim J, Davis B W, Karyadi D M, Hogan A N, Harris A C, et al. Whole genome sequencing of canids reveals genomic regions under selection and variants influencing morphology. Nat Commun. 2019; 10: 1489. doi:10.1038/s41467-019-09373-w is incorporated by reference herein in its entirety.
[Ref 32] Danecek P, Auton A, Abecasis G, Albers C A, Banks E, DePristo M A, et al. The variant call format and VCFtools. Bioinforma Oxf Engl. 2011; 27: 2156-2158. doi:10.1093/bioinformatics/btr330 is incorporated by reference herein in its entirety.
[Ref 33] Sabeti P C, Varilly P, Fry B, Lohmueller J, Hostetter E, Cotsapas C, et al. Genome-wide detection and characterization of positive selection in human populations. Nature. 2007; 449: 913-918. doi:10.1038/nature06250 is incorporated by reference herein in its entirety.
[Ref 34] Gautier M, Vitalis R. rehh: an R package to detect footprints of selection in genome-wide SNP data from haplotype structure. Bioinforma Oxf Engl. 2012; 28: 1176-1177. doi:10.1093/bioinformatics/bts115 is incorporated by reference herein in its entirety.
[Ref 35] Sams A J, Boyko A R. Fine-scale resolution of runs of homozygosity reveal patterns of inbreeding and substantial overlap with recessive disease genotypes in domestic dogs. G3 Bethesda Md. 2019; 9: 117-123. doi:10.1534/g3.118.200836 is incorporated by reference herein in its entirety.
[Ref 36] Cadieu E, Neff M W, Quignon P, Walsh K, Chase K, Parker H G, et al. Coat variation in the domestic dog is governed by variants in three genes. Science. 2009; 326: 150-153. doi:10.1126/science.1177808 is incorporated by reference herein in its entirety.
[Ref 37] McLaren W, Gil L, Hunt S E, Riat H S, Ritchie G R S, Thormann A, et al. The Ensembl Variant Effect Predictor. Genome Biol. 2016; 17: 122. doi:10.1186/s13059-016-0974-4 is incorporated by reference herein in its entirety.
[Ref 38] Howie B N, Donnelly P, Marchini J. A Flexible and Accurate Genotype Imputation Method for the Next Generation of Genome-Wide Association Studies. PLOS Genet. 2009; 5: e1000529. doi:10.1371/journal.pgen.1000529 is incorporated by reference herein in its entirety.
[Ref 39] Hinrichs A S, Karolchik D, Baertsch R, Barber G P, Bejerano G, Clawson H, et al. The UCSC Genome Browser Database: update 2006. Nucleic Acids Res. 2006; 34: D590-598. doi:10.1093/nar/gkj144 is incorporated by reference herein in its entirety.
[Ref 40] Little C C. The inheritance of coat color in dogs. Ithaca, N.Y.: Comstock; 1957 is incorporated by reference herein in its entirety.
[Ref 41] Adato A, Lefèvre G, Delprat B, Michel V, Michalski N, Chardenoux S, et al. Usherin, the defective protein in Usher syndrome type IIA, is likely to be a component of interstereocilia ankle links in the inner ear sensory cells. Hum Mol Genet. 2005; 14: 3921-3932. doi:10.1093/hmg/ddi416 is incorporated by reference herein in its entirety.
[Ref 42] Liu X, Bulgakov O V, Darrow K N, Pawlyk B, Adamian M, Liberman M C, et al. Usherin is required for maintenance of retinal photoreceptors and normal development of cochlear hair cells. Proc Natl Acad Sci USA. 2007; 104: 4413-4418. doi:10.1073/pnas.0610950104 is incorporated by reference herein in its entirety.
[Ref 43] Guo Y, Wang P, Ma JI-1, Cui Z, Yu Q, Liu S, et al. Modeling retinitis pigmentosa: retinal organoids generated from the iPSCs of a patient with the USH2A mutation show early developmental abnormalities. Front Cell Neurosci. 2019; 13. doi:10.3389/fnce1.2019.00361 is incorporated by reference herein in its entirety.
[Ref 44] Pearsall N, Bhattacharya G, Wisecarver J, Adams J, Cosgrove D, Kimberling W. Usherin expression is highly conserved in mouse and human tissues. Hear Res. 2002; 174: 55-63. doi:10.1016/50378-5955(02)00635-4 is incorporated by reference herein in its entirety.
[Ref 45] Bhattacharya G, Miller C, Kimberling W J, Jablonski M M, Cosgrove D. Localization and expression of usherin: a novel basement membrane protein defective in people with Usher's syndrome type IIa. Hear Res. 2002; 163: 1-11. doi:10.1016/s0378-5955(01)00344-6 is incorporated by reference herein in its entirety.
[Ref 46] Aller E, Jaijo T, Beneyto M, Nájera C, Oltra S, Ayuso C, et al. Identification of 14 novel mutations in the long isoform of USH2A in Spanish patients with Usher syndrome type II. J Med Genet. 2006; 43: e55. doi:10.1136/jmg.2006.041764 is incorporated by reference herein in its entirety.
[Ref 47] Strain G M. Deafness prevalence and pigmentation and gender associations in dog breeds at risk. Vet J. 2004; 167: 23-32. doi:10.1016/51090-0233(03)00104-7 is incorporated by reference herein in its entirety.
[Ref 48] Vasiliadis D, Metzger J, Distl O. Demographic assessment of the Dalmatian dog—effective population size, linkage disequilibrium and inbreeding coefficients. Canine Med Genet. 2020; 7: 3. doi:10.1186/s40575-020-00082-y is incorporated by reference herein in its entirety.
[Ref 49] Parker H G, Dreger D L, Rimbault M, Davis B W, Mullen A B, Carpintero-Ramirez G, et al. Genomic analyses reveal the influence of geographic origin, migration, and hybridization on modern dog breed development. Cell Rep. 2017; 19: 697-708. doi:10.1016/j.celrep.2017.03.079 is incorporated by reference herein in its entirety.
[Ref 50] Coile C. Encyclopedia of Dog Breeds. 3rd ed. Hauppauge, N.Y.: Barron's Educational Series; 2015. Available: www.amazon.com/Encyclopedia-Breeds-Caroline-Coile-Ph-D/dp/0764167294/ref=sr_1_1?keywords=Encyclopedia+of+Dog+Breeds.&qid=1584557529&sr=8-1 is incorporated by reference herein in its entirety.

### Example 4— Five Genetic Variants Explain Over 70% of Hair Coat Pheomelanin Intensity Variation in Purebred and Mixed Breed Domestic does

In mammals (e.g., dogs), the pigment molecule pheomelanin may confer red and yellow color to hair, and the intensity of this coloration may be caused by variation in the amount of pheomelanin. Domestic dogs may exhibit a wide range of pheomelanin intensity, ranging from the white coat of the Samoyed to the deep red coat of the Irish Setter. While several genetic variants may be associated with specific coat intensity phenotypes in certain dog breeds, they may not explain the majority of phenotypic variation across breeds. In order to gain further insight into the extent of multigenicity and epistatic interactions underlying coat pheomelanin intensity in dogs, a large dataset obtained via a direct-to-consumer canine genetic testing service was leveraged. The database comprised genome-wide single nucleotide polymorphism (SNP) genotype data and owner-provided photos for 3,057 pheomelanic mixed breed and purebred dogs from 62 breeds and varieties spanning the full range of canine coat pheomelanin intensity. First, a genome-wide association study (GWAS) was performed on 2,149 of these dogs to search for additional genetic variants that underlie intensity variation. GWAS identified five loci significantly associated with intensity, of which two (CFA15 29.8 Mb and CFA20 55.8 Mb) are consistent with literature and three (CFA2 74.7 Mb, CFA18 12.9 Mb, CFA21 10.9 Mb) have not previously been reported. In order to assess the combined predictive power of these loci across dog breeds, the GWAS data set was used to construct a trained linear classifier, which explained over 70% of variation in coat pheomelanin intensity in an independent validation dataset of 908 dogs. These results introduce three novel pheomelanin intensity loci, and further demonstrate the multigenic nature of coat pheomelanin intensity determination in domestic dogs.

For thousands of years, humans have selectively bred domestic dogs for desired physical and behavioral phenotypes, including a wide variety of coat colors and patterns [refs. 1-2]. For example, historical writings indicate that shepherds from as early as the first century AD preferred white-colored herding and livestock guardian dogs because this coloration allowed them to quickly distinguish their dogs from wolves [ref 3], while some modern sporting breeds such as Chesapeake Bay Retrievers have been selectively bred to have dark to light brown coats “colored to match their working environment” [ref 4]. Indeed, nearly all modern breed standards published by various kennel clubs provide detailed specifications on coloration. Genetic mapping studies have identified several key genes that account for much of the coat color and patterning variation across domestic dog breeds [refs. 5-16], but the genetic bases of some common phenotypes may remain unclear.

Canine coat colors and patterns may result from varied expression of two pigment molecules: eumelanin, which is black or brown, and pheomelanin which is reddish-yellow. Most canids have coats containing a mixture of hairs expressing eumelanin, pheomelanin, or both, but many domestic dogs have coats in which only pheomelanin is expressed. These “pheomelanic” coats result from mutations in and around one of two genes that regulate switching between eumelanin and pheomelanin synthesis in hair follicle melanocytes: melanocortin 1 receptor (MC1R, known as the “E locus”) and agouti signaling protein (ASIP, known as the “A locus”) [14]. At least four different recessive mutations in and around the MC1R gene inhibit the synthesis of eumelanin in hair follicle melanocytes, resulting in a solid “recessive red” coat containing only pheomelanin [refs. 5-7 and 17]. A completely or mostly red coat can also result from carrying a dominant ASIP variant (AY), which produces “sable” coats with varying amounts of black/brown hairs concentrated around the dorsal midline, and pheomelanic hairs across the rest of the body [refs. 8 and 15].

The intensity of pheomelanic coloration may vary widely across and within breeds that are fixed for recessive red or sable coats. For example, Irish Setters have consistently deep red coats, while Soft-coated Wheaten Terriers have coats that vary from cream to tan. Additionally, many breeds with solid white or cream coats have been shown to be recessive red, including Bichon Frisé, Samoyed, West Highland White terrier, and White German Shepherd [refs. 5 and 18]. Uncovering the genetic basis of pheomelanin intensity variation in dogs may be unexpectedly challenging. It was originally hypothesized that extreme pheomelanin dilution in pheomelanic dogs—resulting in a white or cream colored coat—was primarily controlled by a single locus [refs. 19-20], as it is in several other mammalian species [refs. 21-30]. However, it is increasingly apparent that even this one extreme of coat pheomelanin intensity is a multigenic trait across, and perhaps within, dog breeds.

Three recent studies have identified several genetic variants that are able to explain some coat pheomelanin intensity variation in certain breeds. The first study identified two variants in and upstream of the MC1R gene that are highly predictive of extreme pheomelanin dilution in recessive red Siberian Huskies and Australian Cattle Dogs [ref 17], but did not investigate how these variants affect coat pheomelanin intensity in other breeds. A second study identified a missense mutation in the major facilitator superfamily domain containing 12 gene (MFSD12) that is associated with extreme pheomelanin dilution in a wide variety of breeds [ref 18]. However, dogs that were homozygous for the mutation still showed variation in pheomelanin dilution within some breeds, suggesting that pheomelanin dilution is a multigenic trait both across and within breeds. Similarly, a third study identified a copy number variant upstream of the KIT ligand gene (KITLG) that was predictive of red intensity in Nova Scotia Duck Tolling Retriever and Poodle [ref 31], but not in two of the most common (in the United States [ref 32]) and phenotypically variable breeds: Golden Retriever and Labrador Retriever. In this study, the genetic underpinnings of coat pheomelanin intensity variation in dogs were investigated by testing whether there are additional loci that affect intensity across dog breeds, and investigating how these loci might interact. This was achieved by performing a genome-wide association study (GWAS), which identified five genomic regions that are significantly associated with coat pheomelanin intensity, and showing that these loci are able to explain approximately 70% of variation in coat pheomelanin intensity in mixed breed and purebred dogs.

Participating dogs were part of a veterinary customer base. Owners provided informed consent to use their dogs' data in scientific research by agreeing the following statement: “I want this dog's data to contribute to medical and scientific research”. Ethical approval was not required as non-invasive methods for genotype or phenotype collection were used (buccal swabbing and photographing, respectively). Dogs were never handled directly by researchers. Owners were given the opportunity to opt-out of the study at any time during data collection. The discovery and validation cohorts were selected from data available collected between October 2018 and June 2020. All data were de-identified.

Genotype and phenotype data were collected as follows. Cheek cell samples were collected by dog owners with buccal swabs, and DNA was extracted (using methods by Illumina) and genotyped at 214,634 biallelic autosomal and X chromosome markers on an Embark Veterinary custom Illumina CanineHD SNP array. Dogs that had been genotyped between October 2018 and June 2020 were filtered to those that 1) had owner consent to use of their genetic data and owner-reported data for research, 2) had at least one owner-provided photo, 3) had owner reported breed assignments, and 4) were genetically “recessive red” (e/e at the E locus [ref 6]) or “sable” (ky/ky at the K locus and Ay/Ay, At, Aw, or a at the A locus [ref 8]) per their array genotypes. Of the 3,596 dogs that met these four criteria, 72 were excluded from further analysis due to discrepancy between genetic analysis and owner-reported breed, leaving 3,524 to be phenotyped. Breed assignments and genotypes were determined at the E, K, and A loci for the 3,057 dogs that passed subsequent quality control steps.

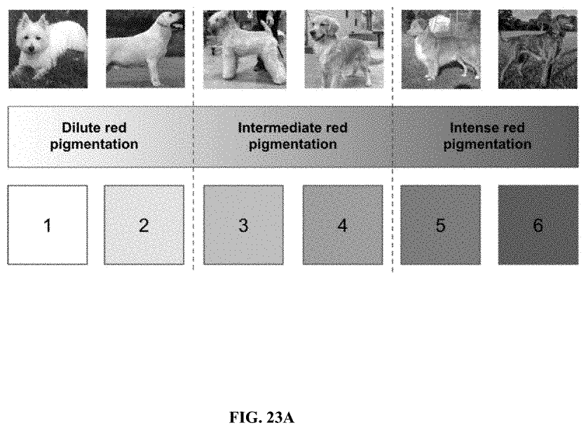

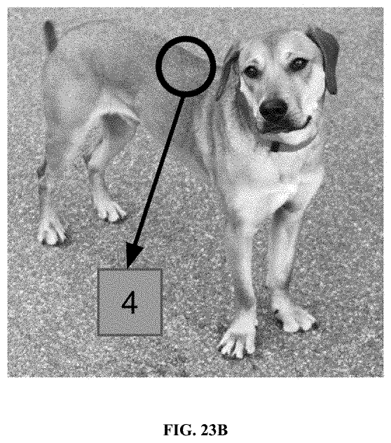

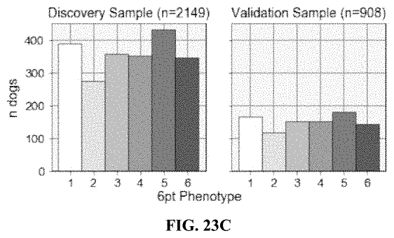

Phenotyping was performed as follows. To develop a color scale for visual phenotyping, three shades (cream, tan, and red) were selected that encompass the range of coat pheomelanin intensity phenotypes in domestic dogs, their hexadecimal values (#FFFEF9, #D3A467, and #93471A) were obtained. Then, the Matplotlib [33] LinearSegmentedColormap and Normalize functions were used to obtain six equally spaced hexadecimal values spanning the range of values defined by these three colors. The six point coat color scale (FIG. 23A) includes the colors encoded by these hexadecimal values: #FFFEF9 (1), #EDDABF (2), #DCB684 (3), #C69158 (4), #AD6C39 (5), and #93471A (6).

FIGS. 23A-23C show the six point coat pheomelanin intensity scale. FIG. 23A: Photos of six purebred dogs that exhibit the full range of coat pheomelanin intensity in canids are shown above a continuous color scale and numbered swatches showing the color of each of the six phenotype values used in this study. From left to right, the breeds of the dogs in these photos are: West Highland White Terrier, Yellow Labrador Retriever, Soft-coated Wheaten Terrier, Golden Retriever, Nova Scotia Duck-Tolling Retriever, Irish Setter. All six dogs pictured were part of the study sample. FIG. 23B: An example of a dog that displays “countershading”. The black circle indicates the part of the photo that was used to assign this dog's phenotype (4 on the six point color scale), which in this case was the mid back. FIG. 23C: Histograms showing the number of dogs with each phenotype value in the discovery and validation samples.

To assign coat color phenotypes to dogs, a single scientist visually evaluated owner-provided photos and assigned each dog to one of the six levels in the coat color scale or excluded it from further analysis. To account for red countershading—meaning darker red hair along the back, ears, and the tip of the tail in some breeds (FIG. 23B)—all dogs were typed based on their coat color at the top of the mid back, or if the back could not be clearly seen, the top of the head. The pheomelanin intensity phenotype could not be confidently typed based on available photos for 215 dogs (due to poor photo quality, positioning of the dog in the photo, multiple dogs shown in the same photo, or lack of red hair on the head or shoulders due to coat patterning) and these were excluded from further analyses.

In order to achieve a more balanced distribution of phenotypes across the GWAS sample, concordant owner-reported and genetically-determined breed assignments were used to identify an additional 192 genetically pheomelanic, purebred dogs with no owner-provided photo that belonged to breeds that are fixed for red coats (5 or 6 on the phenotype scale). These dogs were assigned the most common six-point phenotype value in their breed across the rest of the sample. The dogs phenotyped in this manner consisted of 21 Brittanys, 2 Ibizan Hounds, 4 Irish Red and White Setters, 8 Redbone Coonhounds, 138 Rhodesian Ridgebacks, 16 Vizslas, and 3 Welsh Springer Spaniels. Including these, the dataset comprised 3,501 dogs with confident phenotype and breed assignments.

To assess phenotyping consistency, 350 dogs with photos were randomly selected using the pandas DataFrame.sample( ) method [ref 34] and re-phenotyped on the six point scale by the same scientist who performed the original phenotyping. The concordance between the original and new phenotypes was 97%, and 100% of dogs had a new phenotype value that was within 1 point of their original phenotype value.

Genotype data were filtered as follows. PLINK 1.9 [ref 35] was used to remove array markers with >5% missingness (n=10,063) and dogs with >3% missingness (n=3) across the remaining markers. Then, 441 close relatives were removed from the remaining dogs by identifying pairs of dogs with pi_hat≥0.45 (calculated using PLINK 1.9's—genome utility) and dropping the dog with the higher genome-wide missingness in each pair from the dataset. After these steps, the mean individual genotyping rate was 99.9% across 204,571 markers in 3,057 dogs from 62 different breeds and varieties.

Discovery and validation data partitioning were performed as follows. The 3,057 dogs were grouped according to their breed, each breed was subsetted by six point phenotype value, and each phenotype group was split randomly 70:30 into the discovery and validation datasets using the pandas DataFrame.sample( ) method [ref 34]. As a result, the breed ancestry and phenotype (FIG. 23C) distributions were highly similar between the discovery and validation datasets, with both datasets having at least one individual from each of the 62 breeds. The discovery dataset partitions were combined (n=2,149) and used as input to the discovery GWAS, then used as a training dataset to define marker weights in the predictive models. The validation data partitions were combined (n=908) and used to assess the accuracy of the predictive model (see “Predictive models for coat pheomelanin intensity” below).

Genome-wide association was performed as follows. To identify genomic regions associated with pheomelanin intensity variation, coat color was encoded as both a case-control (cream versus red) and quantitative trait (six point scale), and a multivariate linear mixed model was constructed using GEMMA v.0.98 [ref 36] to the discovery dataset. To further account for confounding effects of shared ancestry among dogs of the same or closely related breeds, kinship matrices were constructed from array genotypes using the GEMMA -gk command and used as a random effect in the model for each GWAS run. Setting GEMMA's -miss and -maf values to 0.05 and 0.001 led to 16,343 markers being excluded from analysis, for a total of 188,288 markers in 2,149 dogs. The association result files were generated by GEMMA. In all GWAS, the Bonferroni correction was used with an alpha of 0.05 as a threshold for considering a SNP to be significant at the genome-wide level.

An initial GWAS run showed marginally significant associations in the MC1R and RSPO2 genes on canine chromosome (CFA) 5 and CFA13, respectively. It was determined that these signals were likely driven by differences across phenotype groups that are not directly related to coat pheomelanin intensity. Specifically, dogs with lower intensity phenotype values are enriched for breeds that are fixed for the recessive red genotype at MC1R gene on CFAS [ref 6], and breeds with a high frequency of “furnishings” caused by a mutation in RSPO2 [ref 37]. To account for this, dogs' genotypes were included at the top CFAS and 13 markers (CFAS: 63,694,334 and CFA13: 8,611,728, respectively) as covariates in the GWAS models which eliminated these association signals. The association results produced by the GWAS models are discussed herein, including these covariates.

Due to the difficulty of obtaining appropriate hair samples for the thousands of dogs in the sample from individual owners, the amount of pheomelanin in dogs' hair coats was not measured (as done in [ref 31]). Because of this, the assumption that the phenotype values were truly quantitative was not tested. To account for the possibility that treating the phenotype values as quantitative might create spurious associations, a case-control GWAS was performed contrasting cream (phenotype value 1 or 2) and red (phenotype value 5 or 6) dogs. The case-control and quantitative GWAS detected the same set of top markers, so the quantitative GWAS results are focused on in the results herein.

Analysis of public whole genome sequencing data was performed as follows. Raw whole genome paired-end short read sequencing datasets were downloaded as fastq files from the Sequence Read Archive [ref 38] (Suppl. Data File 3) and aligned to the canFam3.1 reference genome using the BWA-MEM algorithm inBWA version 0.7.17 [ref 39]. The mapped reads were filtered and soft-clipped using the Picard Tools version 2.21.4 [ref 40] CleanSam tool, then converted to sorted and indexed .bam files using samtools. Duplicate reads were identified and removed using the Picard Tools MarkDuplicates tool. For regions of interest, the mean depth of sequencing coverage across all autosomes was calculated using the Genome Analysis Toolkit 3 [ref 41] DepthOfCoverage tool, and depth of coverage values in regions of interest were divided by the mean autosomal depth of coverage to obtain normalized depth of coverage values.

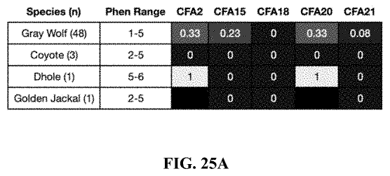

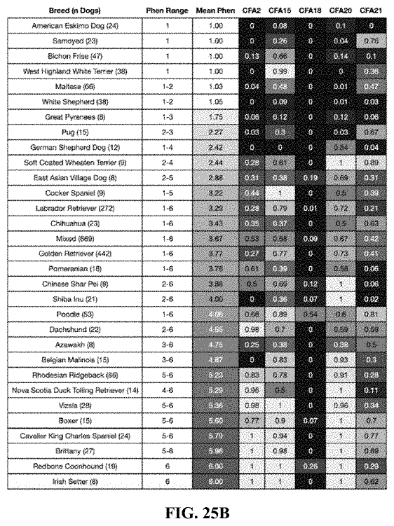

To determine which allele at each top GWAS marker was most likely the ancestral allele, genotypes were obtained at these markers across 54 publicly available wild canid whole genome sequencing datasets downloaded from the Sequence Read Archive [ref 38] (48 Gray Wolves, 3 Coyotes, 1 Dhole, and 1 Golden Jackal). The accession information for these 54 datasets and their genotypes at the top GWAS markers are available in ( ). The allele frequencies at the top GWAS markers in these populations are shown in FIG. 25A.

Testing for epistatic interactions among GWAS hits was performed as follows. The PLINK 1.9 [ref 35]—epistasis tool was used to test for epistasis among pairs of the top five GWAS variants in the discovery sample. This tool was used to train a multivariate linear regression classifier model, given by Y=f30+f31gA+f32gB+f33gAgB for each variant pair (A, B), where Y is the quantitative phenotype value, gA and gB are allele counts, f31 and f32 are the effects sizes of variants A and B, 133 is the effect size of the interaction between A and B, and 130 is a random effect. Interactions with a p-value of <0.05 were considered to be statistically significant.

Estimation of dominance effects was performed as follows. To evaluate the dominance relationship between the alleles at each of the top GWAS SNPs, predicted heterozygote phenotype values were estimated under complete additivity as the midpoint of the standardized six point phenotype values in the two homozygote classes [ref 42]. Then the dominance effect d was estimated for each SNP as the difference between the observed and expected mean phenotype values in the heterozygote class. A positive value of d is consistent with the red-associated allele being at least partially dominant, and a negative value of d is consistent with the red-associated allele being at least partially recessive. Values of d were considered to be statistically significant if the 95% confidence interval of the observed heterozygote mean phenotype did not include the additive heterozygote midpoint phenotype.

Predictive models for coat pheomelanin intensity were constructed as follows. Using the linear_model module in the Python scikit-learn package version 0.21.3 [ref 43], a multivariate linear regression classifier model was trained on the training set of discovery cohort dogs with coat color phenotypes as the dependent variable. In these models, the independent variables were genotype dosage values (coded additively, or with one allele completely dominant to the other) at the five top GWAS markers, as well terms representing their pairwise interactions (e.g., the product of the dosage values at the two individual loci). The coefficients, standard error, t-test values for each independent variable, as well as the y-intercept, adjusted R-squared, and log likelihood values for the best fit model are provided in Table 17.

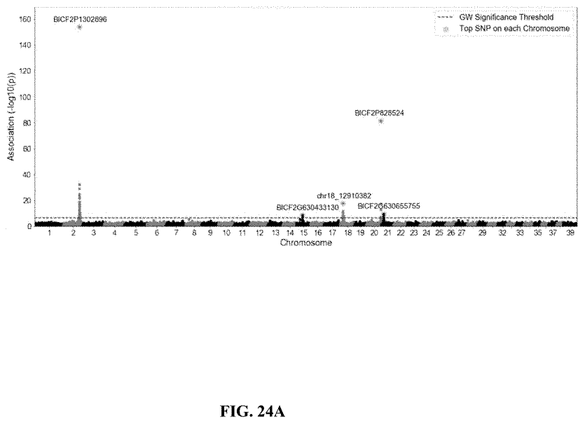

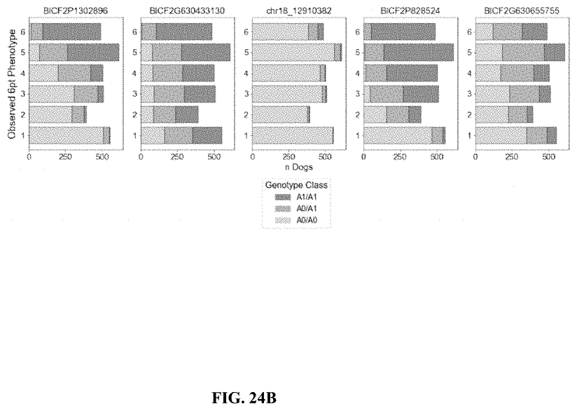

Results from the GWAS identified five loci associated with coat pheomelanin intensity variation. GWAS treating coat pheomelanin intensity phenotypes as a quantitative trait in the discovery dataset identified five significantly associated genomic regions on CFA2, 15, 18, 20, and 21. A total of 88 SNPs passed the Bonferroni correction threshold of 2.73×10⁻⁷ (6.56 on the −log₁₀ scale) (supp data). The most strongly associated markers in these regions were CFA2: 74,746,906 base pairs (bp) (BICF2P1302896), CFA15: 29,840,789 bp (BICF2G630433130), CFA18: 12,910,382 bp (chr18_12910382), CFA20: 55,850,145 bp (BICF2P828524), and CFA21: 10,864,834 bp (BICF2G630655755) (FIGS. 24A-24B, Table 15).

FIGS. 24A-24B show quantitative coat pheomelanin intensity GWAS results. FIG. 24A: GWAS p-values are shown in a Manhattan plot for the autosomes (chromosome 1-38) and the X chromosome (chromosome 39). For each chromosome with one or more genome-wide significant markers, the top marker on the chromosome is highlighted in gold and labeled with its marker ID. The blue dashed line shows the minimum unadjusted −log₁₀ (p-value) for genome-wide significance using the Bonferroni correction: 6.56. FIG. 24B: Bar plots show the number of dogs with each phenotype value (1-6) for each genotype class at each of the top five GWAS markers. The genotype classes are coded according to the dosage of the red-associated alleles at each marker, which are listed in Table 15 as “Allele 1”.

TABLE 15
Top GWAS markers at five associated loci

| | | | Red | | −log₁₀ | |
|---|---|---|---|---|---|---|
| | canFam3.1 | | Allele, | Beta, | (Wald's | |
| Marker ID | Pos | Gene | Freq. | se | p-value) | PVE |
| BICF2P1302896 | 2: 74,746,906 | lincRNA (exonic) | A, 0.42 | 0.95, 0.03 | 153.83 | 0.28 |
| BICF2G630433130 | 15: 29,840,789 | Intergenic | G, 0.66 | 0.23, 0.04 | 8.60 | 0.02 |
| chr18_12910382 | 18: 12,910,382 | SLC26A4 (exonic) | G, 0.05 | 0.88, 0.1 | 17.76 | 0.04 |
| BICF2P828524 | 20: 55,850,145 | Intergenic | G, 0.65 | 0.78, 0.04 | 81.45 | 0.16 |
| BICF2G630655755 | 21: 10,864,834 | TYR (intronic) | A, 0.38 | 0.23, 0.04 | 9.51 | 0.02 |

Table 15: Marker IDs, physical position in the canFam3.1 reference genome, gene symbol (if applicable), the red-associated allele and its frequency (Red Allele, Freq.), effect size (Beta) and standard error (se) of the effect size, uncorrected −log₁₀ (Wald's p-value), and proportion of variance explained (PVE) for the most significant marker at each of the five associated loci.

The locations of these markers relative to annotated canFam3.1 functional elements, as well as r2 between genotypes at each top GWAS variant and neighboring variants (e.g., linkage disequilibrium), were determined,

FIGS. 25A-25B show species and breed allele frequencies at top GWAS markers. FIG. 25A: shows the frequencies of the red-associated allele at the top five GWAS markers in 53 public wild canid genomes [ref 34]. FIG. 25B: shows the same information across 31 breeds with at least 8 individuals in the GWAS sample. Each row shows the breed/species phenotype value range and (for phenotyped dogs, e.g., the dogs in the GWAS sample) the mean phenotype value for each breed, with the mean phenotype value colored by the corresponding coat color. The remaining columns show the breed/species allele frequencies (blue=lower allele frequency, yellow=higher allele frequency, black=no data) of the red-associated alleles at each of the top five GWAS markers, which are labelled according to their chromosome number. Mean phenotype and allele frequency values are colored white or black to improve readability.

Further, three novel regions associated with coat pheomelanin intensity were identified. The CFA2, 18, and 21 associations with coat pheomelanin intensity may not have been previously reported. The top CFA2 variant, BICF2P1302896, falls within the second exon of the long intergenic non-coding RNA (lincRNA) ENSCAFG00000042716 at CFA2: 74,744,598-74,747,735 bp. At this marker, the cream-associated allele was more common in Gray Wolves, but the red-associated allele was present in the single Dhole, making it difficult to infer which allele is ancestral (FIG. 25A). The red-associated allele was present in most of the domestic dog breeds examined, but it was only fixed in breeds with consistently high coat pheomelanin intensity such as Brittany, Redbone Coonhound, and Irish Setter (FIG. 25B). The cream-associated allele was fixed in several breeds that are fixed for completely cream coats, including American Eskimo Dog, Samoyed, West Highland White Terrier, and White Shepherd (FIG. 25B).

The top CFA18 variant, chr18_12910382, is a missense mutation p.I487M in a conserved residue of the twelfth exon of the solute carrier family 26 member 4 gene (SLC26A4). Unlike the top CFA2 GWAS marker, the wild canid genomes examined only carried the cream-associated allele at this marker, indicating that the red-associated allele is most likely derived and possibly dog-specific (FIG. 25A).

The top CFA21 variant, BICF2G630655755, falls within the second intron of the tyrosinase gene (TYR). At this marker, only the cream-associated allele was present in the outgroup wild canids (Golden Jackal and Dhole). Although both alleles were present in Gray Wolves, the cream-associated allele is more common and therefore most likely ancestral (FIG. 25A). In domestic dogs, both alleles were present in most breeds (FIG. 25B).

Further, two top associations were observed. The top CFA15 variant, BICF2G630433130, is located approximately 8 kilobases (kb) downstream of a 6 kb copy number variant (CNV) near the KIT ligand gene (KITLG) that was previously associated with variation in coat pheomelanin intensity in Nova Scotia Duck Tolling Retrievers and Poodles [ref 31], as well as squamous cell carcinoma of the digit in eumelanistic, but not recessive red, Standard Poodles [ref 44]. The red-associated allele at this marker was present at an intermediate frequency (23%) across 48 Gray Wolves, but not in Coyote, Dhole, or Golden Jackal (FIG. 25A). Consistent with Weich et al. [ref 31], the red-associated variant segregates at high frequencies in breeds that consistently have high coat pheomelanin intensity but is also segregating at high frequencies in some breeds that are fixed for extreme pheomelanin dilution, such as West Highland White Terrier (FIG. 25B).

The top CFA20 variant is the same variant reported in another coat pheomelanin intensity GWAS using over 90 different breeds, which was used to fine map the peak to a nearby missense mutation in the major facilitator superfamily domain containing 12 gene (MFSD12) at CFA20: 55,856,000 bp [ref 18]. It was observed that the red-associated allele at BICF2P828524 was segregating at an intermediate frequency in Gray Wolves and carried by the single Dhole for which that data was available, but absent in 3 Coyotes genomes, making it difficult to infer which allele is ancestral. Consistent with Hédan et al. [ref 18], the red-associated allele was more common across domestic dogs than the cream-associated allele, and while the cream-associated allele was far more common in breeds that are fixed for extreme pheomelanin dilution, it was rarely fixed in those breeds (FIG. 25B).

Most of the dogs in the GWAS sample were not directly genotyped at CFA20: 55,856,000 bp or the CFA15 CNV upstream of KITLG. To evaluate the extent to which the top CFA15 marker is predictive of copy number at the CFA15 CNV, publicly available whole genome short-read sequence datasets was obtained for 33 dogs of various breeds from the Sequence Read Archive [ref 38], and for each dog, the average read depth was calculated across the CNV base pair range and its genotype was obtained at BICF2G630433130. The number of red-associated alleles at BICF2G630433130 correlated with a higher mean read depth across the CNV range (Kruskal Wallis test, p-value=2.21×10⁻⁴), indicating that the GWAS signal at BICF2G630433130 is likely associated with this CNV.

Of the 2,149 dogs in the discovery dataset, 988 were run on a version of the genotyping array that included both BICF2P828524 and a new marker at CFA20: 55,856,000 bp (these genotypes are included in ( )). Across these dogs, the overall r² between genotypes at the two markers was 0.78. Thus, it was concluded that the GWAS signal observed at BICF2P828524 is likely primarily or solely driven by the previously identified missense mutation in MFSD12.

Further, a relationship between associated QTL and coat pheomelanin intensity was observed. Within the GWAS sample, several breeds with consistently cream or red coats showed complete fixation of the cream- or red-associated allele (respectively) of at least one marker (FIG. 25B). However, no combination of variants was necessary or sufficient to completely explain coat pheomelanin intensity across all breeds.

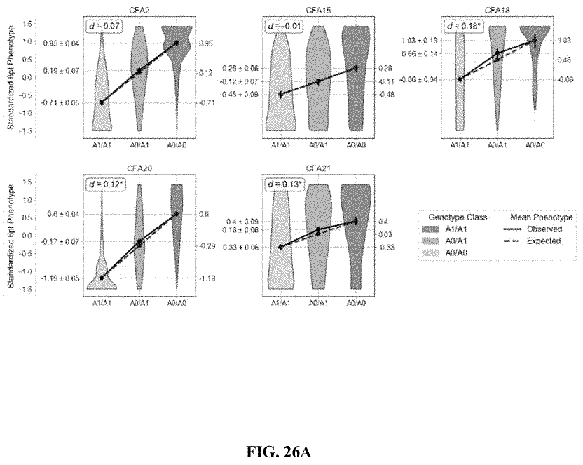

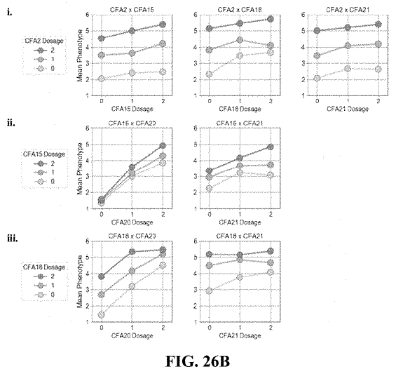

Dominance effects were observed as follows. For each of the top GWAS SNPs (which is referred to by their chromosome number herein), the dominance effect d was estimated as the difference between the observed and expected mean standardized six point phenotype value for the heterozygote class (Methods) (FIG. 26A).

FIGS. 26A-26B show dominance and epistatic interactions. FIG. 26A: For each of the top five GWAS markers, violin plots show the distribution of observed normalized six point phenotype values for each genotype class. The black lines connect the observed means of the three genotype classes, and the blue lines connect the expected means under a perfectly additive model. The estimated dominance coefficient for each marker, d, is shown in the upper left hand corner of each plot. An asterisk indicates that the predicted heterozygote class mean phenotype fell outside the 95% confidence interval of the observed heterozygote mean phenotype, which indicates that d is statistically significant. FIG. 26B: Scatter plots showing genotype-phenotype interactions at the seven locus pairs that showed statistically significant interaction effects per the epistasis test. In each plot, the “dosage”, e.g., the diploid genotype coded as the number of red-associated alleles, is displayed on the X axis, and the dosage at the other marker is represented by the three lines connecting the points. The Y axis shows the mean 6 point coat pheomelanin intensity phenotype across dogs with each genotype combination.

It was observed that the expected heterozygote mean phenotypes under additivity at the top CFA2 and 15 SNPs fell within the 95% confidence intervals of the observed heterozygote mean phenotypes, indicating that these loci behave in a mostly additive manner. At the top CFA18, 20, and 21 SNPs, the mean heterozygote phenotypes were significantly higher than the additive expectations, indicating that the red-associated alleles at these loci are at least partially dominant to the cream-associated alleles.

Epistatic interaction was observed as follows. When pairwise tests for epistatic interaction were applied to the top five GWAS variants, seven pairs of variants showed statistically significant interactions: CFA15×CFA20, CFA18×CFA20, CFA2×CFA15, CFA18×CFA21, CFA2×CFA18, CFA2×CFA21, and CFA15×CFA21 (Table 16).

TABLE 16
Pairwise tests for epistatic interaction among top GWAS markers

| Interaction | β3 | STAT | p-value |
|---|---|---|---|
| CFA15 × CFA20 | 0.216 | 41.835 | 9.98 × 10₁₁ * |
| CFA18 × CFA20 | 0.426 | 15.652 | 7.62 × 10₋₅ * |
| CFA2 × CFA15 | −0.150 | 12.569 | 3.92 × 10₋₄ * |
| CFA18 × CFA21 | −0.471 | 12.019 | 5.27 × 10₋₄ * |
| CFA2 × CFA18 | −0.310 | 7.409 | 6.49 × 10₋₃ * |
| CFA2 × CFA21 | −0.098 | 5.815 | 1.59 × 10₋₂ * |
| CFA15 × CFA21 | −0.145 | 5.542 | 1.86 × 10₋₂ * |
| CFA20 × CFA21 | −0.066 | 2.459 | 1.17 × 10⁻¹ |
| CFA2 × CFA20 | 0.049 | 1.945 | 1.63 × 10⁻¹ |
| CFA15 × CFA18 | −0.144 | 0.559 | 4.55 × 10₋₁ |

Table 16: Interaction term coefficients (β3), test statistic, and p-value for each pair of the top five GWAS variants. Interactions with a p-value<5×10⁻² (marked with an asterisk) were considered statistically significant.

Two locus genotype and phenotype combinations for these variant pairs are shown in FIG. 26B. The top CFA2 variant exhibits weak negative epistasis with the red-associated alleles at CFA15, 18, and 21 (shown in (i)). Two copies of the cream associated allele at the top CFA20 variant almost entirely masks the effect of the red-associated allele at the top CFA15 variant, and the top CFA15 variant exhibits negative epistasis with the top CFA21 variant (shown in (ii)). The top CFA18 variant exhibits positive epistasis with the top CFA20 variant and negative epistasis with the top CFA21 variant (shown in (iii)).

Further, a multi-locus linear classifier model was constructed and trained to determine coat pheomelanin intensity with high accuracy. In agricultural, livestock, and canine genetics [refs. 45-48], a common approach for accurately predicting multigenic trait phenotypes such as body weight is to fit a statistical model with phenotype as a function of genotypes at multiple genetic markers. For traits with a significant genetic variance component, a model fit on a sufficiently large and representative training sample can be used to accurately predict phenotypes for new individuals given their genotypes without knowing the true underlying genetic architecture of the trait. The phenotypic predictions produced by these models can then be used to learn more about the genetic architecture of the trait. To assess the predictive value of the five associated loci and potential epistatic interactions, a series of multiple linear regression classifier models were trained using genotype values at the top CFA2, 15, 18, 20, and 21 GWAS markers as independent variables.

First, a machine learning classifier model was trained on normalized six point phenotype values that split the genotypes at all five loci into two variables each indicating whether or not they were heterozygous (“1”), and whether or not they were homozygous for the red-associated allele (“2”). The ratios of the model coefficients (f3) for the 1 and 2 variables at each locus provided an additional evaluation of the dominance relationship between the two alleles: loci for which the 1 f3 was approximately half of the 2 f3 fit the assumption of additivity, whereas loci for which the 1 f3 was approximately zero were more consistent with the red-associated allele being recessive to the other allele, and loci for which the 1 and 2 f3s were similar were more consistent with the red-associated allele being dominant to the other allele. Based on the f3 values for this model (Table 17), it was concluded that the CFA2 and 20 loci explain more variance when coded as additive, the CFA15 locus explains more variance when the red-associated allele is coded as recessive, and the CFA18 and 21 loci explain more variance when the red-associated allele is coded as dominant. These findings broadly agree with the analysis of dominance effects at each locus shown in FIGS. 26A-26B.

TABLE 17
Evaluating additivity at top GWAS markers using
linear model coefficients for heterozygotes
versus red-associated allele homozygotes

| Variable | β | Std err | t-value | P > \|t\| | PRE |
|---|---|---|---|---|---|
| Intercept | −1.504 | 0.033 | −45.080 | <2.2 × 10₋₁₆ | — |
| CFA2_1 | 0.472 | 0.029 | 16.016 | <2.2 × 10₋₁₆ | 0.107 |
| CFA_2 | 1.068 | 0.030 | 35.313 | <2.2 × 10₋₁₆ | 0.369 |
| CFA15_1 | 0.057 | 0.033 | 1.718 | 8.6 × 10₋₂ | 0.001 |
| CFA15_2 | 0.208 | 0.032 | 6.499 | <2.2 × 10₋₁₆ | 0.019 |
| CFA18_1 | 0.234 | 0.049 | 4.759 | <2.2 × 10₋₁₆ | 0.011 |
| CFA18_2 | 0.208 | 0.082 | 2.542 | 1.1 × 10₋₂ | 0.003 |
| CFA20_1 | 0.700 | 0.034 | 20.859 | <2.2 × 10₋₁₆ | 0.169 |
| CFA20_2 | 1.232 | 0.032 | 39.036 | <2.2 × 10₋₁₆ | 0.417 |

Table 17: Coefficients, coefficient standard error, t score values, and t test p-values for the y-intercept and each of the independent variables in a predictive model that encodes each dog's genotype at each of the five top GWAS markers according to whether or not it was heterozygous (“1”), and whether or not it was homozygous for the red-associated allele (“2”). For each of the independent variables, the proportional reduction of error (PRE) value is also shown. PREs represent the fraction of the total sum of squares error that is accounted for by each independent variable.

Next, five machine learning classifier models were trained with six point phenotype values as a function of genotype at each locus using its best dominance encoding in order to estimate the predictive power of each locus individually. This showed that the CFA2 and CFA20 loci each explained over 50% of the variance in six point phenotypes, while the CFA15, 18, and 21 loci each explained less than 10% of the variance (Table 18).

TABLE 18
Predictive power of individual loci

| Locus | Model | Ajd. _{R}_{²} | ln(likelihood) |
|---|---|---|---|
| CFA2 | y = 2.364 + | 0.501 | −3,462.569 |
| | 1.435 * CFA2 | | |
| CFA15 | y = 3.13 + | 0.063 | −4,142.560 |
| | 0.865 * CFA15_2 | | |
| CFA18 | y = 3.448 + | 0.047 | −4,160.124 |
| | 1.413 * CFA18_red_dom | | |
| CFA20 | y = 1.603 + | 0.508 | −3,447.096 |
| | 1.512 * CFA20 | | |
| CFA18 | y = 2.996 + | 0.077 | −4,120.061 |
| | 0.969 * CFA21_red_dom | | |

Table 18: Best fit linear regression model equations, adjusted R-squared, and log likelihood scores are shown for each of the individual top GWAS SNPs using the dominance encoding most supported by the data in Table 17. The “CFA15_2” term encodes CFA15 genotype assuming that the red-associated allele is completely recessive, e.g., 1 if homozygous for the red-associated allele, and 0 if either of the other two genotype classes. The “CFA18_red_dom” and “CFA21_red_dom” terms encode CFA18 and CFA21 genotypes assuming that the “CFA21_red_dom” terms encode CFA18 and CFA21 genotypes assuming that the red-associated allele is completely dominant, e.g., 1 if heterozygous or homozygous for the red-associated allele, and 0 if homozygous for the other allele.

To quantitatively determine the best combination of dominance encodings in a multilocus model, 31 machine learning classifier models were trained with each possible combination of the additive and most likely dominance encoding at all five loci. A model treating all five loci as completely additive was able to explain 73% of variation in the six point phenotype (adjusted R-squared=0.730) (Table 19, Section A). The dominance model with the best fit (adjusted R-squared=0.7324) coded the red allele at CFA15 as recessive (“CFA15_2”), the red alleles at CFA18 and CFA21 as dominant (“CFA18_red_dom”, “CFA21_red_dom”), and CFA2 and CFA20 as additive (Table 19, Section B).

TABLE 19
Comparison of multilocus coat pheomelanin intensity predictive models

| | Variables | β ± se | t-value | P > \|t\| | PRE | Adj. R² | ln(likelihood) |
|---|---|---|---|---|---|---|---|
| A. | Intercept | 1.012 ± 0.049 | 20.831 | <2.2 × 10₋₁₆ | — | 0.7300 | −2,795.30 |
| | CFA2 | 0.915 ± 0.026 | 35.074 | <2.2 × 10⁻¹⁶ | 0.365 | | |
| | CFA15 | 0.191 ± 0.026 | 7.225 | <2.2 × 10⁻¹⁶ | 0.024 | | |
| | CFA18 | 0.272 ± 0.056 | 4.85 | <2.2 × 10⁻¹⁶ | 0.011 | | |
| | CFA20 | 1.038 ± 0.026 | 39.262 | <2.2 × 10⁻¹⁶ | 0.419 | | |
| | CFA21 | 0.215 ± 0.027 | 0.027 | <2.2 × 10⁻¹⁶ | 0.029 | | |
| B. | Intercept | 1.074 ± 0.043 | 25.088 | <2.2 × 10₋₁₆ | — | 0.7324 | −2,785.92 |
| | CFA2 | 0.920 ± 0.026 | 35.666 | <2.2 × 10⁻¹⁶ | 0.373 | | |
| | CFA15_2 | 0.286 ± 0.039 | 7.256 | <2.2 × 10⁻¹⁶ | 0.024 | | |
| | CFA18_red_dom | 0.405 ± 0.074 | 5.444 | <2.2 × 10⁻¹⁶ | 0.014 | | |
| | CFA20 | 1.037 ± 0.026 | 39.453 | <2.2 × 10⁻¹⁶ | 0.421 | | |
| | CFA21_red_dom | 0.355 ± 0.040 | 8.904 | <2.2 × 10⁻¹⁶ | 0.036 | | |
| C. | Intercept | 1.606 ± 0.062 | 25.834 | <2.2 × 10₋₁₆ | — | 0.5394 | −3375.46 |
| | CFA15_2 | 0.053 ± 0.096 | 0.550 | 5.82 × 10⁻¹ | 0.000 | | |
| | CFA15_2 × CFA20 | 0.374 ± 0.063 | 5.956 | <2.2 × 10⁻¹⁶ | 0.016 | | |
| | CFA20 | 1.290 ± 0.043 | 29.844 | <2.2 × 10⁻¹⁶ | 0.294 | | |
| D. | Intercept | 1.095 ± 0.054 | 20.250 | <2.2 × 10₋₁₆ | — | 0.7353 | −2772.11 |
| | CFA2 | 0.908 ± 0.026 | 35.087 | <2.2 × 10⁻¹⁶ | 0.366 | | |
| | CFA15_2 | 0.167 ± 0.081 | 2.050 | 4.1 × 10⁻² | 0.002 | | |
| | CFA15_2 × CFA20 | 0.161 ± 0.049 | 3.291 | 1.0 × 10⁻³ | 0.005 | | |
| | CFA15_2 × CFA21_red_dom | −0.139 ± 0.079 | −1.752 | 8.0 × 10⁻² | 0.001 | | |
| | CFA18_red_dom | 1.225 ± 0.217 | 5.65 | <2.2 × 10⁻¹⁶ | 0.015 | | |
| | CFA18_red_dom:CFA20 | −0.381 ± 0.112 | −3.400 | 1.0 × 10⁻³ | 0.005 | | |
| | CFA18_red_dom:CFA21_red_dom | −0.308 ± 0.174 | −1.772 | 7.7 × 10⁻² | 0.001 | | |
| | CFA20 | 0.985 ± 0.034 | 28.85 | <2.2 × 10⁻¹⁶ | 0.281 | | |
| | CFA21_red_dom | 0.436 ± 0.055 | 7.944 | <2.2 × 10⁻¹⁶ | 0.029 | | |
| E. | Intercept | 1.134 ± 0.051 | 22.195 | <2.2 × 10₋₁₆ | — | 0.7346 | −2,775.53 |
| | CFA2 | 0.908 ± 0.026 | 35.043 | <2.2 × 10⁻¹⁶ | 0.365 | | |
| | CFA15_2 | 0.102 ± 0.073 | 1.387 | 1.67 × 10⁻¹ | 0.001 | | |
| | CFA15_2 × CFA20 | 0.148 ± 0.048 | 3.061 | 2.0 × 10⁻³ | 0.004 | | |
| | CFA18_red_dom | 1.017 ± 0.185 | 5.496 | <2.2 × 10⁻¹⁶ | 0.014 | | |
| | CFA18_red_dom × CFA20 | −0.406 ± 0.112 | −3.640 | <2.2 × 10⁻¹⁶ | 0.006 | | |
| | CFA20 | 0.992 ± 0.034 | 29.141 | <2.2 × 10⁻¹⁶ | 0.285 | | |

Table 19: Coefficients, coefficient standard error, t score values, t test p-values, and PRE for the y-intercept and each of the independent variables in the best fit linear model incorporating non-additivity and pairwise epistasis. Section A. shows the base model that assumes perfect additivity at each locus and no interactions between loci. Section B. shows the best fit model incorporating dominance at all five loci. Section C. shows a model consisting of only the two previously reported loci (CFA15 and CFA20) using their best dominance encoding, and their pairwise interaction (CFA15_2×CFA20). Section D. shows the best fit model incorporating both the dominance terms in model B. and two pairwise epistasis terms: CFA15_2×CFA20 and CFA18_red_dom×CFA20. Section E. shows a reduced version of model D. that only includes terms that explained >0.1% of variance (PRE>1×10⁻³) in model D. and shows similar performance.

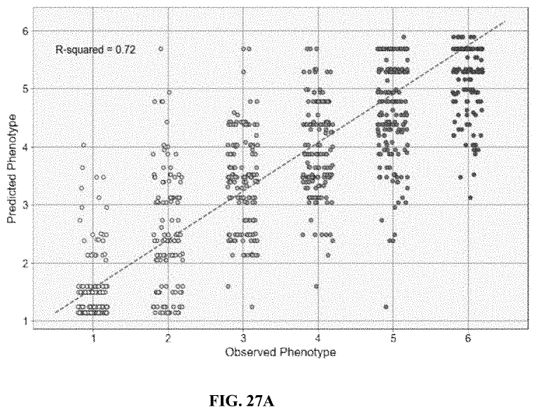

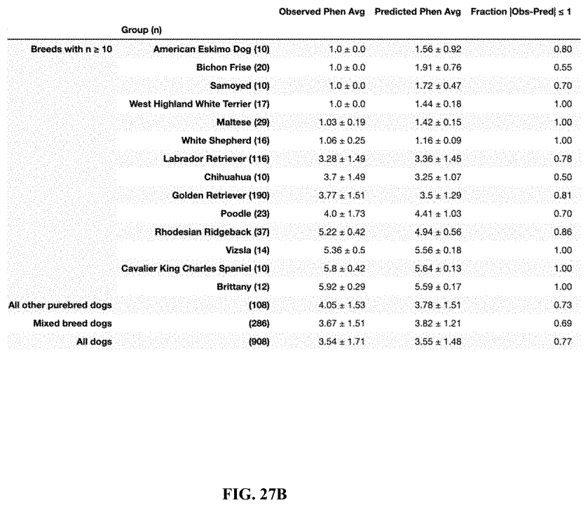

Next, 4,095 machine learning classifier models were trained with each possible combination of the seven statistically significant pairwise epistatic interactions and the five loci in the best fit dominance model. A model using the best dominance encodings for only the two previously reported loci—CFA15_2 and CFA20—and their pairwise interaction explained 54% of variance (Adjusted R-squared=0.5394) (Table 19, Section C). The model with the highest adjusted R-squared value (0.7353) included terms for each of the five loci in the best fit dominance model as well as interaction terms for CFA15_2×CFA20, CFA15_2×CFA21, CFA18_red_dom×CFA20, and CFA18_red_dom×CFA21_red_dom (Table 19, Section D). However, three terms accounted for less than 1% the total variance each: CFA15_2, CFA15_2×CFA21, and CFA18_red_dom×CFA21_red_dom. A reduced model excluding these terms (Table 19, Section E) was not significantly less predictive than the full best fit model (Table 19, Section D) (likelihood ratio test p-value=7.70×10⁻²), and was significantly more predictive than either the purely additive model (Table 19, Section A) (likelihood ratio test p-value=2.595×10⁻⁹) or the model with the best fit dominance encoding and no epistasis (Table 19, Section B) (likelihood ratio test p-value=5.104×10⁻⁶). The reduced best fit predictive model was applied to the 908 dogs in the validation sample, and it was observed to be able to explain 72% (adjusted R-squared=0.7211) of variation in coat pheomelanin intensity across all dogs (FIG. 27A).

FIGS. 27A-27B show performance of the best fit multivariate linear regression classifier model for pheomelanin intensity phenotypes in validation cohort. FIG. 27A: Strip plot of observed versus predicted phenotypes for all dogs in the validation dataset using the predictive model shown in Table 17. The adjusted R-squared value is shown in the top right hand corner. Each point represents a single dog, colored according to its observed six point phenotype. FIG. 27B: Performance of the multivariate linear regression model within and across breeds. For each row, observed and predicted phenotype averages are shown±their standard deviation. To assess model prediction accuracy in each breed or group, each row shows the fraction of dogs with a predicted phenotype value within one point of their observed phenotype (on the six point phenotype scale).

In order to evaluate the classifier model's performance in specific breeds, some of which had insufficient sample sizes or phenotypic variation to calculate a meaningful R-squared value, the percentage of dogs in a breed for which the model predicted a phenotype value within 1 point of the observed phenotype value (FIG. 27B) was also calculated. This value was 77% across all validation dogs, and 69% across mixed breed validation dogs. Among purebred validation dogs, the model's performance was generally high in breeds that are fixed for a narrow range of coat pheomelanin intensity (e.g., Samoyeds and Irish Setters) and lower in breeds with a wide range of coat colors (e.g., Chihuahuas and Poodles). Some notable exceptions to this pattern were Bichon Frisé, which are fixed for cream or white coats but poorly predicted by this model, and Golden Retrievers and Yellow Labrador retrievers, which display nearly the full range of coat pheomelanin intensity variation and for which the model is highly predictive.

An understanding of the genetic basis of variable pheomelanin intensity in dog coat color has progressed recently with the discovery of associations between this phenotype and three genes: MC1R, MFSD12, and KITLG [refs. 17-18 and 31]. However, the entire genetic architecture of this apparently multigenic phenotype may remain obscure because the explanatory power of known variants in or near these genes may be mostly limited to a small number of breeds. The results described herein demonstrate that the hypothetical “I locus” controlling coat pheomelanin intensity variation actually maps to at least five separate genetic loci that together explain the majority of phenotypic variation in purebred and mixed breed dogs, including several breeds with highly variable coat pheomelanin intensity.

The top CFA2 variant falls within a long intergenic non-coding RNA (lincRNA) with unknown functional significance in domestic dog. Many mammalian (including dog) lincRNAs are known to modulate the expression of nearby protein-coding genes via cis-regulatory mechanisms [refs. 49-52]. The closest annotated canine protein-coding gene is RUNX family transcription factor 3 (RUNX3), located approximately 82 kb downstream of ENSCAFG00000042716 at CFA2: 74,829,960-74,856,947. RUNX3 encodes a transcription factor that shows reduced expression in hair follicles in human premature hair greying, and appears to regulate expression of several other genes that also show reduced expression in premature greying samples [ref 53]. RUNX3 is also known to be a regulator of hair shape determination during murine embryonic development [ref 54]. Therefore, the CFA2 locus identified in the GWAS may be tagging a cis-regulatory module comprising ENSCAFG00000042716, RUNX3, and possibly other unknown genic variants or functional genomic elements. Identifying the causal mutations underlying this association may be performed by fine mapping of the locus, as well as molecular experiments to directly assess the functional impacts of any candidate mutations.

The top CFA21 variant is an intronic substitution in the TYR gene. This gene encodes the enzyme tyrosinase, which catalyzes the oxidation of 1-dihydroxy-phenylalanine (DOPA) to DOPA quinone, a precursor of both eumelanin and pheomelanin. Mutations in and around TYR produce varying degrees of pheomelanin dilution in several mammalian species by decreasing the amount of pheomelanin produced in hair shaft melanosomes [refs. 21-30]. Canine geneticists have previously hypothesized that TYR mutations might also produce pheomelanin dilution in dogs [ref 55], but earlier candidate-gene studies of exonic variants in the gene did not uncover any associated variants [ref 20]. However, the hypothesis that TYR variants can modulate coat pheomelanin intensity in dogs was supported by an identification of a missense mutation in the TYR gene as causal for a unique temperature-dependent pigment dilution phenotype (acromelanism) in a single dog [ref 56]. The results described herein solidifies this hypothesis and provides the first documented link between canine TYR variants and non-temperature-dependent coat pheomelanin intensity variation. Fine mapping and functional validation may be performed to definitively identify a causal variant. In multiple species, some of the genes located nearby TYR on CFA21 (including NOX4 [ref 57] and GRA45 [refs. 58-59]) are also known to be involved in skin pigmentation, so it is also possible that other variants outside of the TYR gene may be driving or contributing to the association signal on CFA21.

The connection between coat pheomelanin intensity and the gene tagged by the top CFA18 association is less apparent. The A to G substitution at this variant results in an amino acid substitution from isoleucine to methionine in the solute carrier family 26 member 4 (SLC26A4) protein. Based on computational modeling (Sorting Intolerant from Tolerant (SIFT) score=0.03), this substitution is predicted to be somewhat deleterious [ref 60]. However, its functional consequences in dogs have not been reported. While the SLC26A4 gene has no clear connection to hair coat pigmentation in mammals, it does play a role in a variety of hearing impairment phenotypes in human and inner ear abnormalities in mouse, including hyperpigmentation in the stria vascularis [ref 61] and degeneration of inner ear hair cells [ref 62]. There is substantial precedent for genes that affect inner ear function also affecting canine coat color: certain mutations in and around the microphthalmia-associated transcription factor (MITE) [ref 13] and PMEL (also known as SILV) [ref 10] genes, which are responsible for the piebald and merle coat patterns (respectively), cause varying degrees of deafness due to insufficient pigment expression in specialized hairs in the inner ear [refs. 10 and 63]. Additionally, mutations in and around KITLG cause hearing loss in humans [ref 64]. Due to its low minor allele frequency in the dataset (5%), the top CFA18 GWAS marker only explains 4% of variance in the intensity phenotype across all dogs, but still has a significant effect size both the GWAS and the predictive model. It is most variable in purebred Poodles, where it has a minor allele frequency of 46% (FIG. 25B). This association will require additional validation, ideally in a larger panel of purebred Poodles.

Significant evidence was found for epistatic interactions between the CFA20 locus and both the CFA15 and CFA18 loci. In fact, based on the PRE values in the linear regression analysis, the effect of the CFA15×CFA20 interaction is greater than the effect of the top CFA15 variant (Table 19, Sections C-E). Based on what is currently known about the molecular functions of the three genes closest to these variants, it is unclear exactly how these epistatic relationships might arise: The KITLG gene on CFA15 encodes a ligand that binds to the c-Kit protein on the surface of melanocytes, triggering the Ras/MAPK signalling pathway and stimulating melanocyte proliferation and melanogenesis [refs. 65-67]. The CFA15 CNV that the GWAS signal appears to be tagging falls upstream of the dog KITLG coding sequence, indicating that its likely affecting pheomelanin intensity by modulating KITLG expression. As noted in [ref 31], this assertion is supported by the fact that genetic variants that alter the expression of KITLG have been associated with both pheomelanin and eumelanin dilution in several mammalian species [refs. 66 and 68-72]. The SLC26A4 gene on CFA18 encodes a transmembrane ion transporter that is highly expressed on the apical surfaces of epithelial cells in the inner ear [ref 73], thyroid [ref 74], and kidney [ref 75] in humans and mice. As mentioned above, mutations in SLC26A4 have been associated with abnormal melanin deposition and hair cell degeneration in the inner ear. Unfortunately, little is known about the role that SLC26A4 plays in these phenotypes. It is also possible that the GWAS signal on CFA18 is actually driven by some other nearby gene that happens to be in high linkage disequilibrium with the top CFA18 variant in this study sample. The MFSD12 gene on CFA20 encodes a transmembrane solute transporter that localizes to melanocyte lysosomes and/or late endosomes in mice [ref 76]. The molecular mechanism by which MFSD12 influences hair pigmentation is still not well understood, but it may regulate melanosome autophagy [ref 76]. If this is the case, then it is possible that the MFSD12 cream-associated variant masks the effect of the KITLG red-associated variant by causing abnormal degradation of melanosomes downstream of pro-melanogenic signalling by KITLG.

A multigenic predictive model using genotypes at the most strongly-associated single-nucleotide genetic markers on CFA2, 15, 18, 20, and 21, plus two interaction terms was able to explain over 70% of the phenotypic variation across both the GWAS cohort and an independent validation cohort containing individuals from over 60 breeds as well as mixed breed dogs. This represents a gain of approximately 20% variance explained compared to a model using only the two previously discovered loci (Table 19, Section C). Because coat pheomelanin intensity appears to be a truly continuous phenotype across dogs, it is likely that the remaining variation is controlled by multiple additional loci. Currently, the only other known canine pheomelanin intensity loci are two highly breed-specific mutations in the MC1R gene, which underlie cream coats in Siberian Huskies and Australian Cattle Dogs [ref 17]. These variants were not typed on the genotyping array, so they were not included in these analyses. The study did not incorporate the progressive “fading” phenotype seen in several dog breeds—most notably Poodles—in which coat pigmentation lightens as a dog reaches adulthood. It is unclear if and to what extent this hypothetical dominant trait affects or interacts with pheomelanin intensity. The fading phenotypes of dogs in the study are unknown, but connections may be revealed between progressive fading and coat pheomelanin intensity variation.

Taken together, these results demonstrate that coat pheomelanin intensity in the domestic dog is a multigenic trait both across and within breeds, and that some loci controlling this trait likely interact via unknown biological pathways. Further fine mapping and experimental investigation may be performed to validate the three novel associations, to characterize the roles these and other genetic loci play in pigmentation in dogs and other species, and to determine whether any mutations associated with coat pheomelanin intensity variation also exhibit pleiotropic effects on canine health, such as deafness.

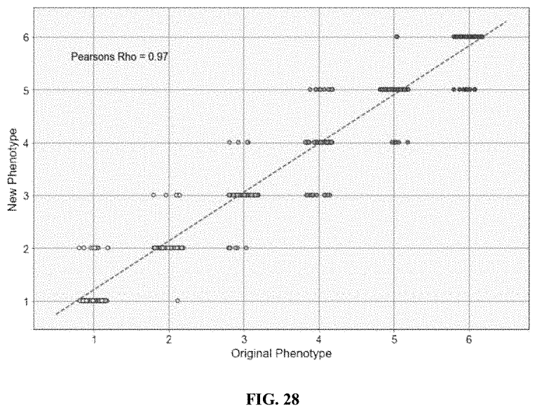

FIG. 28 shows phenotyping validation on 350 randomly selected dogs. A strip plot shows original versus re-scored 6 point phenotypes for a random sample of 350 dogs from the discovery sample. The correlation coefficient (Pearson's Rho) between the original and new phenotype scores is shown in the upper left hand corner of the plot.

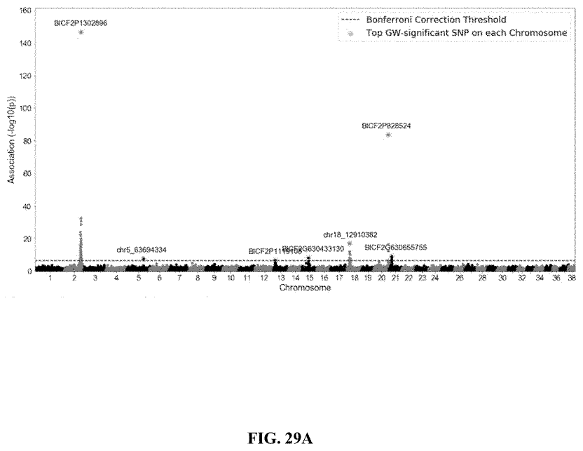

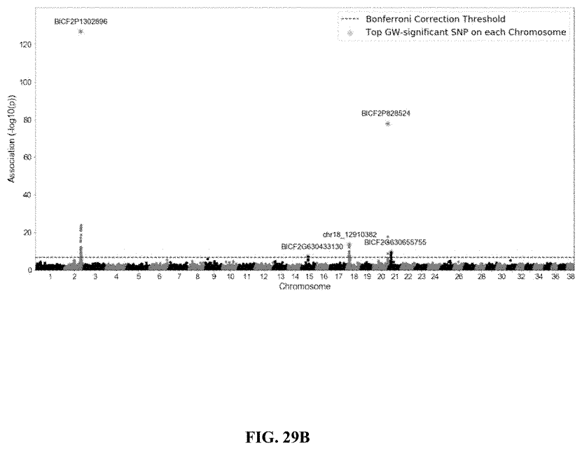

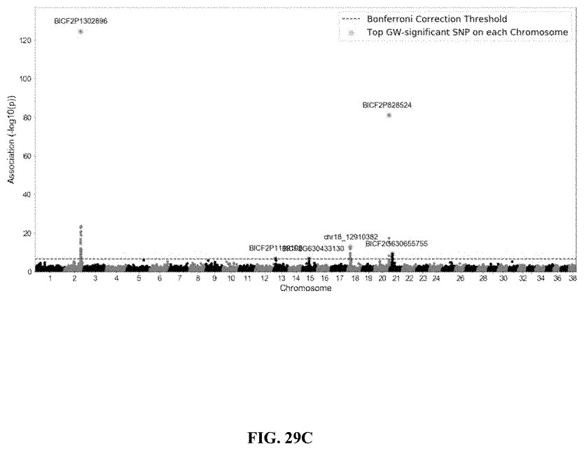

FIGS. 29A-29C show Manhattan plots for additional GWAS, including 6-point phenotype, no covariates (FIG. 29A); binary phenotype, with covariates (FIG. 29B); and binary phenotype, no covariates (FIG. 29C).

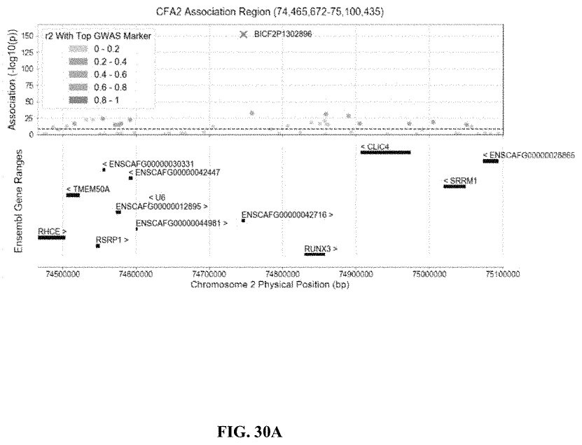

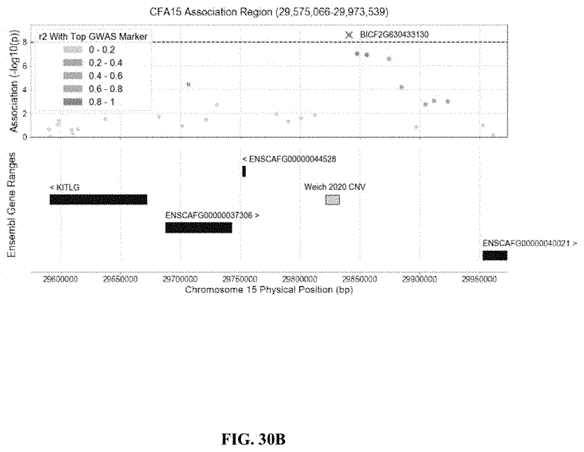

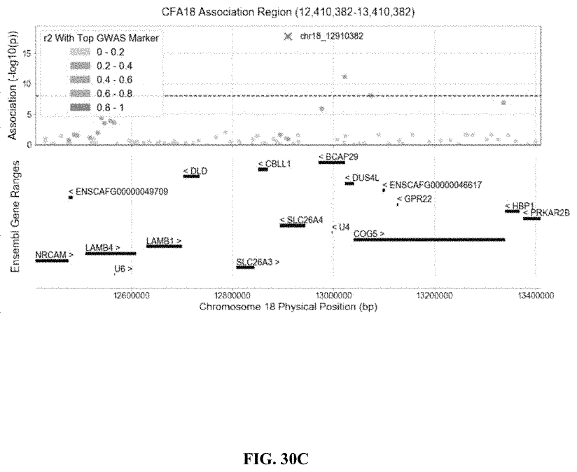

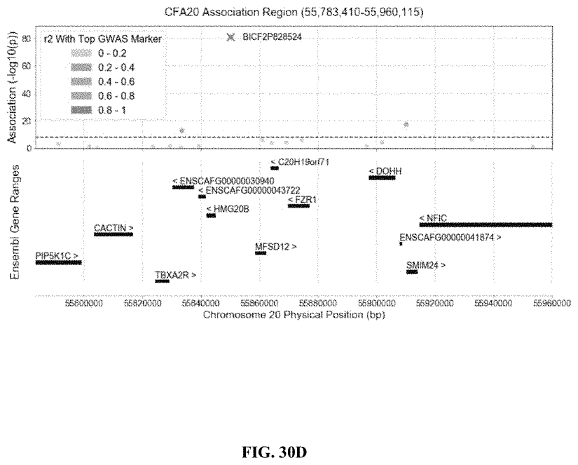

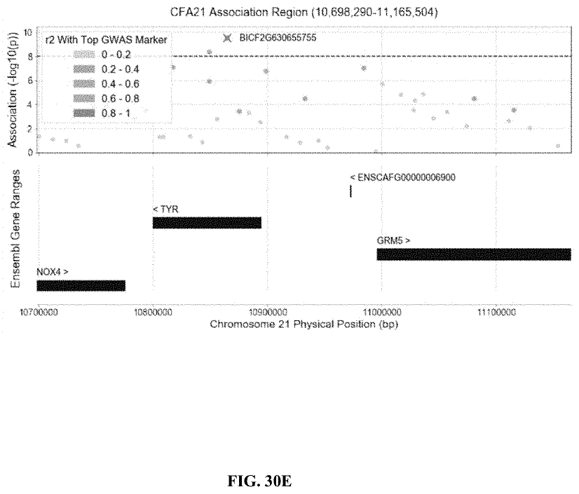

FIGS. 30A-30E show detailed views of regions surrounding top GWAS SNPs (e.g., on CFA2, CFA15, CFA18, CFA20, and CFA21), including CFA2 Association Region (74,465,672-75,100,435) (FIG. 30A); CFA15 Association Region (29,575,066-29,973,539) (FIG. 30B); CFA18 Association Region (12,410,382-13,410,382) (FIG. 30C); CFA20 Association Region (55,783,410-55,960,115) (FIG. 30D); and CFA21 Association Region (10,698,290-11,165,504) (FIG. 30E). Each panel shows the genomic region defined by the positions of the first upstream marker and last downstream marker with r2≥0.2 with the most significant GWAS marker on the chromosome (indicated by a red “x”). The top panel of each figure shows the GWAS −log₁₀(p-value) and physical position of all GWAS markers in the region, colored by their r² with the top GWAS marker. The bottom panel of each figure shows the canFam3.1 locations of known dog transcripts in this region. Transcription ranges are shown as dark blue rectangles, each of which is labelled with its Ensembl transcript or gene name and its strand orientation (“>”=plus strand, “<”=minus strand).

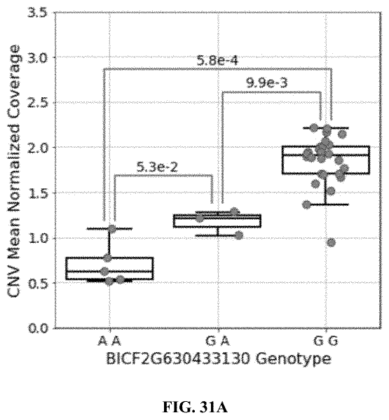

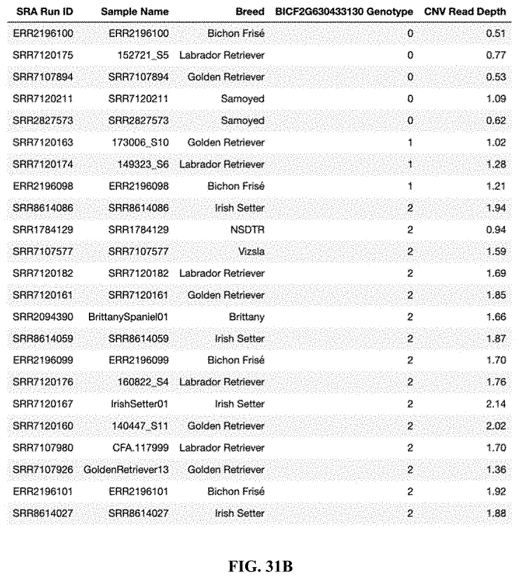

FIG. 31A shows that CFA15 top marker genotype correlates with sequencing coverage in known CNV. Boxplots overlaid with strip plots show the distribution of mean normalized depth of coverage across the CFA15 CNV characterized in [ref 31] (CFA15: 29,821,450-29,832,950 bp) for dogs with each possible BICF2G630433130 genotype. Each point represents a single dog. Kruskal Wallis test p-values are shown for each pair of genotypes.

FIG. 31B shows SRA run ID and sample name, breed, BICF2G630433130 genotype (coded as number of red-associated alleles), and CFA15 CNV mean normalized depth of coverage for all dogs shown in FIG. 31A.

### REFERENCES

[Ref 1] Wang G, Zhai W, Yang H, Fan R, Cao X, Zhong L, et al. The genomics of selection in dogs and the parallel evolution between dogs and humans. Nat Commun. 2013 Jun.; 4(1):1860 is incorporated by reference herein in its entirety.
[Ref 2] MacLean E L, Snyder-Mackler N, vonHoldt B M, Serpell J A. Highly heritable and functionally relevant breed differences in dog behaviour. Proc Biol Sci. 2019 09; 286(1912):20190716 is incorporated by reference herein in its entirety.
[Ref 3] Columella LIM, Forster E S. On agriculture: in three volumes. 2: Res rustica V-IX. Reprinted. Cambridge, Mass.: Harvard Univ. Press; 2010. 503 p. (The Loeb Classical Library) is incorporated by reference herein in its entirety.
[Ref 4] United Kennel Club. CHESAPEAKE BAY RETRIEVER Official UKC Breed Standard [Internet]. United Kennel Club; [cited 2020 Sep. 16]. Available from: www.ukcdogs.com/docs/breeds/chesapeake-bay-retriever.pdf is incorporated by reference herein in its entirety.
[Ref 5] Newton J M, Wilkie A L, He L, Jordan S A, Metallinos D L, Holmes N G, et al. Melanocortin 1 receptor variation in the domestic dog. Mamm Genome. 2000 Jan.; 11(1):24-30 is incorporated by reference herein in its entirety.
[Ref 6] Schmutz S M, Berryere T G, Goldfinch A D. TYRP1 and MC1R genotypes and their effects on coat color in dogs. Mamm Genome. 2002 July; 13(7):380-7 is incorporated by reference herein in its entirety.
[Ref 7] Schmutz S M, Berryere T G, Ellinwood N M, Kerns J A, Barsh G S. MC1R studies in dogs with melanistic mask or brindle patterns. J Hered. 2003 February; 94(1):69-73 is incorporated by reference herein in its entirety.
[Ref 8] Berryere T G, Kerns J A, Barsh G S, Schmutz S M. Association of an Agouti allele with fawn or sable coat color in domestic dogs. Mamm Genome. 2005 April; 16(4):262-72 is incorporated by reference herein in its entirety.
[Ref 9] Kerns J A, Newton J, Berryere T G, Rubin E M, Cheng J-F, Schmutz S M, et al. Characterization of the dog Agouti gene and a nonagoutimutation in German Shepherd Dogs. Mamm Genome. 2004 Oct.; 15(10):798-808 is incorporated by reference herein in its entirety.
[Ref 10] Clark L A, Wahl J M, Rees C A, Murphy K E. From The Cover: Retrotransposon insertion in SILV is responsible for merle patterning of the domestic dog. Proceedings of the National Academy of Sciences. 2006 Jan. 31; 103(5):1376-81 is incorporated by reference herein in its entirety.
[Ref 11] Candille S I, Kaelin C B, Cattanach B M, Yu B, Thompson D A, Nix M A, et al. A -Defensin Mutation Causes Black Coat Color in Domestic Dogs. Science. 2007 Nov. 30; 318(5855):1418-23 is incorporated by reference herein in its entirety.
[Ref 12] Drögemüller C, Philipp U, Haase B, Günzel-Apel A-R, Leeb T. A noncoding melanophilin gene (MLPH) SNP at the splice donor of exon 1 represents a candidate causal mutation for coat color dilution in dogs. J Hered. 2007; 98(5):468-73 is incorporated by reference herein in its entirety.
[Ref 13] Karlsson E K, Baranowska I, Wade C M, Salmon Hillbertz NHC, Zody M C, Anderson N, et al. Efficient mapping of mendelian traits in dogs through genome-wide association. Nat Genet. 2007 November; 39(11):1321-8 is incorporated by reference herein in its entirety.
[Ref 14] Kerns J A, Cargill E J, Clark L A, Candille S I, Berryere T G, Olivier M, et al. Linkage and segregation analysis of black and brindle coat color in domestic dogs. Genetics. 2007 July; 176(3):1679-89 is incorporated by reference herein in its entirety.
[Ref 15] Dreger D L, Schmutz S M. A SINE insertion causes the black-and-tan and saddle tan phenotypes in domestic dogs. J Hered. 2011 October; 102 Suppl 1:S11-18 is incorporated by reference herein in its entirety.
[Ref 16] Baranowska Körberg I, Sundström E, Meadows J R S, Rosengren Pielberg G, Gustafson U, Hedhammar A, et al. A Simple Repeat Polymorphism in the MITF-M Promoter Is a Key Regulator of White Spotting in Dogs. Murphy W J, editor. PLoS ONE. 2014 Aug. 12; 9(8):e104363 is incorporated by reference herein in its entirety.
[Ref 17] Dung N, Letko A, Lepori V, Hadji Rasouliha S, Loechel R, Kehl A, et al. Two MC1R loss-of-function alleles in cream-coloured Australian Cattle Dogs and white Huskies. Anim Genet. 2018 August; 49(4):284-90 is incorporated by reference herein in its entirety.
[Ref 18] Hédan B, Cadieu E, Botherel N, Dufaure de Citres C, Letko A, Rimbault M, et al. Identification of a Missense Variant in MFSD12 Involved in Dilution of Phaeomelanin Leading to White or Cream Coat Color in Dogs. Genes (Basel). 2019 21; 10(5) is incorporated by reference herein in its entirety.
[Ref 19] Sponenberg D P, Rothschild M F. Genetics of coat colour and hair texture. In: Ruvinsky A, Sampson J, editors. The genetics of the dog. Wallingford: CABI; 2001. p. 61-85 is incorporated by reference herein in its entirety.
[Ref 20] Schmutz S M, Berryere T G. The genetics of cream coat color in dogs. J Hered. 2007; 98(5):544-8 is incorporated by reference herein in its entirety.
[Ref 21] Kwon B S, Halaban R, Chintamaneni C. Molecular basis of mouse Himalayan mutation. Biochem Biophys Res Commun. 1989 May 30; 161(1):252-60 is incorporated by reference herein in its entirety.
[Ref 22] Yokoyama T, Silversides D W, Waymire K G, Kwon B S, Takeuchi T, Overbeek P A. Conserved cysteine to serine mutation in tyrosinase is responsible for the classical albino mutation in laboratory mice. Nucleic Acids Res. 1990 Dec. 25; 18(24):7293-8 is incorporated by reference herein in its entirety.
[Ref 23] Fukai K, Holmes S A, Lucchese N J, Siu V M, Weleber R G, Schnur R E, et al. Autosomal recessive ocular albinism associated with a functionally significant tyrosinase gene polymorphism. Nat Genet. 1995 Jan.; 9(1):92-5 is incorporated by reference herein in its entirety.
[Ref 24] Aigner B, Besenfelder U, Müller M, Brem G. Tyrosinase gene variants in different rabbit strains. Mamm Genome. 2000 August; 11(8):700-2 is incorporated by reference herein in its entirety.
[Ref 25] Schmutz S M, Berryere T G, Ciobanu D C, Mileham A J, Schmidtz B H, Fredholm M. A form of albinism in cattle is caused by a tyrosinase frameshift mutation. Mamm Genome. 2004 Jan.; 15(1):62-7.1 is incorporated by reference herein in its entirety.
[Ref 26] Lyons L A, Imes D L, Rah H C, Grahn R A. Tyrosinase mutations associated with Siamese and Burmese patterns in the domestic cat (Felis catus). Animal Genetics. 2005 April; 36(2):119-26 is incorporated by reference herein in its entirety.
[Ref 27] Schmidt-Küntzel A, Eizirik E, O'Brien S J, Menotti-Raymond M. Tyrosinase and Tyrosinase Related Protein 1 Alleles Specify Domestic Cat Coat Color Phenotypes of the albino and brown Loci. Journal of Heredity. 2005 Jun. 1; 96(4):289-301 is incorporated by reference herein in its entirety.
[Ref 28] Imes D L, Geary L A, Grahn R A, Lyons L A. Albinism in the domestic cat (Felis catus) is associated with a tyrosinase (TYR) mutation. Anim Genet. 2006 April; 37(2):175-8 is incorporated by reference herein in its entirety.
[Ref 29] Anello M, Fernández E, Daverio M S, Vidal-Rioja L, Di Rocco F. TYR Gene in Llamas: Polymorphisms and Expression Study in Different Color Phenotypes. Front Genet. 2019; 10:568 is incorporated by reference herein in its entirety.
[Ref 30] Yu Y, Grahn R A, Lyons L A. Mocha tyrosinase variant: a new flavour of cat coat coloration. Anim Genet. 2019 April; 50(2):182-6 is incorporated by reference herein in its entirety.
[Ref 31] Weich K, Affolter V, York D, Rebhun R, Grahn R, Kallenberg A, et al. Pigment Intensity in Dogs is Associated with a Copy Number Variant Upstream of KITLG. Genes. 2020 Jan. 9; 11(1):75 is incorporated by reference herein in its entirety.
[Ref 32] AKC Staff. The Most Popular Dog Breeds of 2019 [Internet]. [cited 2020 Sep. 16]. Available from: www.akc.org/expert-advice/dog-breeds/2020-popular-breeds-2019/is incorporated by reference herein in its entirety.
[Ref 33] Hunter J D. Matplotlib: A 2D Graphics Environment. Comput Sci Eng. 2007; 9(3):90-5 is incorporated by reference herein in its entirety.
[Ref 34] Reback J, McKinney W, Jbrockmendel, Bossche J V D, Augspurger T, Cloud P, et al. pandas-dev/pandas: Pandas 1.2.3 [Internet]. Zenodo; 2021 [cited 2021 Mar. 9]. Available from: zenodo.org/record/3509134 is incorporated by reference herein in its entirety.
[Ref 35] Chang C C, Chow C C, Tellier L C, Vattikuti S, Purcell S M, Lee J J. Second-generation PLINK: rising to the challenge of larger and richer datasets. Gigascience. 2015; 4:7 is incorporated by reference herein in its entirety.
[Ref 36] Zhou X, Stephens M. Genome-wide efficient mixed-model analysis for association studies. Nat Genet. 2012 Jun. 17; 44(7):821-4 is incorporated by reference herein in its entirety.
[Ref 37] Cadieu E, Neff M W, Quignon P, Walsh K, Chase K, Parker H G, et al. Coat variation in the domestic dog is governed by variants in three genes. Science. 2009 Oct. 2; 326(5949):150-3 is incorporated by reference herein in its entirety.
[Ref 38] National Center for Biotechnology Information. NCBI Sequence Read Archive [Internet]. Available from: www.ncbi.nlm.nih.gov/sra/ is incorporated by reference herein in its entirety.
[Ref 39] Li H, Durbin R. Fast and accurate long-read alignment with Burrows-Wheeler transform. Bioinformatics. 2010 Mar. 1; 26(5):589-95 is incorporated by reference herein in its entirety.
[Ref 40] Broad Institute. Picard Tools [Internet]. The Broad Institute; Available from: broadinstitute.github.io/picard/ is incorporated by reference herein in its entirety.
[Ref 41] McKenna A, Hanna M, Banks E, Sivachenko A, Cibulskis K, Kernytsky A, et al. The Genome Analysis Toolkit: A MapReduce framework for analyzing next-generation DNA sequencing data. Genome Research. 2010 Sep. 1; 20(9):1297-303 is incorporated by reference herein in its entirety.
[Ref 42] Fisher R A. XV.—The Correlation between Relatives on the Supposition of Mendelian Inheritance. Trans R Soc Edinb. 1919; 52(2):399-433 is incorporated by reference herein in its entirety.
[Ref 43] Pedregosa F, Varoquaux G, Gramfort A, Michel V, Thirion B, Grisel O, et al. Scikit-learn: Machine Learning in Python. Journal of Machine Learning Research. 2011; 12(85):2825-30 is incorporated by reference herein in its entirety.
[Ref 44] Karyadi D M, Karlins E, Decker B, vonHoldt B M, Carpintero-Ramirez G, Parker H G, et al. A copy number variant at the KITLG locus likely confers risk for canine squamous cell carcinoma of the digit. PLoS Genet. 2013 Mar.; 9(3):e1003409 is incorporated by reference herein in its entirety.
[Ref 45] Meuwissen T H, Hayes B J, Goddard M E. Prediction of total genetic value using genome-wide dense marker maps. Genetics. 2001 April; 157(4):1819-29 is incorporated by reference herein in its entirety.
[Ref 46] de los Campos G, Hickey J M, Pong-Wong R, Daetwyler H D, Calus MPL. Whole-Genome Regression and Prediction Methods Applied to Plant and Animal Breeding. Genetics. 2013 February; 193(2):327-45 is incorporated by reference herein in its entirety.
[Ref 47] Hayward J J, White M E, Boyle M, Shannon L M, Casal M L, Castelhano M G, et al. Imputation of canine genotype array data using 365 whole-genome sequences improves power of genome-wide association studies. Barsh G S, editor. PLoS Genet. 2019 Sep. 16; 15(9):e1008003 is incorporated by reference herein in its entirety.
[Ref 48] Weller J I, Glick G, Shirak A, Ezra E, Seroussi E, Shemesh M, et al. Predictive ability of selected subsets of single nucleotide polymorphisms (SNPs) in a moderately sized dairy cattle population. Animal. 2014 Feb.; 8(2):208-16 is incorporated by reference herein in its entirety.
[Ref 49] Engreitz J M, Haines J E, Perez E M, Munson G, Chen J, Kane M, et al. Local regulation of gene expression by lncRNA promoters, transcription and splicing. Nature. 2016 17; 539(7629):452-5 is incorporated by reference herein in its entirety.
[Ref 50] Li Y, Shan Z, Yang B, Yang D, Men C, Cui Y, et al. LncRNA HULC promotes epithelial and smooth-muscle-like differentiation of adipose-derived stem cells by upregulation of BMP9. Pharmazie. 2018 Jan. 2; 73(1):49-55 is incorporated by reference herein in its entirety.
[Ref 51] Hitte C, Le Béguec C, Cadieu E, Wucher V, Primot A, Prouteau A, et al. Genome-Wide Analysis of Long Non-Coding RNA Profiles in Canine Oral Melanomas. Genes (Basel). 2019 23; 10(6) is incorporated by reference herein in its entirety.
[Ref 52] Whitaker D T, Ostrander E A. Hair of the Dog: Identification of a Cis-Regulatory Module Predicted to Influence Canine Coat Composition. Genes (Basel). 2019 26; 10(5) is incorporated by reference herein in its entirety.
[Ref 53] Bian Y, Wei G, Song X, Yuan L, Chen H, Ni T, et al. Global downregulation of pigmentation-associated genes in human premature hair graying. Exp Ther Med. 2019 August; 18(2):1155-63 is incorporated by reference herein in its entirety.
[Ref 54] Raveh E, Cohen S, Levanon D, Groner Y, Gat U. Runx3 is involved in hair shape determination. Dev Dyn. 2005 August; 233(4):1478-87 is incorporated by reference herein in its entirety.
[Ref 55] Little C C. The Inheritance of Coat Color in Dogs. Comstock Pub. Associates; 1957 is incorporated by reference herein in its entirety.
[Ref 56] Bychkova E, Viktorovskaya O, Filippova E, Eliseeva Z, Barabanova L, Sotskaya M, et al. Identification of a candidate genetic variant for the Himalayan color pattern in dogs. Gene. 2020 October; 145212 is incorporated by reference herein in its entirety.
[Ref 57] Liu G-S, Peshavariya H, Higuchi M, Brewer A C, Chang C W T, Chan E C, et al. Microphthalmia-associated transcription factor modulates expression of NADPH oxidase type 4: a negative regulator of melanogenesis. Free Radic Biol Med. 2012 May 1; 52(9):1835-43 is incorporated by reference herein in its entirety.
[Ref 58] Nan H, Kraft P, Qureshi A A, Guo Q, Chen C, Hankinson S E, et al. Genome-Wide Association Study of Tanning Phenotype in a Population of European Ancestry. Journal of Investigative Dermatology. 2009 September; 129(9):2250-7 is incorporated by reference herein in its entirety.
[Ref 59] Adhikari K, Mendoza-Revilla J, Sohail A, Fuentes-Guajardo M, Lampert J, Chacon-Duque J C, et al. A GWAS in Latin Americans highlights the convergent evolution of lighter skin pigmentation in Eurasia. Nat Commun. 2019 21; 10(1):358 is incorporated by reference herein in its entirety.
[Ref 60] Ng P C, Henikoff S. SIFT: Predicting amino acid changes that affect protein function. Nucleic Acids Res. 2003 Jul. 1; 31(13):3812-4 is incorporated by reference herein in its entirety.
[Ref 61] Everett L A, Glaser B, Beck J C, Idol J R, Buchs A, Heyman M, et al. Pendred syndrome is caused by mutations in a putative sulphate transporter gene (PDS). Nat Genet. 1997 Dec.; 17(4):411-22 is incorporated by reference herein in its entirety.
[Ref 62] Lu Y-C, Wu C-C, Shen W-S, Yang T-H, Yeh T-H, Chen P-J, et al. Establishment of a knock-in mouse model with the SLC26A4 c.919-2A>G mutation and characterization of its pathology. PLoS One. 2011; 6(7):e22150 is incorporated by reference herein in its entirety.
[Ref 63] Stritzel S, Wohlke A, Distl O. A role of the microphthalmia-associated transcription factor in congenital sensorineural deafness and eye pigmentation in Dalmatian dogs. J Anim Breed Genet. 2009 February; 126(1):59-62 is incorporated by reference herein in its entirety.
[Ref 64] Zazo Seco C, Serraõ de Castro L, van Nierop J W, Morin M, Jhangiani S, Verver E J J, et al. Allelic Mutations of KITLG, Encoding KIT Ligand, Cause Asymmetric and Unilateral Hearing Loss and Waardenburg Syndrome Type 2. Am J Hum Genet. 2015 Nov. 5; 97(5):647-60 is incorporated by reference herein in its entirety.
[Ref 65] Grichnik J M, Burch J A, Burchette J, Shea C R. The SCF/KIT Pathway Plays a Critical Role in the Control of Normal Human Melanocyte Homeostasis. Journal of Investigative Dermatology. 1998 August; 111(2):233-8 is incorporated by reference herein in its entirety.
[Ref 66] Kunisada T, Yoshida H, Yamazaki H, Miyamoto A, Hemmi H, Nishimura E, et al. Transgene expression of steel factor in the basal layer of epidermis promotes survival, proliferation, differentiation and migration of melanocyte precursors. Development. 1998 August; 125(15):2915-23 is incorporated by reference herein in its entirety.
[Ref 67] Liao C-P, Booker R C, Morrison S J, Le L Q. Identification of hair shaft progenitors that create a niche for hair pigmentation. Genes Dev. 2017 Apr. 15; 31(8):744-56 is incorporated by reference herein in its entirety.
[Ref 68] Sarvella P A, Russell L B. STEEL, A NEW DOMINANT GENE I N THE HOUSE MOUSE. Journal of Heredity. 1956 May; 47(3):123-8 is incorporated by reference herein in its entirety.
[Ref 69] Bedell M A, Brannan C I, Evans E P, Copeland N G, Jenkins N A, Donovan P J. DNA rearrangements located over 100 kb 5′ of the Steel (S1)-coding region in Steel-panda and Steel-contrasted mice deregulate S1 expression and cause female sterility by disrupting ovarian follicle development. Genes Dev. 1995 Feb. 15; 9(4):455-70 is incorporated by reference herein in its entirety.
[Ref 70] Guenther C A, Tasic B, Luo L, Bedell M A, Kingsley D M. A molecular basis for classic blond hair color in Europeans. Nat Genet. 2014 July; 46(7):748-52 is incorporated by reference herein in its entirety.
[Ref 71] Song X, Xu C, Liu Z, Yue Z, Liu L, Yang T, et al. Comparative Transcriptome Analysis of Mink (Neovison vison) Skin Reveals the Key Genes Involved in the Melanogenesis of Black and White Coat Colour. Sci Rep. 2017 Dec.; 7(1):12461 is incorporated by reference herein in its entirety.
[Ref 72] Wu S, Li J, Ma T, Li J, Li Y, Jiang H, et al. MiR-27a regulates WNT3A and KITLG expression in Cashmere goats with different coat colors. Anim Biotechnol. 2019 Oct. 15; 1-8 is incorporated by reference herein in its entirety.
[Ref 73] Everett L A, Morsli H, Wu D K, Green E D. Expression pattern of the mouse ortholog of the Pendred's syndrome gene (Pds) suggests a key role for pendrin in the inner ear. Proc Natl Acad Sci USA. 1999 Aug. 17; 96(17):9727-32 is incorporated by reference herein in its entirety.
[Ref 74] Royaux I E, Suzuki K, Mori A, Katoh R, Everett L A, Kohn L D, et al. Pendrin, the protein encoded by the Pendred syndrome gene (PDS), is an apical porter of iodide in the thyroid and is regulated by thyroglobulin in FRTL-5 cells. Endocrinology. 2000 February; 141(2):839-45 is incorporated by reference herein in its entirety.
[Ref 75] Soleimani M, Greeley T, Petrovic S, Wang Z, Amlal H, Kopp P, et al. Pendrin: an apical Cl—/OH—/HCO3- exchanger in the kidney cortex. Am J Physiol Renal Physiol. 2001 February; 280(2):F356-364 is incorporated by reference herein in its entirety.
[Ref 76] Crawford N G, Kelly D E, Hansen M E B, Beltrame M H, Fan S, Bowman S L, et al. Loci associated with skin pigmentation identified in African populations. Science. 2017 17; 358(6365) is incorporated by reference herein in its entirety.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-51. (canceled)

52. A computer-implemented method for determining a pigmentation phenotype of a canine subject, comprising:
(a) receiving genotype data for the canine subject, wherein the genotype data comprises quantitative values of each of a plurality of genetic markers, wherein the plurality of genetic markers comprises genetic variants; and
(b) applying a trained machine learning algorithm to the genotype data to determine a predicted pigmentation phenotype based at least in part on the quantitative values of the plurality of genetic variants; and
(c) identifying the canine subject as having the predicted pigmentation phenotype.

53. The method of claim 52, wherein the canine subject is a dog.

54. The method of claim 53, wherein the dog is a purebred dog.

55. The method of claim 53, wherein the dog is a mixed breed dog.

56. The method of claim 52, further comprising obtaining the genotype data by assaying a biological sample obtained from the canine subject.

57. The method of claim 56, wherein the biological sample comprises a blood sample, a saliva sample, a swab sample, a cell sample, or a tissue sample.

58. The method of claim 56, wherein the assaying comprises sequencing the biological sample or derivatives thereof.

59. The method of claim 52, wherein the quantitative values are indicative of a presence or absence in the genotype data of each of the plurality of genetic variants.

60. The method of claim 52, wherein the plurality of genetic variants is selected from the group consisting of single nucleotide polymorphisms (SNPs), insertions or deletions (indels), microsatellites, and structural variants.

61. The method of claim 52, wherein the pigmentation phenotype comprises a coat color intensity phenotype, a ticking phenotype, a roaning phenotype, or a tongue pigmentation phenotype.

62. The method of claim 61, wherein the pigmentation phenotype comprises a coat color intensity phenotype.

63. The method of claim 62, wherein the plurality of genetic markers comprises one or more markers selected from the group listed in Table 8.

64. The method of claim 62, wherein the plurality of genetic markers comprises one or more SNPs of a genetic locus selected from the group consisting of canFam3.1 chr2: 74.7 Mb, chr20: 55.8 Mb, and chr21: 10.9 Mb.

65. The method of claim 61, wherein the pigmentation phenotype comprises a ticking phenotype.

66. The method of claim 61, wherein the pigmentation phenotype comprises a roaning phenotype.

67. The method of claim 66, wherein the plurality of genetic markers comprises one or more markers selected from the group listed in Table 11.

68. The method of claim 61, wherein the pigmentation phenotype comprises a tongue pigmentation phenotype.

69. The method of claim 68, wherein the plurality of genetic markers comprises one or more markers selected from the group listed in Table 13.

70. The method of claim 52, wherein applying the trained machine learning algorithm comprises determining a weighted sum of the quantitative values of the plurality of genetic markers.

71. The method of claim 70, wherein the weighted sum is determined using a plurality of pre-determined weights associated with the plurality of genetic markers.

72. The method of claim 71, wherein the plurality of pre-determined weights associated with the plurality of genetic markers is determined by performing a genome-wide association study (GWAS) comprising a multiple linear regression.

73. The method of claim 52, further comprising determining a second pigmentation phenotype of a second canine subject, and determining an expected range of pigmentation phenotypes of a potential offspring of the canine subject and the second canine subject.

74. The method of claim 73, further comprising determining a recommendation indicative of whether or not to breed the first canine subject and the second canine subject together, based on the expected range of pigmentation phenotypes of the potential offspring of the canine subject and the second canine subject.

75. The method of claim 74, further comprising determining a recommendation indicative of breeding the first canine subject and the second canine subject together, when the expected range of pigmentation phenotypes of the potential offspring of the canine subject and the second canine subject includes a pre-determined pigmentation phenotype.

76. The method of claim 74, further comprising determining a recommendation against breeding the first canine subject and the second canine subject together, when the expected range of pigmentation phenotypes of the potential offspring of the canine subject and the second canine subject does not include a pre-determined pigmentation phenotype.

77. The method of claim 74, further comprising generating a social connection between a first person associated with the first canine subject and a second person associated with the second canine subject, based at least in part on the expected range of pigmentation phenotypes of the potential offspring of the canine subject and the second canine subject.

78. The method of claim 52, further comprising identifying the canine subject as having the predicted pigmentation phenotype with an accuracy of at least about 70%.

79. The method of claim 52, wherein the trained machine learning algorithm comprises a linear regression or a logistic regression.

80. A computer system for determining a pigmentation phenotype of a canine subject, comprising:
a database that is configured to store genotype data for the canine subject, wherein the genotype data comprises quantitative values of each of a plurality of genetic markers, wherein the plurality of genetic markers comprises genetic variants; and
one or more computer processors operatively coupled to the database, wherein the one or more computer processors are individually or collectively programmed to:
(a) apply a trained machine learning algorithm to the genotype data to determine a predicted pigmentation phenotype based at least in part on the quantitative values of the plurality of genetic variants; and
(b) identify the canine subject as having the predicted pigmentation phenotype.

81. A non-transitory computer-readable medium comprising machine-executable code that, upon execution by one or more computer processors, implements a method for determining a coat color intensity phenotype of a canine subject, the method comprising:
(a) receiving genotype data for the canine subject, wherein the genotype data comprises quantitative values of each of a plurality of genetic markers, wherein the plurality of genetic markers comprises genetic variants;
(b) applying a trained machine learning algorithm to the genotype data to determine a predicted pigmentation phenotype based at least in part on the quantitative values of the plurality of genetic variants; and
(c) identifying the canine subject as having the predicted pigmentation phenotype.
